SEQUENCE LISTING (66 sequences)

SEQ ID NO: 1 - protein, 21 aa, Homo sapiens
NFELRDKSSRPGRNPKTGDVV

SEQ ID NO: 2 - protein, 21 aa, Homo sapiens
SLHHRQPRLGRNPKTGDSVNL

SEQ ID NO: 3 - protein, 60 aa, synthetic construct
  VARIANT 7: note = Any amino acid
  VARIANT 21: note = Any amino acid
  VARIANT 22: note = Any amino acid
  VARIANT 23: note = Any amino acid
  VARIANT 24: note = Any amino acid
  VARIANT 25: note = Any amino acid
  VARIANT 26: note = Any amino acid
  VARIANT 27: note = Any amino acid
  VARIANT 28: note = Any amino acid
  VARIANT 29: note = Any amino acid
  VARIANT 30: note = Any amino acid
  VARIANT 31: note = Any amino acid
  VARIANT 32: note = Any amino acid
  VARIANT 33: note = Any amino acid
  VARIANT 34: note = Any amino acid
  VARIANT 35: note = Any amino acid
  VARIANT 36: note = Any amino acid
  VARIANT 37: note = Any amino acid
  VARIANT 38: note = Any amino acid
  VARIANT 39: note = Any amino acid
  VARIANT 40: note = Any amino acid
  VARIANT 55: note = Any amino acid
  VARIANT 21..40: note = This region may encompass 1-20 residues
RPGRNPXTGDVVPVSARRVVXXXXXXXXXXXXXXXXXXXXFSLHHRQPRLGRNPXTGDSV

SEQ ID NO: 4 - protein, 60 aa, synthetic construct
  VARIANT 21: note = Any amino acid
  VARIANT 22: note = Any amino acid
  VARIANT 23: note = Any amino acid
  VARIANT 24: note = Any amino acid
  VARIANT 25: note = Any amino acid
  VARIANT 26: note = Any amino acid
  VARIANT 27: note = Any amino acid
  VARIANT 28: note = Any amino acid
  VARIANT 29: note = Any amino acid
  VARIANT 30: note = Any amino acid
  VARIANT 31: note = Any amino acid
  VARIANT 32: note = Any amino acid
  VARIANT 33: note = Any amino acid
  VARIANT 34: note = Any amino acid
  VARIANT 35: note = Any amino acid
  VARIANT 36: note = Any amino acid
  VARIANT 37: note = Any amino acid
  VARIANT 38: note = Any amino acid
  VARIANT 39: note = Any amino acid
  VARIANT 40: note = Any amino acid
  VARIANT 21..40: note = This region may encompass 1-20 residues
RPGRNPKTGDVVPVSARRVVXXXXXXXXXXXXXXXXXXXXFSLHHRQPRLGRNPKTGDSV

SEQ ID NO: 5 - protein, 44 aa, synthetic construct
RPGRNPKTGDVVPVSARRVVGPSLFSLHHRQPRLGRNPKTGDSV

SEQ ID NO: 6 - protein, 6 aa, synthetic construct
GGSGGS

SEQ ID NO: 7 - protein, 6 aa, synthetic construct
GPSLKL

SEQ ID NO: 8 - skipped

SEQ ID NO: 9 - protein, 473 aa, synthetic construct
MDPKGSLSWRILLFLSLAFELSYGEVKLVESGGGLVQPGGSLRLSCAASGFTFRTYAMSWVRQAPGKGLEWVATIGSDRRHTYYPDSVKGRFTISRDNAKNTLYLQMNSLRAEDTAVYYCVGPYDGYYGEFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 10 - protein, 473 aa, synthetic construct
MDPKGSLSWRILLFLSLAFELSYGEVQLVESGGGLVQPGGSLRLSCAASGFTFRTYAMSWVRQAPGKGLEWVATIGSDRRHTYYPDSVKGRFTISRDNSKNTLYLQMNSLRAEDTAVYYCVGPYDGYYGEFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 11 - protein, 473 aa, synthetic construct
MDPKGSLSWRILLFLSLAFELSYGEVKLVQSGAEVKKPGASVKVSCKASGFTFRTYAMSWVRQAPGQRLEWVATIGSDRRHTYYPDKFQGRVTITRDNAKNTLYMELSSLRSEDTAVYYCVGPYDGYYGEFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 12 - protein, 473 aa, synthetic construct
MDPKGSLSWRILLFLSLAFELSYGEVQLVESGGGLVKPGGSLRLSCAASGFTFSRYGMSWVRQAPGKGLEWVATISSGGSYTYYTDSVKGRFTISRDNAKNSLYLQMNSLRAEDTAVYYCERHGGDGYWYFDVWGQGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 13 - protein, 473 aa, synthetic construct
MDPKGSLSWRILLFLSLAFELSYGEVQLVESGGGLVKPGGSLRLSCAASGFTFSRYGMSWVRQAPGKGLEWVSTISSGGSYTYYTDSVKGRFTISRDNAKNSLYLQMNSLRAEDTAVYYCERHGGDGYWYFDVWGQGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 14 - protein, 473 aa, synthetic construct
MDPKGSLSWRILLFLSLAFELSYGEVQLVESGGGLVQPGRSLRLSCTASGFTFSRYGMSWVRQAPGKGLEWVATISSGGSYTYYTDSVKGRFTISRDNAKNILYLQMNSLKTEDTAVYYCERHGGDGYWYFDVWGQGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 15 - protein, 239 aa, synthetic construct
METDTLLLWVLLLWVPGSTGDVVMTQSPLSLPVTLGQPASISCRSSQSLLDSDGKTFLNWLQQRPGQSPRRLIYLVSKLDSGVPDRFSGSGSGTDFTLKISRVEAEDVGVYYCWQGTHFPYTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 16 - protein, 239 aa, synthetic construct
METDTLLLWVLLLWVPGSTGDVVMTQSPLSLPVTLGQPASISCRSSQSLLDSDGKTFLNWLQQRPGQSPRRLIYLVSKRDSGVPDRFSGSGSGTDFTLKISRVEAEDVGVYYCWQGTHFPYTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 17 - protein, 239 aa, synthetic construct
METDTLLLWVLLLWVPGSTGDVVMTQSPDSLAVSLGERATINCKSSQSLLDSDGKTFLNWLQQKPGQPPKRLIYLVSKLDSGVPDRFSGSGSGTDFTLTISSLQAEDVAVYYCWQGTHFPYTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 18 - protein, 233 aa, synthetic construct
METDTLLLWVLLLWVPGSTGDIQMTQSPSSLSASVGDRVTITCRASQDISNYLNWYQQKPGKAVKLLIYYTSRLHSGVPSRFSGSGSGTDYTLTISSLQPEDFATYFCQQGNPLRTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 19 - protein, 233 aa, synthetic construct
METDTLLLWVLLLWVPGSTGDIQMTQSPSSLSASVGDRVTITCRASQDISNYLNWYQQKPGKAVKLLIYYTSRLHSGVPSRFSGSGSGTDYTLTISSLQPEDFATYYCQQGNPLRTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 20 - protein, 233 aa, synthetic construct
METDTLLLWVLLLWVPGSTGDIVMTQSPATLSLSPGERATLSCRASQDISNYLNWYQQKPGQAVRLLIYYTSRLHSGIPARFSGSGSGTDYTLTISSLEPEDFAVYFCQQGNPLRTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 21 - protein, 473 aa, synthetic construct
  VARIANT 27: note = Any amino acid, such as Q or K
  VARIANT 30: note = Any amino acid, such as E or Q
  VARIANT 33: note = Any amino acid, such as G or A
  VARIANT 34: note = Any amino acid, such as G or E
  VARIANT 35: note = Any amino acid, such as L or V
  VARIANT 36: note = Any amino acid, such as V or K
  VARIANT 37: note = Any amino acid, such as Q or K, or absent
  VARIANT 40: note = Any amino acid, such as R, G or A
  VARIANT 42: note = Any amino acid, such as L or V
  VARIANT 43: note = Any amino acid, such as R or K
  VARIANT 44: note = Any amino acid, such as L or V
  VARIANT 47: note = Any amino acid, such as T, A or K
  VARIANT 54: note = Any amino acid, such as S or R, or absent
  VARIANT 55: note = Any amino acid, such as R or T, or absent
  VARIANT 57: note = Any amino acid, such as G or A, or absent
  VARIANT 67: note = Any amino acid, such as K or Q
  VARIANT 68: note = Any amino acid, such as G or R
  VARIANT 73: note = Any amino acid, such as A or S
  VARIANT 76: note = Any amino acid, such as S or G, or absent
  VARIANT 78: note = Any amino acid, such as G or D, or absent
  VARIANT 79: note = Any amino acid, such as G or R, or absent
  VARIANT 80: note = Any amino acid, such as S or R, or absent
  VARIANT 81: note = Any amino acid, such as Y or H, or absent
  VARIANT 85: note = Any amino acid, such as T or P, or absent
  VARIANT 87: note = Any amino acid, such as S or K
  VARIANT 88: note = Any amino acid, such as V or F
  VARIANT 89: note = Any amino acid, such as K or Q
  VARIANT 92: note = Any amino acid, such as F or V
  VARIANT 95: note = Any amino acid, such as S or T
  VARIANT 99: note = Any amino acid, such as A or S
  VARIANT 102: note = Any amino acid, such as I, S or T
  VARIANT 105: note = Any amino acid, such as L or M
  VARIANT 106: note = Any amino acid, such as Q or E
  VARIANT 107: note = Any amino acid, such as M or L
  VARIANT 108: note = Any amino acid, such as N or S
  VARIANT 111: note = Any amino acid, such as K or R
  VARIANT 112: note = Any amino acid, such as T, A or S
  VARIANT 121: note = Any amino acid, such as E or V, or absent
  VARIANT 122: note = Any amino acid, such as R or G, or absent
  VARIANT 123: note = Any amino acid, such as H or P, or absent
  VARIANT 124: note = Any amino acid, such as G or Y, or absent
  VARIANT 125: note = Any amino acid, such as G or D, or absent
  VARIANT 126: note = Any amino acid, such as D or G, or absent
  VARIANT 127: note = Any amino acid, such as G or Y, or absent
  VARIANT 129: note = Any amino acid, such as W or G, or absent
  VARIANT 130: note = Any amino acid, such as Y or E, or absent
  VARIANT 133: note = Any amino acid, such as V or Y, or absent
  VARIANT 139: note = Any amino acid, such as M or L, or absent
MDPKGSLSWRILLFLSLAFELSYGEVXLVXSGXXXXXPGXSXXXSCXASGFTFXXYXMSWVRQAPGXXLEWVXTIXSXXXXTYYXDXXXGRXTIXRDNXKNXLYXXXXSLXXEDTAVYYCXXXXXXXYXXFDXWGQGTXVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 22 - protein, 239 aa, synthetic construct
  VARIANT 22: note = Any amino acid, such as V or I, or absent
  VARIANT 23: note = Any amino acid, such as V or Q
  VARIANT 29: note = Any amino acid, such as L, D, S, or A, or absent
  VARIANT 30: note = Any amino acid, such as S or T
  VARIANT 32: note = Any amino acid, such as S, P, or A
  VARIANT 33: note = Any amino acid, such as V, A, or L
  VARIANT 34: note = Any amino acid, such as S or T
  VARIANT 35: note = Any amino acid, such as L, V, or P
  VARIANT 37: note = Any amino acid, such as Q, E, or D, or absent
  VARIANT 38: note = Any amino acid, such as R or P
  VARIANT 39: note = Any amino acid, such as A or V
  VARIANT 40: note = Any amino acid, such as T or S
  VARIANT 41: note = Any amino acid, such as I or L
  VARIANT 42: note = Any amino acid, such as S, N, or T
  VARIANT 44: note = Any amino acid, such as R or K
  VARIANT 45: note = Any amino acid, such as S or A, or absent
  VARIANT 48: note = Any amino acid, such as S or D, or absent
  VARIANT 49: note = Any amino acid, such as L or I, or absent
  VARIANT 50: note = Any amino acid, such as L or S, or absent
  VARIANT 51: note = Any amino acid, such as D or N, or absent
  VARIANT 52: note = Any amino acid, such as S or Y, or absent
  VARIANT 53: note = Any amino acid, such as D or L, or absent
  VARIANT 54: note = Any amino acid, such as G or N, or absent
  VARIANT 55: note = Any amino acid, such as K or W, or absent
  VARIANT 56: note = Any amino acid, such as T or Y, or absent
  VARIANT 57: note = Any amino acid, such as F or Q, or absent
  VARIANT 61: note = Any amino acid, such as L, or absent
  VARIANT 64: note = Any amino acid, such as K or R
  VARIANT 67: note = Any amino acid, such as Q or K
  VARIANT 68: note = Any amino acid, such as A, S, or P
  VARIANT 69: note = Any amino acid, such as P or V, or absent
  VARIANT 70: note = Any amino acid, such as R or K, or absent
  VARIANT 71: note = Any amino acid, such as R or L, or absent
  VARIANT 75: note = Any amino acid, such as L or Y, or absent
  VARIANT 76: note = Any amino acid, such as V or T, or absent
  VARIANT 78: note = Any amino acid, such as K or R, or absent
  VARIANT 79: note = Any amino acid, such as L or R
  VARIANT 80: note = Any amino acid, such as D or H, or absent
  VARIANT 83: note = Any amino acid, such as V or I
  VARIANT 85: note = Any amino acid, such as D, S, or A
  VARIANT 96: note = Any amino acid, such as F or Y, or absent
  VARIANT 99: note = Any amino acid, such as T or K
  VARIANT 102: note = Any amino acid, such as S or R
  VARIANT 103: note = Any amino acid, such as L or V
  VARIANT 104: note = Any amino acid, such as E or Q, or absent
  VARIANT 105: note = Any amino acid, such as A or P, or absent
  VARIANT 108: note = Any amino acid, such as V or F, or absent
  VARIANT 109: note = Any amino acid, such as A or G
  VARIANT 110: note = Any amino acid, such as V or T
  VARIANT 112: note = Any amino acid, such as Y or F
  VARIANT 114: note = Any amino acid, such as W or Q, or absent
  VARIANT 117: note = Any amino acid, such as T or N, or absent
  VARIANT 118: note = Any amino acid, such as H or P, or absent
  VARIANT 119: note = Any amino acid, such as F or L, or absent
  VARIANT 120: note = Any amino acid, such as P or R, or absent
  VARIANT 121: note = Any amino acid, such as Y, or absent
  VARIANT 125: note = Any amino acid, such as Q or G, or absent
  VARIANT 129: note = Any amino acid, such as L or V, or absent
METDTLLLWVLLLWVPGSTGDXXMTQSPXXLXXXXGXXXXXXCXXSQXXXXXXXXXXLNWXQQXPGXXXXXLIYXXSXXXSGXPXRFSGSGSGTDXTLXISXXXXEDXXXYXCXQGXXXXXTFGXGTKXEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 23 - protein, 330 aa, Homo sapiens [sequence omitted]

SEQ ID NO: 24 - protein, 473 aa, synthetic construct
  VARIANT 27: note = K or Q
  VARIANT 30: note = Q or E
  VARIANT 33: note = A or G
  VARIANT 34: note = E or G
  VARIANT 35: note = V or L
  VARIANT 36: note = K or V
  VARIANT 37: note = K or Q
  VARIANT 40: note = A or G
  VARIANT 42: note = V or L
  VARIANT 43: note = K or R
  VARIANT 44: note = V or L
  VARIANT 47: note = K or A
  VARIANT 67: note = Q or K
  VARIANT 68: note = R or G
  VARIANT 87: note = K or S
  VARIANT 88: note = F or V
  VARIANT 89: note = Q or K
  VARIANT 92: note = V or F
  VARIANT 95: note = T or S
  VARIANT 99: note = A or S
  VARIANT 105: note = M or L
  VARIANT 106: note = E or Q
  VARIANT 107: note = L or M
  VARIANT 108: note = S or N
  VARIANT 112: note = S or A
MDPKGSLSWRILLFLSLAFELSYGEVXLVXSGXXXXXPGXSXXXSCXASGFTFRTYAMSWVRQAPGXXLEWVATIGSDRRHTYYPDXXXGRXTIXRDNXKNTLYXXXXSLRXEDTAVYYCVGPYDGYYGEFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 25 - protein, 239 aa, synthetic construct
  VARIANT 29: note = D or L
  VARIANT 32: note = A or P
  VARIANT 34: note = S or T
  VARIANT 37: note = E or Q
  VARIANT 38: note = R or P
  VARIANT 40: note = T or S
  VARIANT 42: note = N or S
  VARIANT 44: note = K or R
  VARIANT 64: note = K or R
  VARIANT 68: note = P or S
  VARIANT 70: note = K or R
  VARIANT 79: note = L or R
  VARIANT 99: note = T or K
  VARIANT 102: note = S or R
  VARIANT 103: note = L or V
  VARIANT 104: note = Q or E
  VARIANT 109: note = A or G
METDTLLLWVLLLWVPGSTGDVVMTQSPXSLXVXLGXXAXIXCXSSQSLLDSDGKTFLNWLQQXPGQXPXRLIYLVSKXDSGVPDRFSGSGSGTDFTLXISXXXAEDVXVYYCWQGTHFPYTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 26 - protein, 4 aa, synthetic construct
GPSL

SEQ ID NO: 27 - skipped

SEQ ID NO: 28 - protein, 4 aa, synthetic construct
PSLK

SEQ ID NO: 29 - protein, 5 aa, synthetic construct
GPSLK

SEQ ID NO: 30 - protein, 4 aa, synthetic construct
SLKL

SEQ ID NO: 31 - protein, 215 aa, Mus musculus [sequence omitted]

SEQ ID NO: 32 - protein, 215 aa, Homo sapiens
MGKGDPKKPRGKMSSYAFFVQTCREEHKKKHPDASVNFSEFSKKCSERWKTMSAKEKGKFEDMAKADKARYEREMKTYIPPKGETKKKFKDPNAPKRPPSAFFLFCSEYRPKIKGEHPGLSIGDVAKKLGEMWNNTAADDKQPYEKKAAKLKEKYEKDIAAYRAKGKPDAAKKGVVKAEKSKKKKEEEEDEEDEEDEEEEEDEEDEDEEEDDDDE

SEQ ID NO: 33 - protein, 384 aa, Homo sapiens
APTKAPDVFPIISGCRHPKDNSPVVLACLITGYHPTSVTVTWYMGTQSQPQRTFPEIQRRDSYYMTSSQLSTPLQQWRQGEYKCVVQHTASKSKKEIFRWPESPKAQASSVPTAQPQAEGSLAKATTAPATTRNTGRGGEEKKKEKEKEEQEERETKTPECPSHTQPLGVYLLTPAVQDLWLRDKATFTCFVVGSDLKDAHLTWEVAGKVPTGGVEEGLLERHSNGSQSQHSRLTLPRSLWNAGTSVTCTLNHPSLPPQRLMALREPAAQAPVKLSLNLLASSDPPEAASWLLCEVSGFSPPNILLMWLEDQREVNTSGFAPARPPPQPGSTTFWAWSVLRVPAPPSPQPATYTCVVSHEDSRTLLNASRSLEVSYVTDHGPMK

SEQ ID NO: 34 - protein, 326 aa, Homo sapiens
ASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDISVEWESNGQPENNYKTTPPMLDSDGSFFLYSKLTVDKSRWQQGNVFSCSVMHEALHNHYTQKSLSLSPGK

SEQ ID NO: 35 - protein, 377 aa, Homo sapiens
ASTKGPSVFPLAPCSRSTSGGTAALGCLVKDYFPEPVTVSWNSGALTSGVHTFPAVLQSSGLYSLSSVVTVPSSSLGTQTYTCNVNHKPSNTKVDKRVELKTPLGDTTHTCPRCPEPKSCDTPPPCPRCPEPKSCDTPPPCPRCPEPKSCDTPPPCPRCPAPELLGGPSVFLFPPKPKDTLMISRTPEVTCVVVDVSHEDPEVQFKWYVDGVEVHNAKTKPREEQYNSTFRVVSVLTVLHQDWLNGKEYKCKVSNKALPAPIEKTISKTKGQPREPQVYTLPPSREEMTKNQVSLTCLVKGFYPSDIAVEWESSGQPENNYNTTPPMLDSDGSFFLYSKLTVDKSRWQQGNIFSCSVMHEALHNRFTQKSLSLSPGK

SEQ ID NO: 36 - protein, 452 aa, Homo sapiens
GSASAPTLFPLVSCENSPSDTSSVAVGCLAQDFLPDSITLSWKYKNNSDISSTRGFPSVLRGGKYAATSQVLLPSKDVMQGTDEHVVCKVQHPNGNKEKNVPLPVIAELPPKVSVFVPPRDGFFGNPRKSKLICQATGFSPRQIQVSWLREGKQVGSGVTTDQVQAEAKESGPTTYKVTSTLTIKESDWLGQSMFTCRVDHRGLTFQQNASSMCVPDQDTAIRVFAIPPSFASIFLTKSTKLTCLVTDLTTYDSVTISWTRQNGEAVKTHTNISESHPNATFSAVGEASICEDDWNSGERFTCTVTHTDLPSPLKQTISRPKGVALHRPDVYLLPPAREQLNLRESATITCLVTGFSPADVFVQWMQRGQPLSPEKYVTSAPMPEPQAPGRYFAHSILTVSEEEWNTGETYTCVAHEALPNRVTERTVDKSTGKPTLYNVSLVMSDTAGTCY

SEQ ID NO: 37 - protein, 327 aa, Homo sapiens
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 38 - protein, 353 aa, Homo sapiens
ASPTSPKVFPLSLCSTQPDGNVVIACLVQGFFPQEPLSVTWSESGQGVTARNFPPSQDASGDLYTTSSQLTLPATQCLAGKSVTCHVKHYTNPSQDVTVPCPVPSTPPTPSPSTPPTPSPSCCHPRLSLHRPALEDLLLGSEANLTCTLTGLRDASGVTFTWTPSSGKSAVQGPPERDLCGCYSVSSVLPGCAEPWNHGKTFTCTAAYPESKTPLTATLSKSGNTFRPEVHLLPPPSEELALNELVTLTCLARGFSPKDVLVRWLQGSQELPREKYLTWASRQEPSQGTTTFAVTSILRVAAEDWKKGDTFSCMVGHEALPLAFTQKTIDRLAGKPTHVNVSVVMAEVDGTCY

SEQ ID NO: 39 - protein, 340 aa, Homo sapiens
ASPTSPKVFPLSLDSTPQDGNVVVACLVQGFFPQEPLSVTWSESGQNVTARNFPPSQDASGDLYTTSSQLTLPATQCPDGKSVTCHVKHYTNPSQDVTVPCPVPPPPPCCHPRLSLHRPALEDLLLGSEANLTCTLTGLRDASGATFTWTPSSGKSAVQGPPERDLCGCYSVSSVLPGCAQPWNHGETFTCTAAHPELKTPLTANITKSGNTFRPEVHLLPPPSEELALNELVTLTCLARGFSPKDVLVRWLQGSQELPREKYLTWASRQEPSQGTTTFAVTSILRVAAEDWKKGDTFSCMVGHEALPLAFTQKTIDRMAGKPTHVNVSVVMAEVDGTCY

SEQ ID NO: 40 - protein, 106 aa, Homo sapiens
TVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 41 - DNA, 34 bp, synthetic construct
  source 1..34: mol_type = other DNA
gcgtgcatatgaacaaagcagagctcattgacgt

SEQ ID NO: 42 - DNA, 35 bp, synthetic construct
  source 1..35: mol_type = other DNA
cgtggctcttccgcacgctttgcgaccccgccgag

SEQ ID NO: 43 - protein, 41 aa, synthetic construct
  VARIANT 21: note = Any amino acid
FLEEIRLSLESGQDVKLSGFXTLSAKEIENMVKDILEFISQ

SEQ ID NO: 44 - protein, 215 aa, Mus musculus [sequence omitted]

SEQ ID NO: 45 - protein, 215 aa, Homo sapiens; identical to SEQ ID NO: 32

SEQ ID NO: 46 - protein, 10 aa, synthetic construct
PPKGETKKKF

SEQ ID NO: 47 - protein, 87 aa, Homo sapiens
KDPNAPKRPPSAFFLFSSEYRPKIKGEHPGLSIGDVAKKLGEMWNNTAADDKQPYEKKAEKLKEKYEKDIAAYRAKGKPDAAKKGVV

SEQ ID NO: 48 - protein, 97 aa, Homo sapiens
PPKGETKKKFKDPNAPKRPPSAFFLFSSEYRPKIKGEHPGLSIGDVAKKLGEMWNNTAADDKQPYEKKAEKLKEKYEKDIAAYRAKGKPDAAKKGVV

SEQ ID NO: 49 - protein, 21 aa, Homo sapiens
NFELRDKSSRPGRNPKTGDVV

SEQ ID NO: 50 - protein, 21 aa, Homo sapiens
SLHHRQPRLGRNPKTGDSVNL

SEQ ID NO: 51 - protein, 137 aa, Escherichia coli
MSEALKILNNIRTLRAQARECTLETLEEMLEKLEVVVNERREEESAAAAEVEERTRKLQQYREMLIADGIDPNELLNSLAAVKSGTKAKRAQRPAKYSYVDENGETKTWTGQGRTPAVIKKAMDEQGKSLDDFLIKQ

SEQ ID NO: 52 - protein, 134 aa, Haemophilus influenzae
MNELVRGLTNLRSLRAAVRELTLEQAENALEKLQTAIEEKRANEAELIKAETERKERLAKYKELMEKEGITPEELHKIFGTKTVSIQAKRAPRPAKYAFIDENGEHKTWTGQGRTPRPIQNALNKGKSLSDFEI

SEQ ID NO: 53 - protein, 134 aa, Mycobacterium tuberculosis
MPDPQDRPDSEPSDASTPPAKKLPAKKAAKKAPARKTPAKKAPAKKTPAKGAKSAPPKPAEAPVSLQQRIETNGQLAAAAKDAAAQAKSTVEGANDALARNASVPAPSHSPVPLIVAVTLSLLALLLIRQLRRR

SEQ ID NO: 54 - protein, 134 aa, Streptococcus pneumoniae
MNELVRSLTNLRSLRAAVRELTLEQAENALEKLQTAIEEKRANEAELIKAETERKERLAKYKELMEKEGITPEELHEIFGTKTVSIRAKRPPRPAKYAFIDENGEHKTWTGQGRTPRPIQNALNKGKSLSDFEI

SEQ ID NO: 55 - protein, 135 aa, Klebsiella pneumoniae
MSEALKILNNIRTLRAQARECTLETLEEMLEKLEVVVNERREEENAAAAEIEERTRKLQQYREMLIADGIDPNELLSTMAAVKAGTKTKRAARPAKYSYVDENGETKTWTGQGRTPAVIKKAMDEQGKSLDDFLI

SEQ ID NO: 56 - protein, 120 aa, Pseudomonas aeruginosa
MSLINEYRNTEQTIKELQARLASLQQDGRMKAELEFDTKLRALMSEYNKSLRDVIILLDPQAQNRSSKTPPTSGRRERQLKRYLNPNTSEVVETKGGNHKILKEWKTQFGADVVESWLQS

SEQ ID NO: 57 - protein, 90 aa, Escherichia coli
MNKSQLIDKIAAGADISKAAAGRALDAIIASVTESLKEGDDVALVGFGTFAVKERAARTGRNPQTGKEITIAAAKVPSFRAGKALKDAVN

SEQ ID NO: 58 - protein, 7 aa, synthetic construct
GFTFRTY

SEQ ID NO: 59 - protein, 6 aa, synthetic construct
GSDRRH

SEQ ID NO: 60 - protein, 13 aa, synthetic construct
VGPYDGYYGEFDY

SEQ ID NO: 61 - protein, 11 aa, synthetic construct
QSLLDSDGKTF

SEQ ID NO: 62 - protein, 7 aa, synthetic construct
WQGTHFP

SEQ ID NO: 63 - protein, 8 aa, synthetic construct
GFTFSRYG

SEQ ID NO: 64 - protein, 8 aa, synthetic construct
ISSGGSYT

SEQ ID NO: 65 - protein, 13 aa, synthetic construct
ERHGGDGYWYFDV

SEQ ID NO: 66 - protein, 9 aa, synthetic construct
WQGTHFPYT

(19) United States
(12) Patent Application Publication
(10) Pub. No.: US 2026/0176328 A1
(43) Pub. Date: Jun. 25, 2026

(54) ANTIBODY COMPOSITIONS AND METHODS FOR DISRUPTING NONTUBERCULOUS MYCOBACTERIA BIOFILMS

(71) Applicant: Research Institute of Nationwide Children’s Hospital, Columbus, OH (US)

(72) Inventors: Steven D. Goodman, Columbus, OH (US); Lauren O. Bakaletz, Columbus, OH (US)

(21) Appl. No.: 19/128,750

(22) PCT Filed: Nov. 14, 2023
(86) PCT No.: PCT/US2023/037277

§ 371 Date: May 9, 2025

Related U.S. Application Data
(60) Provisional application No. 63/425,261, filed on Nov. 14, 2022.

(51) Int. Cl. C07K 14/78; A61K 9/00; A61K 38/00; A61K 39/395; A61K 45/06; C07K 16/18

(52) U.S. Cl. CPC C07K 14/78; A61K 9/0019; A61K 38/00; A61K 39/3955; A61K 45/06; C07K 16/18; C07K 2317/33; C07K 2317/565

(57) ABSTRACT

Provided herein are methods for preventing or treating an infection caused by a Nontuberculous mycobacterium (NTM) species in a subject comprising administering to the subject an effective amount of an antibody or an antigen-binding fragment thereof that binds to a tip region of a DNABII peptide. Also provided in are methods for sensitizing a biofilm to an antibiotic agent, wherein the biofilm comprises a Nontuberculous mycobacterium (NTM) species, the method comprising contacting the biofilm with an antibody or an antigen-binding fragment thereof that binds to a tip region of a DNABII peptide.

### CROSS-REFERENCE TO RELATED PATENT APPLICATION

This application is a U.S. National Stage Application under 35 U.S.C. § 371 of International Patent Application No. PCT/US2023/037277, filed Nov. 14, 2023, which claims the benefit under 35 U.S.C. § 119 (e) of U.S. Provisional Application No. 63/425,261, filed Nov. 14, 2022, the entire content of which is incorporated herein by reference.

### SEQUENCE LISTING

This application contains a Sequence Listing which has been submitted electronically in XML format and is hereby incorporated by reference in its entirety. Said XML copy, created on Jan. 8, 2024, is named 106887-9290_SL.xml and is 141,162 bytes in size.

### BACKGROUND

Nontuberculous mycobacteria (NTM), distantly related to Mycobacterium tuberculosis, cause chronic infections in people with muco-obstructive pulmonary diseases, such as chronic obstructive pulmonary disease (COPD), primary ciliary dyskinesia, or CF. NTM are classified as slow (e.g., Mycobacterium avium) or rapidly growing (e.g., Mycobacterium abscessus), and in addition to genotypic/phenotypic variability, NTM prevalence varies with underlying lung diseases. People with CF (PwCF) are highly vulnerable to lung infection and NTM are prevalent in both adults and children in the United States and Europe, with NTM prevalence increasing by 5% annually. One in 5 PwCF are culture positive for NTM with acquisition associated with geographic region, increasing age, and NTM species.

Established NTM infections are extremely difficult to treat and require prolonged antibiotic therapy. Recommended treatment for PwCF who are culture-positive for M. avium complex (M. avium, M. intracellulare and M. chimaera) is an up to 17-months-long regimen of oral and intravenous antibiotics dependent on disease severity. M. abscessus presents an even greater challenge with treatment regimens typically involving oral, intravenous, and inhaled antibiotics for up to 18-24 months. Despite these intense regimens, failure rate is high with 50%-60% of people unable to both transition from NTM-positive to-negative sputum cultures and maintain this conversion for over 12 months. Critically, 30-60% of patients had to discontinue at least one of the prescribed antibiotics due to considerable treatment sequelae such as drug-related toxicity (e.g., nephrotoxicity or auditory-vestibular toxicity).

Reasons for this difficulty treating NTM infections include low drug uptake due to thick, hydrophobic mycobacterial cell walls, export of drugs by efflux pumps, development of antibiotic resistance, and biofilm formation by NTM. Biofilms are aggregated bacterial communities embedded in an extracellular polymeric substance (EPS) of proteins, carbohydrates and extracellular DNA (eDNA). They are phenotypically distinct from planktonic bacteria and found in the lung and/or sputum of PwCF or COPD. Biofilm-resident bacteria are well protected from antibiotics, chemical agents, mechanical stress, and immune effectors through diverse mechanisms. Further, they can tolerate antibiotics at many times the concentration required to kill their planktonic counterparts. Canonically, clinical isolates are considered more virulent and more representative of disease-causative agents than their lab-passaged counterparts, as clinical isolates of M. abscessus display increased aggregation as well as intracellular survival and further, they induce greater inflammation relative to the lab-passaged reference strain, M. abscessus 19977. Thereby, consideration of testing clinical isolates in addition to lab-passaged strains is highly recommended. Novel and more effective approaches to combat recalcitrant NTM infections are urgently needed. This disclosure satisfies this need and provides related advantages as well.

### SUMMARY OF THE DISCLOSURE

Applicant has discovered that an antibody or an antigen-binding fragment thereof that binds to a tip region of a DNABII peptide can surprisingly disrupt or prevent biofilms formed by or containing a Nontuberculous mycobacterium (NTM) species. Applicant has also discovered that such anti-DNABII tip antibodies reduce antibiotic resistance in NTM biofilms and reduce the dose of antibiotic necessary to treat such biofilms and the infection.

An aspect of the disclosure is directed to a method for preventing or treating an infection caused by a Nontuberculous mycobacterium (NTM) species in a subject comprising, or consisting essentially, or consisting of administering to the subject an effective amount of an antibody or an antigen-binding fragment thereof that binds to a tip region of a DNABII peptide.

Another aspect of the disclosure is directed to a method for sensitizing a biofilm or a microorganism in the biofilm to an antibiotic agent, wherein the biofilm comprises, or consisting essentially, or consisting of a Nontuberculous mycobacterium (NTM) species, the method comprising, or consisting essentially, or consisting of contacting the biofilm with an antibody or an antigen-binding fragment thereof that binds to a tip region of a DNABII peptide. The contacting can be in vitro or in vivo. When performed in vivo, the method provides a method for sensitizing a biofilm produced by a Nontuberculous mycobacterium (NTM) species to an antibiotic agent in a subject in need thereof, the method comprising, or consisting essentially, or consisting of administering to the subject an antibody or an antigen-binding fragment thereof that binds to a tip region of a DNABII peptide. In one aspect, the method reduces the dose of antibiotic necessary to treat such biofilms and accompanying infection.

In another aspect, provided is a method to disrupt a biofilm produced by a nontuberculous mycobacteria (NTM), e.g., Mycobacterium abscessus (Mab) or Mycobacterium avium (Mav). The method comprises or consists essentially of, or yet further consists of contacting the biofilm produced by a nontuberculous mycobacteria (NTM), e.g., Mycobacterium abscessus (Mab) or Mycobacterium avium (Mav). with one or more of an antibody, a fragment thereof, a polypeptide, or a CDR as disclosed herein. In one embodiment, the antibody, fragment thereof, polypeptide, or CDR binds a tip region of a DNABII peptide (including but not limited to: a tip region of IHF or HU, a tip region of IHFA or IHFB, the tip-chimeric peptide IhfA5-mIhfB4_{NTHI}). In a further embodiment, the antibody, fragment thereof, polypeptide, or CDR binds the tip-chimeric peptide IhfA5-mIhfB4_{NTHI}. In yet a further embodiment, the contacting is or.

In one aspect, provided is a method for detecting a nontuberculous mycobacteria (NTM), e.g., Mycobacterium abscessus (Mab) or Mycobacterium avium (Mav). microbial infection that produces a biofilm in a subject. The method comprises, or alternatively consists of, or yet further consists of contacting one or more of the antibody, fragment thereof, polypeptide, or CDR as disclosed herein with a biological sample suspected of comprising the biofilm and isolated from the subject and detecting the binding of the antibody, fragment thereof, polypeptide, or CDR to any biofilm in the sample. The amount can be determined by the treating physician or veterinarian, e.g., an effective amount for the subject. In one embodiment, the antibody, fragment thereof, polypeptide or CDR binds a tail or tip region of a DNABII peptide (including but not limited to: a tip region of IHF or HU, a tip region of IHFA or IHFB, the tip-chimeric peptide IhfA5-mIhfB4_{NTHI}, a tail region of IHF or HU, a tail region of IHFA or IHFB, and/or the tail-chimeric peptide IhfA3-IhfB2_{NTHI}). In a further embodiment, the contacting is or.

In another aspect, provided is a method for screening subjects having a biofilm-producing nontuberculous mycobacterial infection (NTM), e.g., Mycobacterium abscessus (Mab) or Mycobacterium avium (Mav), the method comprising or alternatively consisting of, or yet further consisting of, contacting one or more of an antibody, a fragment thereof, a polypeptide, or a CDR as disclosed herein with a biological sample comprising the biofilm and isolated from the subject, and detecting the binding of the antibody, fragment thereof, polypeptide, or CDR to any biofilm in the sample. In one embodiment, the antibody, fragment thereof, polypeptide or CDR binds a tail or tip region of a DNABII peptide (including but not limited to: a tip region of IHF or HU, a tip region of IHFA or IHFB, the tip-chimeric peptide IhfA5-mIhfB4_{NTHI}, a tail region of IHF or HU, a tail region of IHFA or IHFB, and/or the tail-chimeric peptide IhfA3-IhfB2_{NTHI}). In a further embodiment, a subject detected with the binding is selected for administration with one or more of an antibody, a fragment thereof, a polypeptide, or a CDR as disclosed herein, and/or one or more of a polynucleotide or a vector encoding the antibody, fragment thereof, polypeptide or CDR, wherein the antibody, fragment thereof, polypeptide, or CDR binds a tip region of a DNABII peptide (including but not limited to: a tip region of IHF or HU, a tip region of IHFA or IHFB, and/or the tip-chimeric peptide IhfA5-mIhfB4_{NTHI}). The amount can be determined by the treating physician or veterinarian, e.g., an effective amount for the subject. In yet a further embodiment, the contacting is or.

Provided herein are methods to detect a biofilm produced by a nontuberculous mycobacterial infection (NTM), e.g., Mycobacterium abscessus (Mab) or Mycobacterium avium (Mav), in a subject by administering to the subject one or more of the antibody or the fragment thereof as disclosed herein, and detecting any binding of the antibody, the fragment thereof, polypeptide or CDR as disclosed herein to the biofilm. In one aspect, the antibody, fragment thereof, polypeptide or CDR binds a tail or tip region of a DNABII peptide (including but not limited to: a tip region of IHF or HU, a tip region of IHFA or IHFB, the tip-chimeric peptide IhfA5-mIhfB4_{NTHI}, a tail region of IHF or HU, a tail region of IHFA or IHFB, and/or the tail-chimeric peptide IhfA3-IhfB2_{NTHI}). In another embodiment, the method further comprises detecting binding of the antibody, fragment thereof, polypeptide or CDR to the biofilm.

In one aspect, methods to prevent or disrupt a biofilm produced by a nontuberculous mycobacterial infection (NTM), e.g., Mycobacterium abscessus (Mab) or Mycobacterium avium (Mav), in a subject are provided, comprising, or alternatively consisting of, or yet further consisting of, administering to the subject one or more of the antibody, fragment thereof, polypeptide, or CDR as disclosed herein, and/or one or more of a polynucleotide or a vector encoding the antibody, fragment thereof, polypeptide or CDR, wherein such binds to a tip region of a DNABII peptide (including but not limited to: a tip region of IHF or HU, a tip region of IHFA or IHFB, and/or the tip-chimeric peptide IhfA5-mIhfB4_{NTHI}). The amount can be determined by the treating physician or veterinarian, e.g., an effective amount for the subject. In one embodiment, the method further comprises detecting a biofilm by contacting one or more of an antibody, a fragment thereof, a polypeptide, or a CDR as disclosed herein with a sample suspected of containing a biofilm, and detecting the binding of the biofilm and the antibody, fragment thereof, polypeptide, or CDR, and wherein the antibody, fragment thereof, polypeptide, or CDR binds to the tip region or the tail region of a DNABII peptide (including but not limited to: a tip region of IHF or HU, a tip region of IHFA or IHFB, the tip-chimeric peptide IhfA5-mIhfB4_{NTHI}, a tail region of IHF or HU, a tail region of IHFA or IHFB, and/or the tail-chimeric peptide IhfA3-IhfB2_{NTHI}).

In another aspect, methods for inhibiting, preventing or treating a microbial infection produced by a nontuberculous mycobacterial infection (NTM), e.g., Mycobacterium abscessus (Mab) or Mycobacterium avium (Mav), are provided, comprising, or alternatively consisting of, or yet further consisting of, administering to the subject one or more of the antibody, fragment thereof, polypeptide, or CDR as disclosed herein, and/or of one or more of a polynucleotide or a vector encoding the antibody, fragment thereof, polypeptide or CDR, wherein such binds to a tip region of a DNABII peptide (including but not limited to: a tip region of IHF or HU, a tip region of IHFA or IHFB, and/or the tip-chimeric peptide IhfA5-mIhfB4_{NTHI}). The amount can be determined by the treating physician or veterinarian, e.g., an effective amount for the subject. In one embodiment, the method further comprises detecting a biofilm by contacting one or more of an antibody, a fragment thereof, a polypeptide, or a CDR as disclosed herein with a sample suspected of containing a biofilm, and detecting the binding of the biofilm and the antibody, fragment thereof, polypeptide, or CDR, and wherein the antibody, fragment thereof, polypeptide, or CDR binds to the tip region or the tail region of a DNABII peptide (including but not limited to: a tip region of IHF or HU, a tip region of IHFA or IHFB, the tip-chimeric peptide IhfA5-mIhfB4_{NTHI}, a tail region of IHF or HU, a tail region of IHFA or IHFB, and/or the tail-chimeric peptide IhfA3-IhfB2_{NTHI}). In a further aspect, an effective amount of amikacin and/or azithromycin is administered to the subject, prior to, concurrently or subsequent to the one or more of the antibody, fragment thereof, polypeptide, or CDR as disclosed herein, and/or of one or more of a polynucleotide or a vector encoding the antibody, fragment thereof, polypeptide or CDR, wherein such binds to a tip region of a DNABII peptide (including but not limited to: a tip region of IHF or HU, a tip region of IHFA or IHFB, and/or the tip-chimeric peptide IhfA5-mIhfB4_{NTHI}). The amount can be determined by the treating physician or veterinarian, e.g., an effective amount for the subject. In one aspect, the subject is a human patient that is suffering from or predisposed to developing cystic fibrosis.

In yet another aspect, methods to prevent or treat a condition caused by a nontuberculous mycobacterial infection (NTM), e.g., Mycobacterium abscessus (Mab) or Mycobacterium avium (Mav) infection characterized by the formation of biofilm in a subject are provided by administering to the subject one or more of the antibody, the fragment thereof, polypeptide, or CDR as disclosed herein, and/or one or more of a polynucleotide or a vector encoding the antibody, fragment thereof, polypeptide or CDR, wherein such binds to a tip region of a DNABII peptide (including but not limited to: a tip region of IHF or HU, a tip region of IHFA or IHFB, and/or the tip-chimeric peptide IhfA5-mIhfB4_{NTHI}). The amount can be determined by the treating physician or veterinarian, e.g., an effective amount for the subject. In a further aspect, an effective amount of amikacin and/or azithromycin is administered to the subject, prior to, concurrently or subsequent to the one or more of the antibody, fragment thereof, polypeptide, or CDR as disclosed herein, and/or of one or more of a polynucleotide or a vector encoding the antibody, fragment thereof, polypeptide or CDR, wherein such binds to a tip region of a DNABII peptide (including but not limited to: a tip region of IHF or HU, a tip region of IHFA or IHFB, and/or the tip-chimeric peptide IhfA5-mIhfB4_{NTHI}). The amount can be determined by the treating physician or veterinarian, e.g., an effective amount for the subject. In one aspect, the subject is a human patient that is suffering from or predisposed to developing cystic fibrosis. In one embodiment, the method further comprises detecting a biofilm by contacting one or more of an antibody, a fragment thereof, a polypeptide, or a CDR as disclosed herein with a sample suspected of containing a biofilm, and detecting the binding of the biofilm and the antibody, fragment thereof, polypeptide, or CDR, and wherein the antibody, fragment thereof, polypeptide, or CDR binds to the tip region or the tail region of a DNABII peptide (including but not limited to: a tip region of IHF or HU, a tip region of IHFA or IHFB, the tip-chimeric peptide IhfA5-mIhfB4_{NTHI}, a tail region of IHF or HU, a tail region of IHFA or IHFB, and/or the tail-chimeric peptide IhfA3-IhfB2_{NTHI}).

Also provided is a method for conferring passive immunity in a subject suffering from an infection with a nontuberculous mycobacterial infection, e.g., Mycobacterium abscessus (Mab) or Mycobacterium avium (Mav) infection, the method comprising, or alternatively consisting essentially of, or yet further consisting of administering to the subject one or more of an antibody, fragment thereof of, polypeptide, or CDR as disclosed herein, and/or one or more of a polynucleotide or a vector encoding the antibody, fragment thereof, polypeptide or CDR, wherein the antibody, fragment, polypeptide or CDR binds to a tip region of a DNABII peptide (including but not limited to: a tip region of IHF or HU, a tip region of IHFA or IHFB, and/or the tip-chimeric peptide IhfA5-mIhfB4_{NTHI}). The amount can be determined by the treating physician or veterinarian, e.g., an effective amount for the subject. In one aspect, the subject is a human patient suffering from cystic fibrosis.

In one aspect, the therapeutic methods are combined with diagnostic methods to detect and/or monitor biofilm formation and disruption, using an antibody, a fragment thereof, a polypeptide or a CDR as disclosed herein.

Further provided are pharmaceutical compositions comprising, or consisting essentially of, or yet further consisting of as the active agents an antibody or an antigen-binding fragment thereof as described herein in combination with an effective amount of an antibiotic to treat the infection causing or having caused the biofilm as described herein, and/or an mB Box-97 polypeptide and/or a DNA-binding agent, each as described herein. In one aspect, the effective amount of the antibiotic is provided in an amount less than, the accepted MIC for the antibiotic for the treatment of the infection.

Kits also are provided. The kits comprise one or more of an antibody, an antigen-binding fragment thereof, and one or more of a polypeptide (e.g., an mB Box-97 polypeptide), a DNA-binding agent, an effective amount of an antibiotic to treat the infection or a composition as disclosed herein and optionally, instructions for use.

Applicant ‘s disclosure addresses and solves problems associated with NTM infections and NTM-containing biofilms. NTM species belong to the class Actinomycetia and are distinguished by a thick, mycolic acid-rich cell wall. NTM biofilms are especially resistant to antibiotics. Before the present disclosure, it was not known whether NTM biofilms incorporated DNABII proteins within the eDNA-rich biofilm matrix nor was it known whether antibodies generated against specific protective domains of a traditional DNABII protein (anti-DNABII tip antibodies disclosed herein) would both recognize the unique mycobacterial DNABII homologue and actively disrupt NTM biofilms. Applicant has shown that anti-DNABII tip antibodies disrupt NTM biofilms and render them sensitive to antibiotic treatment. The methods of the present disclosure enhance antibiotic sensitivity and/or immune system clearance in infected individuals. The disclosed methods also benefit patients by reducing either length of antibacterial treatment or amount of antibiotic required.

### BRIEF DESCRIPTION OF THE DRAWINGS

FIGS. 1A-1C: HuTipMab (“HuTipMab” is an antibody as described in Kurbatfinski, N. et al. Antimicrobial Agents and Chemotherapy 66.3 (2022): e01877-21 and WO 2021007260A2) recognizes isolated NTM DNA binding protein HupB (amino acid sequence as shown in SEQ ID NO: 57). (FIG. 1A) Silver-stained gel of SDS-PAGE-separated protein revealed pure protein isolation with band at anticipated location based on the following expected molecular mass of protein monomer: HupB=28 kDa. (FIG. 1B) Specificity of HuTipMab to tail-chimer peptide (negative control, as shown in SEQ ID NO: 43), HupB, and tip-chimer peptide (positive control) was determined via ELISA. Dark wells of representative image of ELISA plate indicated reactivity of HuTipMab to HupB and tip-chimer peptide. (FIG. 1C) Color developed in wells of ELISA plates was quantified via plate reader by measurement of optical density at 650 nm. Specificity of HuTipMab to HupB and tip-chimer peptide was significantly greater than to tail-chimer peptide, which showed no reactivity. Statistically significant differences in optical density are reported as ***, P≤0.001; ****, P<0.0001.

FIG. 2: Graphed mean biomass values of each treatment. HuTipMab disrupted 72 hr M. abscessus 19977 biofilms in a dose- and time-dependent manner. M. abscessus 19977 biofilms were incubated with medium alone, 5 ug human IgG (“HuIgG”), or 5, 7.5, or 10 ug HuTipMab, stained with FM 1-43FX, fixed and visualized by confocal laser scanning microscope (“CLSM”). Biomass was calculated via COMSTAT2. All treatments were incubated for 30 min or with 5 ug HuTipMab for 60 min. M. abscessus 19977 biofilms treated with HuTipMab displayed a marked relative reduction in biomass and height as compared to biofilms incubated with medium or HuIgG. There was no significant difference in biomass between wells incubated with medium alone or 5 ug HuIgG. Relative to HuIgG incubation, all wells incubated with HuTipMab were significantly reduced in biomass from 53% to 89% biofilm disruption. Statistically significant differences in biomass are reported as **, P≤0.01; ****, P<0.0001.

FIG. 3: Mean biomass values of biofilms incubated with each treatment condition represented graphically. HuTipMab-induced disruption of 2-week-old M. avium biofilms was dose- and time-dependent. Post-incubation of M. avium biofilms with medium alone, 5 ug HuIgG, or 5, 7.5, or 10 ug HuTipMab for 30 min or with 5 ug HuTipMab for 60 min), biofilms were stained with FM 1-43FX., fixed and visualized via CLSM. Displayed 3D biofilm images are representative of biofilms after each treatment. Biomass was calculated via COMSTAT2 and mean biomass values post-incubation for each treatment are in lower right portion of each image. Biomass and height of M. avium biofilms incubated with HuTipMab were notably reduced compared to that seen in wells incubated with medium alone or 5 ug HuIgG. Biomass values from wells incubated with medium alone or 5 ug HuIgG were not significantly different. However, when biofilms were incubated with HuTipMab, biomass values were significantly reduced as compared to those incubated with HuIgG alone with percent biomass disruption ranging from 51% to 80%. Statistically significant differences in biomass are reported as **, P≤0.01; ***, P≤0.001; ****, P<0.0001.

FIGS. 4A-4C: Disruption of 72 hr biofilms formed by 3 isolates of M. abscessus cultured from PwCF by HuTipMab was also dose- and time-dependent. Biofilms of M. abscessus clinical isolates 1, 2 and 3 were incubated with medium alone, 5 ug HuIgG, or 5, 7.5, or 10 ug HuTipMab for 30 min or for 60 min with 5 ug HuTipMab. Biofilms were stained with FM 1-43FX, fixed and visualized via CLSM. Biomass was calculated via COMSTAT2. Incubation with HuTipMab notably reduced biomass and height of biofilms compared to incubation with medium alone or 5 ug HuIgG. (FIG. 4A) Graphical representation of mean biomass values for biofilms formed by M. abscessus clinical isolate 1 post-incubation with each treatment condition. (FIG. 4B) Graphical representation of mean biomass values for biofilms formed by M. abscessus clinical isolate 1 post-incubation with each treatment condition. (FIG. 4C) Graphical representation of mean biomass values for biofilms formed by M. abscessus clinical isolate 1 post-incubation with each treatment condition. Disruption was significant for each HuTipMab treatment relative to treatment with HuIgG, which was never significantly different from wells treated with medium alone. Percent biomass disruption for clinical isolate 1 ranged from 58% to 91%, for clinical isolate 2 ranged from 57% to 90%, and for clinical isolate 3 ranged from 62% to 93%. Statistically significant differences in biomass are reported as *, P≤0.05; **, P≤0.01; ***, P<0.001; ****, P<0.0001.

FIGS. 5A-5C: M. abscessus 19977 NRel (NRel=newly released bacteria) were significantly more susceptible to amikacin and azithromycin. As compared to killing of planktonic, killing of NRel was significantly greater for both (FIG. 5A) amikacin and (FIG. 5B) azithromycin, notably at ¼ and ½ the MIC. Killing of planktonic was 23% and 20% respectively and killing of NRel was 61% and 42% respectively. Statistically significant differences in percent killing are reported as **, P≤0.01; ***, P≤0.001. (FIG. 5C) Graphical representation of non-biofilm associated (NBfA—also known as “planktonic”) and NRel production after treatment with an antibody against the tip region of DNABII protein (HuTipMab).

FIGS. 6A-6B: HuTipMab-induced M. avium NRel were significantly sensitized to amikacin and azithromycin. When tested against both amikacin and azithromycin, 2 antibiotics used to treat NTM in PwCF, NRel were significantly more sensitive to antibiotic killing than planktonic M. avium. (FIG. 6A) In combination with amikacin at ¼ MIC, killing of planktonic M. avium was 17% while killing of M. avium NRel was 41%. (FIG. 6B) Similarly, incubation of planktonic M. avium with 1/2 MIC of azithromycin resulted in 19% killing while incubation of M. avium NRel at the same concentration resulted in 36% killing. Statistically significant differences in percent killing are reported as *, P≤0.05; **, P≤0.01; ***, P≤0.001; ****, P<0.0001.

FIGS. 7A-7D: HuTipMab-induced NRel of M. abscessus clinical isolates 1 and 3 were significantly sensitized to killing by amikacin and azithromycin than when grown planktonically. (FIG. 7A) & (FIG. 7B) In combination with amikacin, killing of planktonic M. abscessus clinical isolate 1 was 28% whereas that of this clinical isolate's HuTipMab-induced NRel was 45%. Similarly, incubation of planktonic M. abscessus clinical isolate 1 with azithromycin resulted in 28% killing whereas killing of M. abscessus clinical isolate 1 NRel at the same concentration was 47%. (FIG. 7C) & (FIG. 7D) Killing of planktonic M. abscessus clinical isolate 3 by amikacin was 21% whereas killing of the corresponding NRel was 47%. Similarly, azithromycin killed 45% of M. abscessus clinical isolate 3 NRel at the same concentration that killed 25% of planktonic M. abscessus clinical isolate 3. Statistically significant differences in percent killing are reported as *, P≤0.05; **, P≤0.01; ***, P≤0.001; ****, P<0.0001.

### DETAILED DESCRIPTION

Unless defined otherwise, all technical and scientific terms used herein have the same meanings as commonly understood by one of ordinary skill in the art to which this invention belongs. Although any methods and materials similar or equivalent to those described herein can be used in the practice or testing of the present invention, the preferred methods, devices and materials are now described. All technical and patent publications cited herein are incorporated herein by reference in their entirety. Nothing herein is to be construed as an admission that the disclosure is not entitled to antedate such disclosure by virtue of prior disclosure.

The practice of the present disclosure will employ, unless otherwise indicated, conventional techniques of tissue culture, immunology, molecular biology, microbiology, cell biology and recombinant DNA, which are within the skill of the art. See, e.g., Sambrook and Russell eds. (2001) Molecular Cloning: A Laboratory Manual, 3^{rd} edition; the series Ausubel et al. eds. (2007) Current Protocols in Molecular Biology; the series Methods in Enzymology (Academic Press, Inc., N.Y.); MacPherson et al. (1991) PCR 1: A Practical Approach (IRL Press at Oxford University Press); MacPherson et al. (1995) PCR 2: A Practical Approach; Harlow and Lane eds. (1999) Antibodies, A Laboratory Manual; Freshney (2005) Culture of Animal Cells: A Manual of Basic Technique, 5^{th} edition; Gait ed. (1984) Oligonucleotide Synthesis; U.S. Pat. No. 4,683,195; Hames and Higgins eds. (1984) Nucleic Acid Hybridization; Anderson (1999) Nucleic Acid Hybridization; Hames and Higgins eds. (1984) Transcription and Translation; Immobilized Cells and Enzymes (IRL Press (1986)); Perbal (1984) A Practical Guide to Molecular Cloning; Miller and Calos eds. (1987) Gene Transfer Vectors for Mammalian Cells (Cold Spring Harbor Laboratory); Makrides ed. (2003) Gene Transfer and Expression in Mammalian Cells; Mayer and Walker eds. (1987) Immunochemical Methods in Cell and Molecular Biology (Academic Press, London); and Herzenberg et al. eds (1996) Weir's Handbook of Experimental Immunology.

All numerical designations, e.g., pH, temperature, time, concentration and molecular weight, including ranges, are approximations which are varied (+) or (−) by increments of 1.0 or 0.1, as appropriate, or alternatively by a variation of +/−15%, or alternatively 10%, or alternatively 5% or alternatively 2%. It is to be understood, although not always explicitly stated, that all numerical designations are preceded by the term “about”. It also is to be understood, although not always explicitly stated, that the reagents described herein are merely exemplary and that equivalents of such are known in the art.

As used in the specification and claims, the singular form “a”, “an” and “the” include plural references unless the context clearly dictates otherwise. For example, the term “a polypeptide” includes a plurality of polypeptides, including mixtures thereof.

As used herein, the term “comprising” is intended to mean that the compositions and methods include the recited elements, but do not exclude others. “Consisting essentially of” when used to define compositions and methods, shall mean excluding other elements of any essential significance to the combination for the intended use. Thus, a composition consisting essentially of the elements as defined herein would not exclude trace contaminants from the isolation and purification method and pharmaceutically acceptable carriers, such as phosphate buffered saline, preservatives, and the like. “Consisting of” shall mean excluding more than trace elements of other ingredients and substantial method steps for administering the compositions of this disclosure. Embodiments defined by each of these transition terms are within the scope of this disclosure.

As used herein, the term “comprising” is intended to mean that the peptides recited herein include the recited amino acid sequences, but do not exclude other amino acids. “Consisting essentially of” when used to define sequences, refers to a core sequence optionally surrounded by additional amino acids. Thus, a peptide consisting essentially of a sequence as defined herein would not exclude additional amino acids at the C or N terminal. In some embodiments, a peptide consisting essentially of a sequence as defined herein comprises up to ten additional amino acids (e.g., 1, 2, 3, 4, 5, 6, 7, 8, 9, or 10) at the N terminal. In some embodiments, a peptide consisting essentially of a sequence as defined herein comprises up to ten additional amino acids at the C terminal. In some embodiments, a peptide consisting essentially of a sequence as defined herein comprises up to ten additional amino acids at the N terminal and up to ten additional amino acids at the C terminal. As used for a peptide, “consisting of” a sequence means that the peptide does not contain any additional sequences. Embodiments defined by each of these transition terms are within the scope of this disclosure.

A “biofilm” intends a thin layer or an organized community of microorganisms that at times can adhere to the surface of a structure, that may be organic or inorganic, or be concentrated at an interface (usually solid/liquid), surrounded by an extracellular polymeric slime matrix that comprise polymers, such as DNA, that the microorganisms secrete and/or release. Biofilms can contain many different types of micro-organisms, e.g., bacteria, archaea, protozoa, fungi and algae. Biofilms are very resistant to microbiotics and antimicrobial agents. They live on gingival tissues, teeth, and restorations, causing caries and periodontal disease, also known as periodontal plaque disease. They also cause chronic middle ear infections. Biofilms can also form on the surface of dental implants, stents, catheter lines and contact lenses. They grow on pacemakers, heart valve replacements, artificial joints and other surgical implants. The Centers for Disease Control estimate that over 65% of nosocomial (hospital-acquired) infections are caused by biofilms. Fungal biofilms also frequently contaminate medical devices. They cause chronic vaginal infections and lead to life-threatening systemic infections in people with hobbled immune systems. Biofilms also are involved in numerous diseases. For instance, cystic fibrosis patients have Pseudomonas or Nontuberculous mycobacterium (NTM) infections that often result in antibiotic resistant biofilms.

As used herein, “treatment of a biofilm-related disorder”, also referred to herein as “for preventing or treating a bacterial biofilm “, refers to a reduction in the severity and/or duration of the disorder and/or a reduction of the severity and/or duration of symptoms from the disorder, in particular the symptoms of infection. In some embodiments, treatment results in restoration of the health of an individual. Preferably, the individual has less severe disease symptoms or for a shorter time. Disease symptoms can be monitored using convention techniques. In one aspect, the term “treatment” excludes prevention.

In some embodiments, the compositions of the present disclosure can be used for prevention or reduction of biofilm formation or growth, ex vivo, or. As used herein, “prevention or reduction of biofilm formation or growth” refers to the prevention, delay, or reduction of biofilm formation or growth, ex vivo, or. As will be understood by a skilled person, such reduction of biofilm formation or growth can slow the growth of biofilms as compared to the growth of untreated biofilms.

In some embodiments the compositions disclosed herein are useful for degrading or reducing biofilms. As used herein, “degrading or reducing biofilms” refers to the elimination, either partially or completely, of a biofilm, ex vivo, or. As will be understood by a skilled person, after such treatment, planktonic bacteria (i.e., free-living bacteria that are suspended in liquid) may still be present.

A “DNABII polypeptide or protein” intends a DNA binding protein or polypeptide that is composed of DNA-binding domains and thus have a specific or general affinity for DNA. In one aspect, they bind DNA in the minor grove. Non-limiting examples of DNABII proteins are an integration host factor (IHF) protein and a histone-like protein from E. coli strain U93 (HU). Other DNA binding proteins that can be associated with the biofilm include DPS (Genbank Accession No.: CAA49169), H-NS (Genbank Accession No.: CAA47740), Hfq (Genbank Accession No.: ACE63256), CbpA (Genbank Accession No.: BAA03950) and CbpB (Genbank Accession No.: NP_418813).

A “tip fragment” of a DNABII polypeptide intends a DNABII polypeptide that, using IHFalpha and IHFbeta as examples, forms the two arms of the proteins. Non-limiting examples of such include IhfA, A tip fragment: NFELRDKSSRPGRNPKTGDVV, SEQ ID NO: 1, and IhfB, B tip fragment: SLHHRQPRLGRNPKTGDSVNL, SEQ ID NO: 2.

As used herein, the term “tip-chimeric peptide” or “an IhfA5-mIhfB4_{NTHI} tip chimer” or a “tip chimer” intends a chimer of an IhfA5-mIhfB4_{NTHI} peptide that comprises, or consists essentially of, or yet further consists of: a polypeptide sequence of RPGRNPX₁TGDVVPVSARRVV-X-FSLHHRQPRLGRNPX₁TGDSV (SEQ ID NO: 3), wherein “X” is an optional amino acid linker sequence, optionally comprising, or consisting essentially of, or yet further consisting of between 1 to 20 amino acids; and wherein “X₁” is any amino acid or alternatively “X₁” is selected from the amino acids Q, R, K, S, or T. In a further aspect, “X₁” is a K or Q. In a further embodiment, the tip-chimeric peptide IhfA5-mIhfB4_{NTHI} comprises, or consists essentially of, or yet further consists of: a polypeptide sequence of RPGRNPKTGDVVPVSARRVV-X-FSLHHRQPRLGRNPKTGDSV (SEQ ID NO: 4), wherein “X” is an optional amino acid linker sequence optionally comprising, or consisting essentially of, or yet further consisting of between 1 to 20 amino acids. In certain embodiments, the linker is selected from any one or more of SEQ ID NOs: 6-8 and 26-30. In yet a further embodiment, the tip-chimeric peptide IhfA5-mIhfB4_{NTHI} comprises or consists essentially of, or yet further consists of: a polypeptide sequence of RPGRNPKTGDVVPVSARRVVGPSLFSLHHRQPRLGRNPKTGDSV (SEQ ID NO: 5).

A “tail fragment” of a DNABII polypeptide intends a region of the protein that is both exposed to the bulk medium and not occluded by DNA or other polypeptides.

As used herein, a “tail chimer” or a “tail chimeric peptide” refers to a polypeptide comprising an IHFA tail region of DNABII peptide conjugated directly or indirectly (for example via a linker) to an IHFB tail region of DNABII peptide. In yet a further embodiment, the tail region of DNABII peptide is the IhfA3-IhfB2NTHI tail chimeric peptide. In some embodiments, the tail chimer comprises an amino acid sequence as shown in SEQ ID NO: 43.

A “tip region” refers to either a “tip-chimer peptide” and/or a “tip fragment” as used herein.

An “integration host factor” or “IHF” protein is a bacterial protein that is used by bacteriophages to incorporate their DNA into the host bacteria. These are DNA binding proteins that function in genetic recombination as well as in transcription and translational regulation. They also bind extracellular microbial DNA. The genes that encode the IHF protein subunits in E. coli are himA (Genbank accession No.: POA6X7.1) and himD (POA6Y1.1) genes.

“HMGB1” is a high mobility group box (HMGB) 1 protein that is reported to bind to and distort the minor groove of DNA and is an example of an interfering agent. Recombinant or isolated protein and polypeptide are commercially available from Atgenglobal, ProSpecBio, Protein1 and Abnova. The sequences of wild type murine HMGB1 and human HMGB1 protein are as shown in SEQ ID NO: 31 and SEQ ID NO: 32, respectively.

“HU” or “histone-like protein from E. coli strain U93” refers to a class of heterodimeric proteins typically associated with E. coli. HU proteins are known to bind DNA junctions. Related proteins have been isolated from other microorganisms. The complete amino acid sequence of E. coli HU was reported by Laine et al. (1980) Eur. J. Biochem. 103 (3): 447-481. Antibodies to the HU protein are commercially available from Abcam.

A “linker” or “peptide linker” refers to a peptide sequence linked to either the N-terminus or the C-terminus of a polypeptide sequence. In one aspect, the linker is from about 1 to about 20 amino acid residues long or alternatively 2 to about 10, about 3 to about 5 amino acid residues long. Examples of peptide linkers are shown in SEQ ID NOs: 6-8 and 26-30.

As used herein, the term “DNA-binding agent” intends an agent that binds to eDNA and prevents formation of, or disrupts, extracellular structures comprising eDNA (e.g., biofilms and Neutrophil extracellular traps (NETs)).

The term “Haemophilus influenzae” refers to pathogenic bacteria that can cause many different infections such as, for example, ear infections, eye infections, and sinusitis. Many different strains of Haemophilus influenzae have been isolated and have an IhfA gene or protein. Some non-limiting examples of different strains of Haemophilus influenzae include Rd KW20, 86-028NP, R2866, PittGG, PittEE, R2846, and 2019.

“Microbial DNA” intends single or double stranded DNA from a microorganism that produces a biofilm.

As used herein, the term “label” or “detectable label” intends a directly or indirectly detectable compound or composition that is conjugated directly or indirectly to the composition to be detected, e.g., N-terminal histidine tags (N-His), magnetically active isotopes, e.g., ¹¹⁵Sn, ¹¹⁷Sn and ¹¹⁹Sn, a non-radioactive isotopes such as ¹³C and ¹⁵N, polynucleotide or protein such as an antibody so as to generate a “labeled” composition. The term also includes sequences conjugated to the polynucleotide that will provide a signal upon expression of the inserted sequences, such as green fluorescent protein (GFP) and the like. The label may be detectable by itself (e.g., radioisotope labels or fluorescent labels) or, in the case of an enzymatic label, may catalyze chemical alteration of a substrate compound or composition which is detectable. The labels can be suitable for small scale detection or more suitable for high-throughput screening. As such, suitable labels include, but are not limited to magnetically active isotopes, non-radioactive isotopes, radioisotopes, fluorochromes, chemiluminescent compounds, dyes, and proteins, including enzymes. The label may be simply detected or it may be quantified. A response that is simply detected generally comprises a response whose existence merely is confirmed, whereas a response that is quantified generally comprises a response having a quantifiable (e.g., numerically reportable) value such as an intensity, polarization, and/or other property. In luminescence or fluorescence assays, the detectable response may be generated directly using a luminophore or fluorophore associated with an assay component actually involved in binding, or indirectly using a luminophore or fluorophore associated with another (e.g., reporter or indicator) component. Examples of luminescent labels that produce signals include, but are not limited to bioluminescence and chemiluminescence. Detectable luminescence response generally comprises a change in, or an occurrence of a luminescence signal. Suitable methods and luminophores for luminescently labeling assay components are known in the art and described for example in Haugland, Richard P. (1996) Handbook of Fluorescent Probes and Research Chemicals (6^{th} ed). Examples of luminescent probes include, but are not limited to, aequorin and luciferases.

A “gene delivery vehicle” is defined as any molecule that can carry inserted polynucleotides into a host cell. Examples of gene delivery vehicles are liposomes, micelles biocompatible polymers, including natural polymers and synthetic polymers; lipoproteins; polypeptides; polysaccharides; lipopolysaccharides; artificial viral envelopes; metal particles; and bacteria, or viruses, such as baculovirus, adenovirus and retrovirus, bacteriophage, cosmid, plasmid, fungal vectors and other recombination vehicles typically used in the art which have been described for expression in a variety of eukaryotic and prokaryotic hosts, and may be used for gene therapy as well as for simple protein expression.

A polynucleotide of this disclosure can be delivered to a cell or tissue using a gene delivery vehicle. “Gene delivery,” “gene transfer,” “transducing,” and the like as used herein, are terms referring to the introduction of an exogenous polynucleotide (sometimes referred to as a “transgene”) into a host cell, irrespective of the method used for the introduction. Such methods include a variety of well-known techniques such as vector-mediated gene transfer (by, e.g., viral infection/transfection, or various other protein-based or lipid-based gene delivery complexes) as well as techniques facilitating the delivery of “naked” polynucleotides (such as electroporation, “gene gun” delivery and various other techniques used for the introduction of polynucleotides). The introduced polynucleotide may be stably or transiently maintained in the host cell. Stable maintenance typically requires that the introduced polynucleotide either contains an origin of replication compatible with the host cell or integrates into a replicon of the host cell such as an extrachromosomal replicon (e.g., a plasmid) or a nuclear or mitochondrial chromosome. A number of vectors are known to be capable of mediating transfer of genes to mammalian cells, as is known in the art and described herein.

As used herein the term “eDNA” refers to extracellular DNA found as a component to pathogenic biofilms.

As used herein, the ESKAPE pathogens include Enterococcus faecium, Staphylococcus aureus, Klebsiella pneumoniae, Acinetobacter baumannii, Pseudomonas aeruginosa, and Enterobacter species. These pathogens are the leading cause of nosocomial infections throughout the world.

A “plasmid” is an extra-chromosomal DNA molecule separate from the chromosomal DNA which is capable of replicating independently of the chromosomal DNA. In many cases, it is circular and double-stranded. Plasmids provide a mechanism for horizontal gene transfer within a population of microbes and typically provide a selective advantage under a given environmental state. Plasmids may carry genes that provide resistance to naturally occurring antibiotics in a competitive environmental niche, or alternatively the proteins produced may act as toxins under similar circumstances.

“Plasmids” used in genetic engineering are called “plasmid vectors”. Many plasmids are commercially available for such uses. The gene to be replicated is inserted into copies of a plasmid containing genes that make cells resistant to particular antibiotics and a multiple cloning site (MCS, or polylinker), which is a short region containing several commonly used restriction sites allowing the easy insertion of DNA fragments at this location. Another major use of plasmids is to make large amounts of proteins. In this case, researchers grow bacteria containing a plasmid harboring the gene of interest. Just as the bacterium produces proteins to confer its antibiotic resistance, it can also be induced to produce large amounts of proteins from the inserted gene. This is a cheap and easy way of mass-producing a gene or the protein it then codes for.

A “yeast artificial chromosome” or “YAC” refers to a vector used to clone large DNA fragments (larger than 100 kb and up to 3000 kb). It is an artificially constructed chromosome and contains the telomeric, centromeric, and replication origin sequences needed for replication and preservation in yeast cells. Built using an initial circular plasmid, they are linearized by using restriction enzymes, and then DNA ligase can add a sequence or gene of interest within the linear molecule by the use of cohesive ends. Yeast expression vectors, such as YACs, YIps (yeast integrating plasmid), and YEps (yeast episomal plasmid), are extremely useful as one can get eukaryotic protein products with posttranslational modifications as yeasts are themselves eukaryotic cells, however YACs have been found to be more unstable than BACs, producing chimeric effects.

A “viral vector” is defined as a recombinantly produced virus or viral particle that comprises a polynucleotide to be delivered into a host cell, either, ex vivo or. Examples of viral vectors include retroviral vectors, adenovirus vectors, adeno-associated virus vectors, alphavirus vectors and the like. Infectious tobacco mosaic virus (TMV)-based vectors can be used to manufacturer proteins and have been reported to express Griffithsin in tobacco leaves (O'Keefe et al. (2009) Proc. Nat. Acad. Sci. USA 106(15):6099-6104). Alphavirus vectors, such as Semliki Forest virus-based vectors and Sindbis virus-based vectors, have also been developed for use in gene therapy and immunotherapy. See, Schlesinger & Dubensky (1999) Curr. Opin. Biotechnol. 5:434-439 and Ying et al. (1999) Nat. Med. 5(7):823-827. In aspects where gene transfer is mediated by a retroviral vector, a vector construct refers to the polynucleotide comprising the retroviral genome or part thereof, and a therapeutic gene.

As used herein, “retroviral mediated gene transfer” or “retroviral transduction” carries the same meaning and refers to the process by which a gene or nucleic acid sequences are stably transferred into the host cell by virtue of the virus entering the cell and integrating its genome into the host cell genome. The virus can enter the host cell via its normal mechanism of infection or be modified such that it binds to a different host cell surface receptor or ligand to enter the cell. As used herein, retroviral vector refers to a viral particle capable of introducing exogenous nucleic acid into a cell through a viral or viral-like entry mechanism.

Retroviruses carry their genetic information in the form of RNA; however, once the virus infects a cell, the RNA is reverse transcribed into the DNA form which integrates into the genomic DNA of the infected cell. The integrated DNA form is called a provirus.

In aspects where gene transfer is mediated by a DNA viral vector, such as an adenovirus (Ad) or adeno-associated virus (AAV), a vector construct refers to the polynucleotide comprising the viral genome or part thereof, and a transgene. Adenoviruses (Ads) are a relatively well characterized, homogenous group of viruses, including over 50 serotypes. See, e.g., International PCT Application No. WO 95/27071. Ads do not require integration into the host cell genome. Recombinant Ad derived vectors, particularly those that reduce the potential for recombination and generation of wild-type virus, have also been constructed. See, International PCT Application Nos. WO 95/00655 and WO 95/11984. Wild-type AAV has high infectivity and specificity integrating into the host cell's genome. See, Hermonat & Muzyczka (1984) Proc. Natl. Acad. Sci. USA 81:6466-6470 and Lebkowski et al. (1988) Mol. Cell. Biol. 8:3988-3996.

Vectors that contain both a promoter and a cloning site into which a polynucleotide can be operatively linked are known in the art. Such vectors are capable of transcribing RNA or, and are commercially available from sources such as Stratagene (La Jolla, CA) and Promega Biotech (Madison, WI). In order to optimize expression and/or transcription, it may be necessary to remove, add or alter 5’ and/or 3′ untranslated portions of the clones to eliminate extra, potential inappropriate alternative translation initiation codons or other sequences that may interfere with or reduce expression, either at the level of transcription or translation. Alternatively, consensus ribosome binding sites can be inserted immediately 5′ of the start codon to enhance expression.

Gene delivery vehicles also include DNA/liposome complexes, micelles and targeted viral protein-DNA complexes. Liposomes that also comprise a targeting antibody or fragment thereof can be used in the methods of this disclosure. In addition to the delivery of polynucleotides to a cell or cell population, direct introduction of the proteins described herein to the cell or cell population can be done by the non-limiting technique of protein transfection, alternatively culturing conditions that can enhance the expression and/or promote the activity of the proteins of this disclosure are other non-limiting techniques.

“Inhibiting, preventing or breaking down” a biofilm intends the prophylactic or therapeutic reduction in the structure of a biofilm. In one aspect, the definition excludes prevention of a biofilm. In another aspect, the terms “inhibiting, competing or titrating” intend a reduction in the formation of the DNA/protein matrix that is a component of a microbial biofilm.

A “bent polynucleotide” intends a double strand polynucleotide that contains a small loop on one strand which does not pair with the other strand and any polynucleotide where the end to end distance is reduced beyond natural thermal fluctuations i.e. that is bending beyond the persistence length of 150 bp for native B-form double stranded DNA. In some embodiments, the loop is from 1 base to about 20 bases long, or alternatively from 2 bases to about 15 bases long, or alternatively from about 3 bases to about 12 bases long, or alternatively from about 4 bases to about 10 bases long, or alternatively has about 4, 5, or 6, or 7, or 8, or 9 or 10 bases.

A “subject” of diagnosis or treatment is a cell or an animal such as a mammal or a human. Non-human animals subject to diagnosis or treatment and are those subject to infections or animal models, for example, simians, murines, such as, rats, mice, chinchilla, canine, such as dogs, leporids, such as rabbits, livestock, sport animals and pets.

The term “protein”, “peptide” and “polypeptide” are used interchangeably and in their broadest sense refer to a compound of two or more subunit amino acids, amino acid analogs or peptidomimetics. The subunits may be linked by peptide bonds. In another embodiment, the subunit may be linked by other bonds, e.g., ester, ether, etc. A protein or peptide must contain at least two amino acids and no limitation is placed on the maximum number of amino acids which may comprise a protein's or peptide's sequence. As used herein the term “amino acid” refers to either natural and/or unnatural or synthetic amino acids, including glycine and both the D and L optical isomers, amino acid analogs and peptidomimetics.

The term “isolated” or “recombinant” as used herein with respect to nucleic acids, such as DNA or RNA, refers to molecules separated from other DNAs or RNAs, respectively that are present in the natural source of the macromolecule as well as polypeptides. The term “isolated or recombinant nucleic acid” is meant to include nucleic acid fragments which are not naturally occurring as fragments and would not be found in the natural state. The term “isolated” is also used herein to refer to polynucleotides, polypeptides and proteins that are isolated from other cellular proteins and is meant to encompass both purified and recombinant polypeptides. In other embodiments, the term “isolated or recombinant” means separated from constituents, cellular and otherwise, in which the cell, tissue, polynucleotide, peptide, polypeptide, protein, antibody or fragment(s) thereof, which are normally associated in nature. For example, an isolated cell is a cell that is separated from tissue or cells of dissimilar phenotype or genotype. An isolated polynucleotide is separated from the 3′ and 5′ contiguous nucleotides with which it is normally associated in its native or natural environment, e.g., on the chromosome. As is apparent to those of skill in the art, a non-naturally occurring polynucleotide, peptide, polypeptide, protein, antibody, or fragment(s) thereof, does not require “isolation” to distinguish it from its naturally occurring counterpart.

It is to be inferred without explicit recitation and unless otherwise intended, that when the present disclosure relates to a polypeptide, protein, polynucleotide, or antibody, an equivalent or a biologically equivalent of such is intended within the scope of this disclosure. As used herein, the term “biological equivalent thereof” is intended to be synonymous with “equivalent thereof” when referring to a reference protein, antibody, polypeptide or nucleic acid, intends those having minimal homology while still maintaining desired structure or functionality. Unless specifically recited herein, it is contemplated that any polynucleotide, polypeptide or protein mentioned herein also includes equivalents thereof. For example, an equivalent intends at least about 70% homology or identity, or alternatively about 80% homology or identity and alternatively, at least about 85%, or alternatively at least about 90%, or alternatively at least about 95% or alternatively 98% percent homology or identity and exhibits substantially equivalent biological activity to the reference protein, polypeptide or nucleic acid. In another aspect, the term intends a polynucleotide that hybridizes under conditions of high stringency to the reference polynucleotide or its complement.

A polynucleotide or polynucleotide region (or a polypeptide or polypeptide region) having a certain percentage (for example, 80%, 85%, 90% or 95%) of “sequence identity” to another sequence means that, when aligned, that percentage of bases (or amino acids) are the same in comparing the two sequences. The alignment and the percent homology or sequence identity can be determined using software programs known in the art, for example those described in Current Protocols in Molecular Biology (Ausubel et al., eds. 1987) Supplement 30, section 7.7.18, Table 7.7.1. Preferably, default parameters are used for alignment. A preferred alignment program is BLAST, using default parameters. In particular, preferred programs are BLASTN and BLASTP, using the following default parameters: Genetic code=standard; filter=none; strand=both; cutoff=60; expect=10; Matrix=BLOSUM62; Descriptions=50 sequences; sort by=HIGH SCORE; Databases=non-redundant, GenBank+EMBL+DDBJ+PDB+GenBank CDS translations+SwissProtein+SPupdate+PIR. Details of these programs can be found at the following Internet address: ncbi.nlm.nih.gov/cgi-bin/BLAST.

“Homology” or “identity” or “similarity” refers to sequence similarity between two peptides or between two nucleic acid molecules. Homology can be determined by comparing a position in each sequence which may be aligned for purposes of comparison. When a position in the compared sequence is occupied by the same base or amino acid, then the molecules are homologous at that position. A degree of homology between sequences is a function of the number of matching or homologous positions shared by the sequences. An “unrelated” or “non-homologous” sequence shares less than 30% identity or alternatively less than 25% identity, less than 20% identity, or alternatively less than 10% identity with one of the sequences of the present disclosure.

“Homology” or “identity” or “similarity” can also refer to two nucleic acid molecules that hybridize under stringent conditions to the reference polynucleotide or its complement.

“Hybridization” refers to a reaction in which one or more polynucleotides react to form a complex that is stabilized via hydrogen bonding between the bases of the nucleotide residues. The hydrogen bonding may occur by Watson-Crick base pairing, Hoogstein binding, or in any other sequence-specific manner. The complex may comprise two strands forming a duplex structure, three or more strands forming a multi-stranded complex, a single self-hybridizing strand, or any combination of these. A hybridization reaction may constitute a step in a more extensive process, such as the initiation of a PCR reaction, or the enzymatic cleavage of a polynucleotide by a ribozyme.

Examples of stringent hybridization conditions include: incubation temperatures of about 25° C. to about 37° C.; hybridization buffer concentrations of about 6×SSC to about 10×SSC; formamide concentrations of about 0% to about 25%; and wash solutions from about 4×SSC to about 8×SSC. Examples of moderate hybridization conditions include: incubation temperatures of about 40° C. to about 50° C.; buffer concentrations of about 9×SSC to about 2×SSC; formamide concentrations of about 30% to about 50%; and wash solutions of about 5×SSC to about 2×SSC. Examples of high stringency conditions include: incubation temperatures of about 55° C. to about 68° C.; buffer concentrations of about 1×SSC to about 0.1×SSC; formamide concentrations of about 55% to about 75%; and wash solutions of about 1×SSC, 0.1×SSC, or deionized water. In general, hybridization incubation times are from 5 minutes to 24 hours, with 1, 2, or more washing steps, and wash incubation times are about 1, 2, or 15 minutes. SSC is 0.15 M NaCl and 15 mM citrate buffer. It is understood that equivalents of SSC using other buffer systems can be employed.

A “subject” of diagnosis or treatment is a cell or an animal such as a mammal, or a human. Non-human animals subject to diagnosis or treatment and are those subject to infections or animal models, for example, simians, murines, such as, rats, mice, chinchilla, canine, such as dogs, leporids, such as rabbits, livestock, sport animals, and pets. The term “subject,” “host,” “individual,” and “patient” are as used interchangeably herein to refer to animals, typically mammalian animals. Non-limiting examples of mammals include humans, non-human primates (e.g., apes, gibbons, chimpanzees, orangutans, monkeys, macaques, and the like), domestic animals (e.g., dogs and cats), farm animals (e.g., horses, cows, goats, sheep, pigs) and experimental animals (e.g., mouse, rat, rabbit, guinea pig). In some embodiments a mammal is a human. A mammal can be any age or at any stage of development (e.g., an adult, teen, child, infant, or a mammal in utero). A mammal can be male or female. In some embodiments a subject is a human.

A host cell intends a eukaryotic or prokaryotic cell comprising an exogenous agent. “Eukaryotic cells” comprise all of the life kingdoms except Monera. They can be easily distinguished through a membrane-bound nucleus. Animals, plants, fungi, and protists are eukaryotes or organisms whose cells are organized into complex structures by internal membranes and a cytoskeleton. The most characteristic membrane-bound structure is the nucleus. Unless specifically recited, the term “host” includes a eukaryotic host, including, for example, yeast, higher plant, insect and mammalian cells. Non-limiting examples of eukaryotic cells or hosts include simian, bovine, porcine, murine, rat, avian, reptilian and human.

“Prokaryotic cells” that usually lack a nucleus or any other membrane-bound organelles and are divided into two domains, bacteria and archaea. In addition to chromosomal DNA, these cells can also contain genetic information in a circular loop called on episome. Bacterial cells are very small, roughly the size of an animal mitochondrion (about 1-2 μm in diameter and 10 μm long). Prokaryotic cells feature three major shapes: rod shaped, spherical, and spiral. Instead of going through elaborate replication processes like eukaryotes, bacterial cells divide by binary fission. Examples include but are not limited to Bacillus bacteria, E. coli bacterium, and Salmonella bacterium.

As used herein, the terms “treating,” “treatment” and the like are used herein to mean obtaining a desired pharmacologic and/or physiologic effect. The effect may be prophylactic in terms of completely or partially preventing a disorder or sign or symptom thereof and/or may be therapeutic in terms of a partial or complete cure for a disorder and/or adverse effect attributable to the disorder. In one aspect, “treatment” excludes prevention.

To “prevent” intends to prevent a disorder or effect or in a system or subject that is predisposed to the disorder or effect. An example of such is preventing the formation of a biofilm in a system that is infected with a microorganism known to produce one.

“Pharmaceutically acceptable carriers” refers to any diluents, excipients or carriers that may be used in the compositions of the disclosure. Pharmaceutically acceptable carriers include ion exchangers, alumina, aluminum stearate, lecithin, serum proteins, such as human serum albumin, buffer substances, such as phosphates, glycine, sorbic acid, potassium sorbate, partial glyceride mixtures of saturated vegetable fatty acids, water, salts or electrolytes, such as protamine sulfate, disodium hydrogen phosphate, potassium hydrogen phosphate, sodium chloride, zinc salts, colloidal silica, magnesium trisilicate, polyvinyl pyrrolidone, cellulose-based substances, polyethylene glycol, sodium carboxymethylcellulose, polyacrylates, waxes, polyethylene-polyoxypropylene-block polymers, polyethylene glycol and wool fat. Suitable pharmaceutical carriers are described in Remington's Pharmaceutical Sciences, Mack Publishing Company, a standard reference text in this field. They are preferably selected with respect to the intended form of administration, that is, oral tablets, capsules, elixirs, syrups and the like and consistent with conventional pharmaceutical practices.

“MIC” is the acronym for minimum inhibitory and defines in vitro levels of susceptibility or resistance of specific bacterial strains to applied antibiotic. Methods to convert MIC to a therapeutic amount are known in the art and describe in Kowalska-Krochmal and Wicher, Pathogens (2021) February; 10 (2): 165.

“Administration” can be affected in one dose, continuously or intermittently throughout the course of treatment. Methods of determining the most effective means and dosage of administration are known to those of skill in the art and will vary with the composition used for therapy, the purpose of the therapy, the target cell being treated and the subject being treated. Single or multiple administrations can be carried out with the dose level and pattern being selected by the treating physician. Suitable dosage formulations and methods of administering the agents are known in the art. Route of administration can also be determined and method of determining the most effective route of administration are known to those of skill in the art and will vary with the composition used for treatment, the purpose of the treatment, the health condition or disease stage of the subject being treated and target cell or tissue. Non-limiting examples of route of administration include oral administration, nasal administration, injection and topical application.

The term “encode” as it is applied to polynucleotides refers to a polynucleotide which is said to “encode” a polypeptide if, in its native state or when manipulated by methods well known to those skilled in the art, it can be transcribed and/or translated to produce the mRNA for the polypeptide and/or a fragment thereof. The antisense strand is the complement of such a nucleic acid, and the encoding sequence can be deduced therefrom.

The term “effective amount” refers to a quantity sufficient to achieve a beneficial or desired result or effect. In the context of therapeutic or prophylactic applications, the effective amount will depend on the type and severity of the condition at issue and the characteristics of the individual subject, such as general health, age, sex, body weight, and tolerance to pharmaceutical compositions. In the context of an immunogenic composition, in some embodiments the effective amount is the amount sufficient to result in a protective response against a pathogen. In other embodiments, the effective amount of an immunogenic composition is the amount sufficient to result in antibody generation against the antigen. In some embodiments, the effective amount is the amount required to confer passive immunity on a subject in need thereof. With respect to immunogenic compositions, in some embodiments the effective amount will depend on the intended use, the degree of immunogenicity of a particular antigenic compound, and the health/responsiveness of the subject's immune system, in addition to the factors described above. The skilled artisan will be able to determine appropriate amounts depending on these and other factors.

In the case of an application, in some embodiments the effective amount will depend on the size and nature of the application in question. It will also depend on the nature and sensitivity of the target and the methods in use. The skilled artisan will be able to determine the effective amount based on these and other considerations. The effective amount may comprise one or more administrations of a composition depending on the embodiment.

The agents and compositions can be used in the manufacture of medicaments and for the treatment of humans and other animals by administration in accordance with conventional procedures, such as an active ingredient in pharmaceutical compositions.

An agent of the present disclosure can be administered for therapy by any suitable route of administration. It will also be appreciated that the preferred route will vary with the condition and age of the recipient and the disease being treated.

An example of a solid phase support include glass, polystyrene, polypropylene, polyethylene, dextran, nylon, amylases, natural and modified celluloses, polyacrylamides, gabbros and magnetite. The nature of the carrier can be either soluble to some extent or insoluble. The support material may have virtually any possible structural configuration so long as the coupled molecule is capable of binding to a polynucleotide, polypeptide or antibody. Thus, the support configuration may be spherical, as in a bead or cylindrical, as in the inside surface of a test tube or the external surface of a rod. Alternatively, the surface may be flat such as a sheet, test strip, etc, or alternatively polystyrene beads. Those skilled in the art will know many other suitable carriers for binding antibody or antigen or will be able to ascertain the same by use of routine experimentation.

As used herein, an “antibody” includes whole antibodies and any antigen-binding fragment or a single chain thereof. Thus the term “antibody” includes any protein or peptide containing molecule that comprises at least a portion of an immunoglobulin molecule. Examples of such include, but are not limited to a complementarity determining region (CDR) of a heavy or light chain or a ligand binding portion thereof, a heavy chain or light chain variable region, a heavy chain or light chain constant region, a framework (FR) region or any portion thereof or at least one portion of a binding protein. The antibodies can be polyclonal or monoclonal and can be isolated from any suitable biological source, e.g., murine, rat, sheep or canine.

The terms “antibody,” “antibodies” and “immunoglobulin” also include immunoglobulins of any isotype, fragments of antibodies which retain specific binding to antigen, including, but not limited to, Fab, Fab′, F (ab) 2, Fv, scFv, dsFv, Fd fragments, dAb, VH, VL, VhH, and V-NAR domains; minibodies, diabodies, triabodies, tetrabodies and kappa bodies; and multispecific antibody fragments formed from antibody fragments. Examples of such include, but are not limited to a complementarity determining region (CDR) of a heavy or light chain or a ligand binding portion thereof, a heavy chain or light chain variable region (which is also referred to herein as a variable domain), a heavy chain or light chain constant region (which is also referred to herein as a constant domain), a framework (FR) region, or any portion thereof, at least one portion of a binding protein, chimeric antibodies, humanized antibodies, single-chain antibodies, and fusion proteins comprising an antigen-binding portion of an antibody and a non-antibody protein. The variable regions of the heavy and light chains of the immunoglobulin molecule contain a binding domain that interacts with an antigen. The constant regions of the antibodies (Abs) may mediate the binding of the immunoglobulin to host tissues. The term “anti-” when used before a protein name, anti-DNABII, anti-IHF, anti-HU, anti-tip chimer, for example, refers to a monoclonal or polyclonal antibody that binds and/or has an affinity to a particular protein. For example, “anti-IHF” refers to an antibody that binds to the IHF protein. The specific antibody may have affinity or bind to proteins other than the protein it was raised against. For example, anti-IHF, while specifically raised against the IHF protein, may also bind other proteins that are related either through sequence homology or through structure homology.

Complementarity determining regions (CDRs) are part of the variable region of an antibody or a T cell receptor generated by B-cell s and T-cells respectively, wherein these molecules bind to their specific antigen (also called epitope). In certain embodiments, the terms “variable region” and “variable domain” are used interchangeably, referring to the polypeptide of a light or heavy chain of an antibody that varies greatly in its sequence of amino acid residues from one antibody to another, and that determines the conformation of the combining site which confers the specificity of the antibody for a particular antigen. In a further embodiment, the variable region is about 90 amino acids long to about 200 amino acids long, including but not limited to about 100 amino acids long, or alternatively about 110 amino acids long, or alternatively about 120 amino acids long, or alternatively about 130 amino acids long, or alternatively about 140 amino acids long, or alternatively about 150 amino acids long, or alternatively about 160 amino acids long, or alternatively about 170 amino acids long, or alternatively about 180 amino acids long, or alternatively about 190 amino acids long. In certain embodiments, a variable region of an amino acid sequence, as used herein, refers to that the first about 100 amino acids, or alternatively about 110 amino acids, or alternatively about 120 amino acids, or alternatively about 130 amino acids, or alternatively about 140 amino acids, or alternatively about 150 amino acids of the amino acid sequence (including or excluding a signal peptide if applicable) is the variable region.

A set of CDRs constitutes a paratope also called an antigen-binding site, which is a part of an antibody that recognizes and binds to an antigen. There are three CDRs (CDR1, CDR2 and CDR3), arranged non-consecutively, optionally from the amino terminus to the carboxyl terminus, on the amino acid sequence of a variable region of an antigen receptor, such as a heavy chain or a light chain. As used herein, CDRn refers to a CDRn in an immunoglobulin chain or derived from an immunoglobulin chain, wherein the number n is selected from 1-3. In one embodiment, CDRLn refers to a CDRn in a light chain or derived from a light chain, wherein the number n is selected from 1-3; while CDRHn refers to a CDRn in a heavy chain or derived from a heavy chain, wherein the number n is selected from 1-3. In certain embodiments, framework region (FR) refers to the part of a variable region which is not a CDR. In certain embodiments, FRn refers to a FR in a heavy chain or a light chain or derived from a heavy chain or a light chain, and wherein the number n is selected from 1-4. In certain embodiments, a variable region comprises or consists essentially of, or yet further consists of the following (optionally following the order as provided, and further optionally from the amino terminus to the carboxyl terminus): FR1, CDR1, FR2, CDR2, FR3, CDR3 and FR4.

Variable regions and/or CDRs of an antibody or a fragment thereof can be determined by one of skill in the art, for example, using publicly or commercially available tools. Non-limiting examples of such tools include, IgBlast (accessible at www.ncbi.nlm.nih.gov/igblast/), Scaligner (available from drugdesigntech at www.scaligner.com/), IMGT rules and/or tools (see, for example, www.imgt.org/IMGTScientificChart/Nomenclature/IMGT-FRCDRdefinition.html, also accessible at www.imgt.org/), Chothia Canonical Assignment (accessible at www.bioinf.org.uk/abs/chothia.html), Antigen receptor Numbering And Receptor Classification (ANARCI, accessible at opig.stats.ox.ac.uk/webapps/newsabdab/sabpred/anarci/), the Kabat numbering method/scheme (e.g., Kabat, E. A., et al., (1991) Sequences of Proteins of Immunological Interest, Fifth Edition, U.S. Department of Health and Human Services, NIH Publication No. 91-3242,) or the Paratome web server (accessible at www.ofranlab.org/paratome/, see Vered Kunik, et al, Nucleic Acids Research, Volume 40, Issue W1, 1 Jul. 2012, Pages W521-W524).

The antibodies can be polyclonal, monoclonal, multispecific (e.g., bispecific antibodies), and antibody fragments, so long as they exhibit the desired biological activity. Antibodies can be isolated from any suitable biological source, e.g., murine, rat, sheep and canine.

The terms “polyclonal antibody” or “polyclonal antibody composition” as used herein refer to a preparation of antibodies that are derived from different B-cell lines. They are a mixture of immunoglobulin molecules secreted against a specific antigen, each recognizing a different epitope.

As used herein, “monoclonal antibody” refers to an antibody obtained from a substantially homogeneous antibody population. Monoclonal antibodies are highly specific, as each monoclonal antibody is directed against a single determinant on the antigen. The antibodies may be detectably labeled, e.g., with a radioisotope, an enzyme which generates a detectable product, a fluorescent protein, and the like. The antibodies may be further conjugated to other moieties, such as members of specific binding pairs, e.g., biotin (member of biotin-avidin specific binding pair), and the like. The antibodies may also be bound to a solid support, including, but not limited to, polystyrene plates or beads, and the like.

Monoclonal antibodies may be generated using hybridoma techniques or recombinant DNA methods known in the art. A hybridoma is a cell that is produced in the laboratory from the fusion of an antibody-producing lymphocyte and a non-antibody producing cancer cell, usually a myeloma or lymphoma. A hybridoma proliferates and produces a continuous sample of a specific monoclonal antibody. Alternative techniques for generating or selecting antibodies include exposure of lymphocytes to antigens of interest, and screening of antibody display libraries in cells, phage, or similar systems.

The term “human antibody” as used herein, is intended to include antibodies having variable and constant regions derived from human germline immunoglobulin sequences. The human antibodies disclosed herein may include amino acid residues not encoded by human germline immunoglobulin sequences (e.g., mutations introduced by random or site-specific mutagenesis or by somatic mutation). However, the term “human antibody” as used herein, is not intended to include antibodies in which CDR sequences derived from the germline of another mammalian species, such as a mouse, have been grafted onto human framework sequences. Thus, as used herein, the term “human antibody” refers to an antibody in which substantially every part of the protein (e.g., CDR, framework, CL, CH domains (e.g., C_{H1}, C_{H2}, C_{H3}), hinge, (VL, VH)) is substantially non-immunogenic in humans, with only minor sequence changes or variations. Similarly, antibodies designated primate (monkey, baboon, chimpanzee, etc.), rodent (mouse, rat, rabbit, guinea pig, hamster, and the like) and other mammals designate such species, sub-genus, genus, sub-family, family specific antibodies. Further, chimeric antibodies include any combination of the above. Such changes or variations optionally retain or reduce the immunogenicity in humans or other species relative to non-modified antibodies. Thus, a human antibody is distinct from a chimeric or humanized antibody. It is pointed out that a human antibody can be produced by a non-human animal or prokaryotic or eukaryotic cell that is capable of expressing functionally rearranged human immunoglobulin (e.g., heavy chain and/or light chain) genes. Further, when a human antibody is a single chain antibody, it can comprise a linker peptide that is not found in native human antibodies. For example, an Fv can comprise a linker peptide, such as two to about eight glycine or other amino acid residues, which connects the variable region of the heavy chain and the variable region of the light chain. Such linker peptides are considered to be of human origin.

As used herein, a human antibody is “derived from” a particular germline sequence if the antibody is obtained from a system using human immunoglobulin sequences, e.g., by immunizing a transgenic mouse carrying human immunoglobulin genes or by screening a human immunoglobulin gene library. A human antibody that is “derived from” a human germline immunoglobulin sequence can be identified as such by comparing the amino acid sequence of the human antibody to the amino acid sequence of human germline immunoglobulins. A selected human antibody typically is at least 90% identical in amino acids sequence to an amino acid sequence encoded by a human germline immunoglobulin gene and contains amino acid residues that identify the human antibody as being human when compared to the germline immunoglobulin amino acid sequences of other species (e.g., murine germline sequences). In certain cases, a human antibody may be at least 95%, or even at least 96%, 97%, 98%, or 99% identical in amino acid sequence to the amino acid sequence encoded by the germline immunoglobulin gene. Typically, a human antibody derived from a particular human germline sequence will display no more than 10 amino acid differences from the amino acid sequence encoded by the human germline immunoglobulin gene. In certain cases, the human antibody may display no more than 5, or even no more than 4, 3, 2, or 1 amino acid difference from the amino acid sequence encoded by the germline immunoglobulin gene.

As used herein, the term “humanized antibody” or “humanized immunoglobulin” refers to a human/non-human chimeric antibody that contains a minimal sequence derived from non-human immunoglobulin. For the most part, humanized antibodies are human immunoglobulins (recipient antibody) in which residues from a variable region or a fragment thereof (for example, 1, 2, 3, 4, 5, or all 6 CDRs) of the recipient are replaced by residues from a variable region or a fragment thereof (for example, 1, 2, 3, 4, 5, or all 6 CDRs) of a non-human species (donor antibody) such as mouse, rat, rabbit, or non-human primate having the desired specificity, affinity and capacity. Humanized antibodies may comprise residues that are not found in the recipient antibody or in the donor antibody. The humanized antibody can optionally also comprise at least a portion of an immunoglobulin constant region (Fc), typically that of a human immunoglobulin, a non-human antibody containing one or more amino acids in a framework region, a constant region or a CDR, that have been substituted with a correspondingly positioned amino acid from a human antibody. Without wishing to be bound by the theory, humanized antibodies produce a reduced immune response in a human host, as compared to a non-humanized version of the same antibody. The humanized antibodies may have conservative amino acid substitutions which have substantially no effect on antigen-binding or other antibody functions. Conservative substitutions groupings include: glycine-alanine, valine-leucine-isoleucine, phenylalanine-tyrosine, lysine-arginine, alanine-valine, serine-threonine and asparagine-glutamine. Specifically, the humanized antibodies as disclosed herein specifically binds to a DNABII polypeptide or a fragment thereof (such as the tip chimeric peptide or the tail chimeric peptide) with certain range(s) of one or more of the following: EC₅₀, Kₒₙ, K_{off}, K_{A} and/or K_{D}, and inhibits or releases certain cytokine(s) upon treating a subject. In a further embodiment, the humanized antibody specifically binding to the tip region of a DNABII polypeptide (such as the tip chimeric peptide) disrupts biofilm both and. In addition, the process of humanization, while a rational design process, may produce unexpected changes (positive or negative) in, e.g., binding affinity, antigen specificity, or physical properties such as solubility or aggregability; hence, properties of humanized antibodies are not inherently predictable from the properties of the starting non-human antibody.

In one embodiment, an antibody as used herein may be a recombinant antibody. The term “recombinant antibody”, as used herein, includes all antibodies that are prepared, expressed, created or isolated by recombinant means, such as antibodies isolated from an animal (e.g., a mouse) that is transgenic or transchromosomal for immunoglobulin genes or a hybridoma prepared therefrom, antibodies isolated from a host cell transformed to express the antibody, e.g., from a transfectoma, antibodies isolated from a recombinant, combinatorial antibody library, and antibodies prepared, expressed, created or isolated by any other means that involve splicing of immunoglobulin (Ig) gene sequences to other DNA sequences. In certain embodiments, however, such recombinant antibodies can be subjected to mutagenesis (or, when an animal transgenic for Ig sequences is used, somatic mutagenesis) and thus the amino acid sequences of the VH and VL regions of the recombinant antibodies are sequences that may not naturally exist within the antibody germline repertoire. Methods to making these antibodies are known in the art.

In one embodiment, an antibody as used herein may be a chimeric antibody. As used herein, chimeric antibodies are antibodies whose light and heavy chain genes have been constructed, typically by genetic engineering, from antibody variable and constant region genes belonging to different species.

In addition, the antibodies disclosed herein may be engineered to include modifications within the Fc region to alter one or more functional properties of the antibody, such as serum half-fife, complement fixation, Fc receptor binding, and/or antigen-dependent cellular cytotoxicity. Such modifications include, but are not limited to, alterations of the number of cysteine residues in the hinge region to facilitate assembly of the light and heavy chains or to increase or decrease the stability of the antibody (U.S. Pat. No. 5,677,425) and amino acid mutations in the Fc hinge region to decrease the biological half-life of the antibody (U.S. Pat. No. 6,165,745).

Additionally, the antibodies disclosed herein may be chemically modified. Glycosylation of an antibody can be altered, for example, by modifying one or more sites of glycosylation within the antibody sequence to increase the affinity of the antibody for antigen (U.S. Pat. Nos. 5,714,350 and 6,350,861). Alternatively, to increase antibody-dependent cell-mediated cytotoxicity, a hypofucosylated antibody having reduced amounts of fucosyl residues or an antibody having increased bisecting GlcNac structures can be obtained by expressing the antibody in a host cell with altered glycosylation mechanism (Shields, R. L. et al. (2002) J. Biol. Chem. 277:26733-26740; Umana et al. (1999) Nat. Biotech. 17:176-180).

The antibodies disclosed herein can be pegylated to increase biological half-life by reacting the antibody or antigen-binding fragment thereof with polyethylene glycol (PEG) or a reactive ester or aldehyde derivative of PEG, under conditions in which one or more PEG groups become attached to the antibody or antibody fragment. Antibody pegylation may be carried out by an acylation reaction or an alkylation reaction with a reactive PEG molecule (or an analogous reactive water-soluble polymer). As used herein, the term “polyethylene glycol” is intended to encompass any of the forms of PEG that have been used to derivatize other proteins, such as mono (C1-C10) alkoxy- or aryloxy-polyethylene glycol or polyethylene glycol-maleimide. The antibody to be pegylated can be an aglycosylated antibody. Methods for pegylating proteins are known in the art and can be applied to the antibodies disclosed herein (EP 0154316 and EP 0401384).

Additionally, antibodies may be chemically modified by conjugating or fusing the antigen-binding region of the antibody to serum protein, such as human serum albumin, to increase half-life of the resulting molecule. Such approach is for example described in EP 0322094 and EP 0486525.

The antibodies or fragments thereof of the present disclosure may be conjugated to a diagnostic agent and used diagnostically, for example, to monitor the development or progression of a disease and determine the efficacy of a given treatment regimen. Examples of diagnostic agents include enzymes, prosthetic groups, fluorescent materials, luminescent materials, bioluminescent materials, radioactive materials, positron emitting metals using various positron emission tomographies, and nonradioactive paramagnetic metal ions. The detectable substance may be coupled or conjugated either directly to the antibody or fragment thereof, or indirectly, through a linker using techniques known in the art. Examples of suitable enzymes include horseradish peroxidase, alkaline phosphatase, beta-galactosidase, or acetylcholinesterase. Examples of suitable prosthetic group complexes include streptavidin/biotin and avidin/biotin. Examples of suitable fluorescent materials include umbelliferone, fluorescein, fluorescein isothiocyanate, rhodamine, dichlorotriazinylamine fluorescein, dansyl chloride or phycoerythrin. An example of a luminescent material includes luminol. Examples of bioluminescent materials include luciferase, luciferin, and aequorin. Examples of suitable radioactive material include 1251, 1311, Indium-111, Lutetium-171, Bismuth-212, Bismuth-213, Astatine-211, Copper-62, Copper-64, Copper-67, Yttrium-90, Iodine-125, Iodine-131, Phosphorus-32, Phosphorus-33, Scandium-47, Silver-111, Gallium-67, Praseodymium-142, Samarium-153, Terbium-161, Dysprosium-166, Holmium-166, Rhenium-186, Rhenium-188, Rhenium-189, Lead-212, Radium-223, Actinium-225, Iron-59, Selenium-75, Arsenic-77, Strontium-89, Molybdenum-99, Rhodium-1105, Palladium-109, Praseodymium-143, Promethium-149, Erbium-169, Iridium-194, Gold-198, Gold-199, and Lead-211. Monoclonal antibodies may be indirectly conjugated with radiometal ions through the use of bifunctional chelating agents that are covalently linked to the antibodies. Chelating agents may be attached through amities (Meares et al. (1984) Anal. Biochem. 142:68-78); sulfhydral groups (Koyama (1994) Chem. Abstr. 120:217-262) of amino acid residues and carbohydrate groups (Rodwell et al. (1986) PNAS USA 83:2632-2636; Quadri et al. (1993) Nucl. Med. Biol. 20:559-570).

Further, the antibodies or fragments thereof of the present disclosure may be conjugated to a therapeutic agent. Suitable therapeutic agents include taxol, cytochalasin B, gramicidin D, ethidium bromide, emetine, mitomycin, etoposide, tenoposide, vincristine, vinblastine, colchicin, doxorubicin, daunorubicin, dihydroxy anthracin dione, mitoxantrone, mithramycin, actinomycin D, 1-dehydrotestosterone, glucocorticoids, procaine, tetracaine, lidocaine, propranolol, and puromycin, antimetabolites (such as methotrexate, 6-mercaptopurine, 6-thioguanine, cytarabine, fludarabin, 5-fluorouracil, decarbazine, hydroxyurea, asparaginase, gemcitabine, cladribine), alkylating agents (such as mechlorethamine, thiorphan, chlorambucil, melphalan, carmustine (BiCNU), lomustine (CCNU), cyclophosphamide, busulfan, dibromomannitol, streptozotocin, dacarbazine (DTIC), procarbazine, mitomycin C, cisplatin and other platinum derivatives, such as carboplatin), antibiotics (such as dactinomycin (formerly actinomycin), bleomycin, daunorubicin (formerly daunomycin), doxorubicin, idarubicin, mithramycin, mitomycin, mitoxantrone, plicamycin, anthramycin (AMC)), diphtheria toxin and related molecules (such as diphtheria A chain and active fragments thereof and hybrid molecules), ricin toxin (such as ricin A or a deglycosylated ricin A chain toxin), cholera toxin, a Shiga-like toxin (SLT-I, SLT-II, SLT-IIV), LT toxin, C3 toxin, Shiga toxin, pertussis toxin, tetanus toxin, soybean Bowman-Birk protease inhibitor, Pseudomonas exotoxin, alorin, saporin, modeccin, gelanin, abrin A chain, modeccin A chain, alpha-sarcin, Aleurites fordii proteins, dianthin proteins, Phytolacca americana proteins (PAPI, PAPII, and PAP-S), Momordica charantia inhibitor, curcin, crotin, Sapaonaria officinalis inhibitor, gelonin, mitogellin, restrietocin, phenomycin, enomycin toxins and mixed toxins.

Additional suitable conjugated molecules include ribonuclease (RNase), DNase I, an antisense nucleic acid, an inhibitory RNA molecule such as a siRNA molecule, an immunostimulatory nucleic acid, aptamers, ribozymes, triplex forming molecules, and external guide sequences. Aptamers are small nucleic acids ranging from 15-50 bases in length that fold into defined secondary and tertiary structures, such as stem-loops or G-quartets, and can bind small molecules, such as ATP (U.S. Pat. No. 5,631,146) and theophiline (U.S. Pat. No. 5,580,737), as well as large molecules, such as reverse transcriptase (U.S. Pat. No. 5,786,462) and thrombin (U.S. Pat. No. 5,543,293). Ribozymes are nucleic acid molecules that are capable of catalyzing a chemical reaction, either intramolecularly or intermolecularly. Ribozymes typically cleave nucleic acid substrates through recognition and binding of the target substrate with subsequent cleavage. Triplex forming function nucleic acid molecules can interact with double-stranded or single-stranded nucleic acid by forming a triplex, in which three strands of DNA form a complex dependent on both Watson-Crick and Hoogsteen base-pairing. Triplex molecules can bind target regions with high affinity and specificity.

The functional nucleic acid molecules may act as effectors, inhibitors, modulators, and stimulators of a specific activity possessed by a target molecule, or the functional nucleic acid molecules may possess a de novo activity independent of any other molecules.

The therapeutic agents can be linked to the antibody directly or indirectly, using any of a large number of available methods. For example, an agent can be attached at the hinge region of the reduced antibody component via disulfide bond formation, using cross-linkers such as N-succinyl 3-(2-pyridyldithio) propionate (SPDP), or via a carbohydrate moiety in the Fc region of the antibody (Yu et al. 1994 Int. J. Cancer 56:244; Upeslacis et al., “Modification of Antibodies by Chemical Methods,” in Monoclonal antibodies: principles and applications, Birch et al. (eds.), pages 187-230 (Wiley-Liss, Inc. 1995); Price, “Production and Characterization of Synthetic Peptide-Derived Antibodies,” in Monoclonal antibodies: Production, engineering and clinical application, Ritter et al. (eds.), pages 60-84 (Cambridge University Press 1995)).

Techniques for conjugating therapeutic agents to antibodies are well known (Amon et al. “Monoclonal Antibodies For Immunotargeting Of Drugs In Cancer Therapy,” in Monoclonal Antibodies And Cancer Therapy; Reisfeld et al. (eds.), pp. 243-56 (Alan R. Liss, Inc. 1985); Hellstrom et al. “Antibodies For Drug Delivery,” in Controlled Drug Delivery (2nd Ed.); Robinson et al. (eds.), pp. 623-53 (Marcel Dekker, Inc. 1987); Thorpe “Antibody Carriers Of Cytotoxic Agents In Cancer Therapy: A Review,” in Monoclonal Antibodies ‘84: Biological And Clinical Applications, Pinchera et al. (eds.), pp. 475-506 (1985); “Analysis, Results, And Future Prospective Of The Therapeutic Use Of Radiolabeled Antibody in Cancer Therapy,” in Monoclonal Antibodies For Cancer Detection And Therapy, Baldwin et al. (eds.), pp. 303-16 (Academic Press 1985), and Thorpe et al. “The Preparation And Cytotoxic Properties Of Antibody-Toxin Conjugates,” (1982) Immunol. Rev. 62:119-58).

The antibodies disclosed herein or antigen-binding regions thereof can be linked to another functional molecule such as another antibody or ligand for a receptor to generate a bi-specific or multi-specific molecule that binds to at least two or more different binding sites or target molecules. Linking of the antibody to one or more other binding molecules, such as another antibody, antibody fragment, peptide or binding mimetic, can be done, for example, by chemical coupling, genetic fusion, or noncovalent association. Multi-specific molecules can further include a third binding specificity, in addition to the first and second target epitope.

Bi-specific and multi-specific molecules can be prepared using methods known in the art. For example, each binding unit of the hi-specific molecule can be generated separately and then conjugated to one another. When the binding molecules are proteins or peptides, a variety of coupling or cross-linking agents can be used for covalent conjugation. Examples of cross-linking agents include protein A, carbodiimide, N-succinimidyl-S-acetyl-thioacetate (SATA), 5,5’-dithiobis(2-nitroberizoic acid) (DTNB), o-phenylenedimaleimide (oPDM), N-succinimidyl-3-(2-pyridyldithio) propionate (SPDP), and sulfosuccinimidyl 4-(N-maleimidomethyl)cyclohaxane-I-carboxylate (sulfo-SMCC) (Karpovsky et al. (1984) J. Exp. Med. 160:1686; Liu et al. (1985) Proc. Natl. Acad. Sci. USA 82:8648). When the binding molecules are antibodies, they can be conjugated by sulfhydryl bonding of the C-terminus hinge regions of the two heavy chains.

The antibodies or fragments thereof of the present disclosure may be linked to a moiety that is toxic to a cell to which the antibody is bound to form “depleting” antibodies. These antibodies are particularly useful in applications where it is desired to deplete an NK cell.

The antibodies disclosed herein may also be attached to solid supports, which are particularly useful for immunoassays or purification of the target antigen. Such solid supports include, but are not limited to, glass, cellulose, polyacrylamide, nylon, polystyrene, polyvinyl chloride or polypropylene.

The antibodies also can be bound to many different carriers. Thus, this disclosure also provides compositions containing the antibodies and another substance, active or inert. Examples of well-known carriers include glass, polystyrene, polypropylene, polyethylene, dextran, nylon, amylase, natural and modified cellulose, polyacrylamide, agarose, and magnetite. The nature of the carrier can be either soluble or insoluble for purposes disclosed herein. Those skilled in the art will know of other suitable carriers for binding monoclonal antibodies, or will be able to ascertain such, using routine experimentation.

As used herein, the term “antibody derivative”, comprises a full-length antibody or a fragment of an antibody, wherein one or more of the amino acids are chemically modified by alkylation, pegylation, acylation, ester formation or amide formation or the like, e.g., for linking the antibody to a second molecule. This includes, but is not limited to, pegylated antibodies, cysteine-pegylated antibodies, and variants thereof.

As used herein, the term “immunoconjugate” comprises an antibody or an antibody derivative associated with or linked to a second agent, such as a cytotoxic agent, a detectable agent, a radioactive agent, a targeting agent, a human antibody, a humanized antibody, a chimeric antibody, a synthetic antibody, a semisynthetic antibody, or a multispecific antibody.

Examples of suitable fluorescent labels include, but are not limited to, fluorescein, rhodamine, tetramethylrhodamine (TRITC), eosin, erythrosin, coumarin, methyl-coumarins, pyrene, Malacite green, stilbene, Lucifer Yellow, Cascade Blue™, and Texas Red. Other suitable optical dyes are described in the Haugland, Richard P. (1996) Handbook of Fluorescent Probes and Research Chemicals (6th ed.).

In another aspect, the fluorescent label is functionalized to facilitate covalent attachment to a cellular component present in or on the surface of the cell or tissue such as a cell surface marker. Suitable functional groups, include, but are not limited to, isothiocyanate groups, amino groups, haloacetyl groups, maleimides, succinimidyl esters, and sulfonyl halides, all of which may be used to attach the fluorescent label to a second molecule. The choice of the functional group of the fluorescent label will depend on the site of attachment to either a linker, the agent, the marker, or the second labeling agent.

“Immune response” broadly refers to the antigen-specific responses of lymphocytes to foreign substances. Any substance that can elicit an immune response is said to be “immunogenic” and is referred to as an “immunogen”. All immunogens are antigens, however, not all antigens are immunogenic. An immune response of this disclosure can be humoral (via antibody activity) or cell-mediated (via T cell activation).

The term “modulate an immune response” includes inducing (increasing, eliciting) an immune response; and reducing (suppressing) an immune response. An immunomodulatory method (or protocol) is one that modulates an immune response in a subject.

An “HMG domain” or “high mobility group (HMG) box domain” refers to an amino acid sequence that is involved in binding DNA (Stros et al., Cell Mol Life Sci. 64 (19-20): 2590-606 (2007)). In one embodiment, the structure of the HMG-box domain consists of three helices in an irregular array. In another embodiment, an HMG-box domain enables a protein to bind non-B-type DNA conformations (kinked or unwound) with high affinity. HMG-box domains can be found in high mobility group proteins, which are involved in the regulation of DNA-dependent processes such as transcription, replication and DNA repair, all of which require changing the conformation of chromatin (Thomas (2001) Biochem. Soc. Trans. 29 (Pt 4): 395-401).

The compositions used in accordance with the disclosure can be packaged in dosage unit form for ease of administration and uniformity of dosage. The term “unit dose” or “dosage” refers to physically discrete units suitable for use in a subject, each unit containing a predetermined quantity of the composition calculated to produce the desired responses in association with its administration, i.e., the appropriate route and regimen. The quantity to be administered, both according to number of treatments and unit dose, depends on the result and/or protection desired. Precise amounts of the composition also depend on the judgment of the practitioner and are peculiar to each individual. Factors affecting dose include physical and clinical state of the subject, route of administration, intended goal of treatment (alleviation of symptoms versus cure), and potency, stability, and toxicity of the particular composition. Upon formulation, solutions are administered in a manner compatible with the dosage formulation and in such amount as is therapeutically or prophylactically effective. The formulations are easily administered in a variety of dosage forms, such as the type of injectable solutions described herein.

The term “contacting” means direct or indirect binding or interaction between two or more. A particular example of direct interaction is binding. A particular example of an indirect interaction is where one entity acts upon an intermediary molecule, which in turn acts upon the second referenced entity. Contacting as used herein includes in solution, in solid phase, ex vivo, in a cell and. Contacting can be referred to as administering, or administration.

As used herein, a “recombinant peptide” refers to a peptide produced in a host cell (e.g., E. coli, yeast) using exogenous/recombinant nucleic acids. As used herein, a “synthetic peptide” refers to a peptide synthesized by synthetic or chemical means, without using a host cell. While a recombinant peptide may have post-translational modifications, a synthetic peptide does not have any post-translational modifications.

In certain embodiments, the biofilm is derived from (i.e., produced by) a gram negative or a gram-positive biofilm producing bacteria. In certain embodiments, the biofilm is derived from (i.e., produced by) a Nontuberculous mycobacterium (NTM) species.

In certain embodiments, the biofilm comprises a DNABII protein. In a further embodiment, the biofilm comprises a histone-like protein from E. coli strain U93 (HU) or an integration host factor (IHF) binding protein.

In certain embodiments, the DNABII peptide is an IHF peptide. Additionally, or alternatively, the DNABII peptide is an HU peptide. In certain embodiments, the tip region of DNABII peptide is the tip region of IHFA and/or the tip region of IHFB. In a further embodiment, the tip region of DNABII peptide is an IHFA tip region conjugated directly or indirectly (for example via a linker) to an IHFB tip region. In yet a further embodiment, the tip region of DNABII peptide is the IhfA5-mIhfB4NTHI tip chimeric peptide.

As used herein, the term “EC₅₀” refers to the concentration of an antibody or a fragment thereof which induces a response (for example, binding between the antibody or a fragment thereof and its target) halfway between the baseline and maximum after a specified exposure time.

Several parameters are used herein to describe the binding and unbinding reaction of receptor (R, such as an antibody or a fragment thereof) and ligand (L, such as the target of the antibody or a fragment thereof) molecules, which is formalized as: R+L═RL. The reaction is characterized by the on-rate constant kₒₙ and the off-rate constant k_{off}, which have units of M⁻¹ s⁻¹ and s⁻¹, respectively. In equilibrium, the forward binding transition R+L→RL should be balanced by the backward unbinding transition RL→R+L. That is kₒₙ [R][L]=K_{off} [RL] where [R], [L] and [RL] represent the concentration of unbound free receptors, the concentration of unbound free ligand and the concentration of receptor-ligand complexes. Further, the equilibrium dissociation constant “K_{D}” can be calculated as k_{off}/kₒₙ which is [R]×[L]/[RL], while the equilibrium association constant “K_{A}” can be calculated as kₒₙ/k_{off} which is [RL]/([R]×[L]).

As used herein, “Nontuberculous mycobacterium (NTM)” refers to any mycobacterium species other than M. tuberculosis. In some embodiments, NTM species include M. abscessus (Mab), M. avium (Mav), M. intracellulare, or M. chimaera.

As used herein, “antibiotic resistance” or “resistance” refers to a resistance of a micro-organism against an antibiotic agent when the antibiotic agent is administered to a subject in need thereof in a dose that is sufficient to successfully eliminate the micro-organism in its non-resistant form. Biofilms are known to have high antibiotic resistance.

As used herein, “sensitizing a biofilm to an antibiotic agent” means reducing the antibiotic resistance of a biofilm, rendering the resident micro-organisms within the biofilm susceptible to antibiotic agents.

### MODES FOR CARRYING OUT THE DISCLOSURE

Applicant has discovered that an antibody or an antigen-binding fragment thereof that binds to a tip region of a DNABII peptide as described herein shows unexpected effectiveness against infections and biofilms caused by a Nontuberculous mycobacterium (NTM) species, either by enhancing antibiotic sensitivity or immune system clearance in infected individuals. The methods disclosed herein can benefit patients by reducing either length of antibacterial treatment or amount of antibiotic required.

### Methods for Preventing or Treating a Nontuberculous mycobacterium (NTM) Infection

An aspect of the disclosure is directed to a method for preventing or treating an infection caused by a Nontuberculous mycobacterium (NTM) species in a subject comprising, or consisting essentially of, or yet further consisting of, administering to the subject an effective amount of an antibody or an antigen-binding fragment thereof, wherein the antibody or antigen-binding fragment thereof recognizes and binds to a tip region of a DNABII peptide, or a tip chimer (for example an IhfA5-mIhfB4_{NTHI} tip chimer as described herein). Also provided is a composition comprising one or more antibodies or antigen-binding fragments thereof that recognize and bind the IhfA5-mIhfB4_{NTHI} tip chimer.

In some embodiments, the antibody or antigen-binding fragment thereof that binds to a tip region of a DNABII peptide or a tip chimer is an antibody or antigen-binding fragment thereof comprises:
(i) a heavy chain (HC) immunoglobulin variable domain sequence comprising, or consisting essentially of, or yet further consisting of a sequence of amino acid (aa) 25 to aa 144 of SEQ ID NO: 21 or an equivalent thereof, and
(ii) a light chain (LC) immunoglobulin variable domain sequence comprising, or consisting essentially of, or yet further consisting of a sequence of aa 21 to aa 132 of SEQ ID NO: 22 or an equivalent thereof;
or
wherein the antibody or antigen-binding fragment thereof comprises:
(i) a heavy chain (HC) immunoglobulin variable domain sequence comprising, or consisting essentially of, or yet further consisting of a sequence of aa 25 to aa 144 of SEQ ID NO: 24 or an equivalent thereof, and
(ii) a light chain (LC) immunoglobulin variable domain sequence comprising, or consisting essentially of, or yet further consisting of a sequence of aa 21 to aa 132 of SEQ ID NO: 25 or an equivalent thereof.

In some embodiments, the antibody or antigen-binding fragment thereof that binds to a tip region of a DNABII peptide comprises:

a heavy chain complementarity-determining region 1 (CDRH1) comprising, or consisting essentially of, or yet further consisting of a sequence of GFTFRTY (SEQ ID NO: 58) (aa 50 to aa 56 of SEQ ID NO: 9 or 10 or 11 or 24), a heavy chain complementarity-determining region 2 (CDRH2) comprising, or consisting essentially of, or yet further consisting of a sequence of GSDRRH (SEQ ID NO: 59) (aa 76 to aa 81 of SEQ ID NO: 9 or 10 or 11 or 24), a heavy chain complementarity-determining region 3 (CDRH3) comprising, or consisting essentially of, or yet further consisting of a sequence of VGPYDGYYGEFDY (SEQ ID NO: 60) (aa 121 to aa 133 of SEQ ID NO: 9 or 10 or 11 or 24), a light chain complementarity-determining region 1 (CDRL1) comprising, or consisting essentially of, or yet further consisting of a sequence of QSLLDSDGKTF (SEQ ID NO: 61) (aa 47 to aa 57 of SEQ ID NO: 15 or 16 or 17 or 25), a light chain complementarity-determining region 2 (CDRL2) comprising, or consisting essentially of, or yet further consisting of a sequence of LVS (aa 75 to aa 77 of SEQ ID NO: 15 or 16 or 17 or 25), and a light chain complementarity-determining region 3 (CDRL3) comprising, or consisting essentially of, or yet further consisting of a sequence of WQGTHFP (SEQ ID NO: 62) (aa 114 to aa 120 of SEQ ID NO: 15 or 16 or 17 or 25).

In some embodiments, the antibody or antigen-binding fragment thereof that binds to a tip region of a DNABII peptide comprises:
a heavy chain complementarity-determining region 1 (CDRH1) comprising, or consisting essentially of, or yet further consisting of a sequence of GFTFSRYG (SEQ ID NO: 63) (aa 50 to aa 57 of SEQ ID NO: 12 or 13 or 14),
a heavy chain complementarity-determining region 2 (CDRH2) comprising, or consisting essentially of, or yet further consisting of a sequence of ISSGGSYT (SEQ ID NO: 64) (aa 75 to aa 82 of SEQ ID NO: 12 or 13 or 14),
a heavy chain complementarity-determining region 3 (CDRH3) comprising, or consisting essentially of, or yet further consisting of a sequence of ERHGGDGYWYFDV (SEQ ID NO: 65) (aa 121 to aa 133 of SEQ ID NO: 12 or 13 or 14),
a light chain complementarity-determining region 1 (CDRL1) comprising, or consisting essentially of, or yet further consisting of a sequence of QSLLDSDGKTF (SEQ ID NO: 61) (aa 47 to aa 57 of SEQ ID NO: 15 or 16 or 17 or 25),
a light chain complementarity-determining region 2 (CDRL2) comprising, or consisting essentially of, or yet further consisting of a sequence of LVS (aa 75 to aa 77 of SEQ ID NO: 15 or 16 or 17 or 25), and
a light chain complementarity-determining region 3 (CDRL3) comprising, or consisting essentially of, or yet further consisting of a sequence of WQGTHFPYT (SEQ ID NO: 66) (aa 114 to aa 122 of SEQ ID NO: 15 or 16 or 17 or 25).

In some embodiments, the antibody or antigen-binding fragment thereof that binds to a tip region of a DNABII peptide comprises: a heavy chain (HC) immunoglobulin variable domain comprising the amino acid sequence of amino acids 25 to 144 of SEQ ID NO: 9, and/or a light chain (LC) immunoglobulin variable domain comprising the amino acid sequence of amino acids 21 to 132 of SEQ ID NO: 15, SEQ ID NO: 16, or SEQ ID NO: 17.

In some embodiments, the antibody or antigen-binding fragment thereof that binds to a tip region of a DNABII peptide or a tip chimer comprises or consists essentially of, or yet further consists of or consists essentially of, or yet further consists of: a heavy chain (HC) immunoglobulin variable domain sequence comprising, or consisting essentially of, or yet further consisting of a sequence selected from the group of aa 25 to aa 144 of SEQ ID NO: 21 or 24, or an equivalent of each thereof, and/or a light chain (LC) immunoglobulin variable domain sequence comprising, or consisting essentially of, or yet further consisting of a sequence selected from the group of aa 21 to aa 132 of SEQ ID NO: 22 or 25 or an equivalent of each thereof. In certain embodiments, the antibody or antigen-binding fragment thereof binds to a DNABII peptide (such as the tip region of the DNABII peptide including but not limited to: a tip region of IHF or HU, a tip region of IHFA or IHFB, and/or the tip-chimeric peptide IhfA5-mIhfB4_{NTHI}). In one embodiment, the antibody or antigen-binding fragment thereof binds to a tip-chimeric peptide IhfA5-mIhfB4_{NTHI} as described herein. In some embodiments, a tip-chimeric peptide IhfA5-mIhfB4_{NTHI} comprises or consists essentially of, or yet further consists of an amino acid sequence selected from SEQ ID NOs: 26-28.

In some embodiments, the antibody or antigen-binding fragment thereof that binds to a tip region of a DNABII peptide comprises or consists essentially of, or yet further consists of: any one or any two or three heavy chain (HC) CDR comprising, or consisting essentially of, or yet further consisting of a sequence selected from SEQ ID NO: 9-14, or an equivalent of each thereof, and/or any one or any two or three light chain (LC) CDR comprising, or consisting essentially of, or yet further consisting of a sequence selected from 15-20 or an equivalent of each thereof. In certain embodiments, the antibody or antigen-binding fragment thereof binds to a DNABII peptide (such as the tip region of the DNABII peptide including but not limited to a tip region of IHF or HU, a tip region of IHFA or IHFB, and/or a tip-chimeric peptide IhfA5-mIhfB4_{NTHI} as described herein). In one embodiment, the antibody or antigen-binding fragment thereof binds to a tip-chimeric peptide IhfA5-mIhfB4_{NTHI} as described herein. In some embodiments, a tip-chimeric peptide IhfA5-mIhfB4_{NTHI} comprises or consists essentially of, or yet further consists of an amino acid sequence selected from SEQ ID NOs: 26-28.

In some embodiments, the antibody or antigen-binding fragment thereof that binds to a tip region of a DNABII peptide comprises or consists essentially of, or yet further consists of: all three heavy chain (HC) CDR comprising, or consisting essentially of, or yet further consisting of a sequence selected from SEQ ID NO: 9-11, or an equivalent of each thereof, and/or all three light chain (LC) CDR comprising, or consisting essentially of, or yet further consisting of a sequence selected from 15-17 or an equivalent of each thereof. In certain embodiments, the antibody or antigen-binding fragment thereof binds to a DNABII peptide (such as the tip region of the DNABII peptide including but not limited to a tip region of IHF or HU, a tip region of IHFA or IHFB, and/or a tip-chimeric peptide IhfA5-mIhfB4_{NTHI}). In one embodiment, the antibody or antigen-binding fragment thereof binds to a tip-chimeric peptide IhfA5-mIhfB4_{NTHI}. In some embodiments, a tip-chimeric peptide IhfA5-mIhfB4_{NTHI} comprises or consists essentially of, or yet further consists of an amino acid sequence selected from SEQ ID NOs: 3-5.

In some embodiments, the antibody or antigen-binding fragment thereof that binds to a tip region of a DNABII peptide comprises or consists essentially of, or yet further consists of: all three heavy chain (HC) CDR comprising, or consisting essentially of, or yet further consisting of a sequence selected from SEQ ID NO: 12-14, or an equivalent of each thereof, and/or all three light chain (LC) CDR comprising, or consisting essentially of, or yet further consisting of a sequence selected from 18-20 or an equivalent of each thereof. In certain embodiments, the antibody or antigen-binding fragment thereof binds to a DNABII peptide (such as the tip region of the DNABII peptide including but not limited to a tip region of IHF or HU, a tip region of IHFA or IHFB, and/or the tip-chimeric peptide IhfA5-mIhfB4_{NTHI}). In one embodiment, the antibody or antigen-binding fragment thereof binds to a tip-chimeric peptide IhfA5-mIhfB4_{NTHI}. In some embodiments, a tip-chimeric peptide IhfA5-mIhfB4_{NTHI} comprises or consists essentially of, or yet further consists of an amino acid sequence selected from SEQ ID NOs: 26-28.

In some embodiments, the equivalent to an amino acid sequence comprises a polypeptide having at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% amino acid identity to the amino acid, or wherein an equivalent to the amino acid sequence comprises a polypeptide that is encoded by a polynucleotide that hybridizes under conditions of high stringency to the complement of the polynucleotide encoding the amino acid sequence.

Table 1 provides relative biofilm disruption data using humanized monoclonal antibodies designed to target IhfA5-mIhfB4_{NTHI} tip-chimeric peptide. Biofilm disruption: NTHI 86-028NP colonies were collected from overnight culture on chocolate agar and suspended in brain heart infusion broth supplemented with 2 μg β-NAD and heme per ml medium (sBHI). The optical density at 490 nm was then adjusted to 0.65 and the culture diluted 1:6 in sBHI prior to incubation at 37° C. with 5% CO₂ for 3 hr, static. Next, the culture was diluted 1:2500 in fresh sBHI and 200 μl of the suspension aliquoted into each well of an 8-well chamber slide. The slide was then incubated at 37° C. with 5% CO₂ for 3 hr, static. After 16 hr, 200 μl fresh sBHI was added to each well, and the slide incubated an additional 8 hr. At this time point, medium was aspirated from each well and 5 ug monoclonal antibody added per well. The biofilms were incubated an additional 16 hr. Biofilms were then washed and stained with FM1-43FX bacterial cell membrane stain (Invitrogen) and fixed overnight at 4° C. in 16% paraformaldehyde, 2.5% glutaraldehyde, 4.0% acetic acid in 0.1 M phosphate buffer (pH 7.4). Fixative was aspirated and 200 μL Hank's Balanced Salt Solution was added to each well prior to viewing of biofilms on a Zeiss 800 Meta-laser scanning confocal microscope. Images were compiled with Zeiss Zen Black software and biofilm biomass calculated with COMSTAT2.1 software. The KA for the IhfA5-mIhfB4_{NTHI} Tip chimeric peptide (in 1/M) is about 4E+05 to about 2E+08.

TABLE 1
Relative biofilm disruption data using different humanized monoclonal
antibodies designed to target IhfA5-mIhfB4_{NTHI} tip-chimeric peptide

| | | | Biofilm disruption² |
|---|---|---|---|
| | | | (NCH) |
| | | | Value indicates |
| | | | remaining biomass |
| | | | (μm³/μm²) |
| Peptide | Heavy and light chain combination | | Nontypeable |
| target | Heavy chain | Light chain | Haemophilus influenzae |
| IhfA5- | HC1 (SEQ ID NO: 9) | LC1 (SEQ ID NO: 15) | 4.41 |
| mIhfB4 | HC1 (SEQ ID NO: 9) | LC2 (SEQ ID NO: 16) | 12.9 |
| Tip | HC1 (SEQ ID NO: 9) | LC3 (SEQ ID NO: 17) | 8.3 |
| chimeric | HC2 (SEQ ID NO: 10) | LC1 (SEQ ID NO: 15) | 7.1 |
| peptide | HC2 (SEQ ID NO: 10) | LC2 (SEQ ID NO: 16) | 6.9 |
| | HC2 (SEQ ID NO: 10) | LC3 (SEQ ID NO: 17) | 12.1 |
| | HC3 (SEQ ID NO: 11) | LC1 (SEQ ID NO: 15) | 7.4 |
| | HC3 (SEQ ID NO: 11) | LC2 (SEQ ID NO: 16) | 7.6 |
| | HC3 (SEQ ID NO: 11) | LC3 (SEQ ID NO: 17) | 14.1 |

In some embodiments, the antibody or antigen-binding fragment thereof that binds to a tip region of a DNABII peptide comprises or consists essentially of, or yet further consists of a heavy chain sequence comprising SEQ ID NO: 21 and a light chain sequence comprising SEQ ID NO: 22.

In some embodiments, the antibody or antigen-binding fragment thereof that binds to a tip region of a DNABII peptide comprises or consists essentially of, or yet further consists of a heavy chain sequence comprising SEQ ID NO: 9 and a light chain sequence comprising SEQ ID NO: 15.

In some embodiments, the antibody or antigen-binding fragment thereof that binds to a tip region of a DNABII peptide comprises or consists essentially of, or yet further consists of a heavy chain sequence comprising SEQ ID NO: 9 and a light chain sequence comprising SEQ ID NO: 16.

In some embodiments, the antibody or antigen-binding fragment thereof that binds to a tip region of a DNABII peptide comprises or consists essentially of, or yet further consists of a heavy chain sequence comprising SEQ ID NO: 9 and a light chain sequence comprising SEQ ID NO: 17.

In some embodiments, the antibody or antigen-binding fragment thereof that binds to a tip region of a DNABII peptide comprises or consists essentially of, or yet further consists of a heavy chain sequence comprising SEQ ID NO: 10 and a light chain sequence comprising SEQ ID NO: 15.

In some embodiments, the antibody or antigen-binding fragment thereof that binds to a tip region of a DNABII peptide comprises or consists essentially of, or yet further consists of a heavy chain sequence comprising SEQ ID NO: 10 and a light chain sequence comprising SEQ ID NO: 16.

In some embodiments, the antibody or antigen-binding fragment thereof that binds to a tip region of a DNABII peptide comprises or consists essentially of, or yet further consists of a heavy chain sequence comprising SEQ ID NO: 10 and a light chain sequence comprising SEQ ID NO: 17.

In some embodiments, the antibody or antigen-binding fragment thereof that binds to a tip region of a DNABII peptide comprises or consists essentially of, or yet further consists of a heavy chain sequence comprising SEQ ID NO: 11 and a light chain sequence comprising SEQ ID NO: 15.

In some embodiments, the antibody or antigen-binding fragment thereof that binds to a tip region of a DNABII peptide comprises or consists essentially of, or yet further consists of a heavy chain sequence comprising SEQ ID NO: 11 and a light chain sequence comprising SEQ ID NO: 16.

In some embodiments, the antibody or antigen-binding fragment thereof that binds to a tip region of a DNABII peptide comprises or consists essentially of, or yet further consists of a heavy chain sequence comprising SEQ ID NO: 11 and a light chain sequence comprising SEQ ID NO: 17.

In some embodiments, the antibody or antigen-binding fragment thereof that binds to a tip region of a DNABII peptide comprises or consists essentially of, or yet further consists of a heavy chain sequence comprising SEQ ID NO: 12 and a light chain sequence comprising SEQ ID NO: 18.

In some embodiments, the antibody or antigen-binding fragment thereof that binds to a tip region of a DNABII peptide comprises or consists essentially of, or yet further consists of a heavy chain sequence comprising SEQ ID NO: 12 and a light chain sequence comprising SEQ ID NO: 19.

In some embodiments, the antibody or antigen-binding fragment thereof that binds to a tip region of a DNABII peptide comprises or consists essentially of, or yet further consists of a heavy chain sequence comprising SEQ ID NO: 12 and a light chain sequence comprising SEQ ID NO: 20.

In some embodiments, the antibody or antigen-binding fragment thereof that binds to a tip region of a DNABII peptide comprises or consists essentially of, or yet further consists of a heavy chain sequence comprising SEQ ID NO: 13 and a light chain sequence comprising SEQ ID NO: 18.

In some embodiments, the antibody or antigen-binding fragment thereof that binds to a tip region of a DNABII peptide comprises or consists essentially of, or yet further consists of a heavy chain sequence comprising SEQ ID NO: 13 and a light chain sequence comprising SEQ ID NO: 19.

In some embodiments, the antibody or antigen-binding fragment thereof that binds to a tip region of a DNABII peptide comprises or consists essentially of, or yet further consists of a heavy chain sequence comprising SEQ ID NO: 13 and a light chain sequence comprising SEQ ID NO: 20.

In some embodiments, the antibody or antigen-binding fragment thereof that binds to a tip region of a DNABII peptide comprises or consists essentially of, or yet further consists of a heavy chain sequence comprising SEQ ID NO: 14 and a light chain sequence comprising SEQ ID NO: 18.

In some embodiments, the antibody or antigen-binding fragment thereof that binds to a tip region of a DNABII peptide comprises or consists essentially of, or yet further consists of a heavy chain sequence comprising SEQ ID NO: 14 and a light chain sequence comprising SEQ ID NO: 19.

In some embodiments, the antibody or antigen-binding fragment thereof that binds to a tip region of a DNABII peptide comprises or consists essentially of, or yet further consists of a heavy chain sequence comprising SEQ ID NO: 14 and a light chain sequence comprising SEQ ID NO: 20.

Also provided is a method for preventing or treating an infection caused by a Nontuberculous mycobacterium (NTM) species in a subject comprising, or consisting essentially of, or yet further consisting of, administering to the subject an effective amount of an antibody or an antigen-binding fragment thereof, that comprises a heavy chain complementarity-determining region 1 (CDRH1) comprising a sequence of GFTFRTY (SEQ ID NO: 58) (aa 50 to aa 56 of SEQ ID NO: 9); a heavy chain complementarity-determining region 2 (CDRH2) comprising a sequence of GSDRRH (SEQ ID NO: 59) (aa 76 to aa 81 of SEQ ID NO: 9); a heavy chain complementarity-determining region 3 (CDRH3) comprising a sequence of VGPYDGYYGEFDY (SEQ ID NO: 60) (aa 121 to aa 133 of SEQ ID NO: 9); a light chain complementarity-determining region 1 (CDRL1) comprising a sequence of QSLLDSDGKTF (SEQ ID NO: 61) (aa 47 to aa 57 of SEQ ID NO: 15); a light chain complementarity-determining region 2 (CDRL2) comprising a sequence of LVS (aa 75 to aa 77 of SEQ ID NO: 15); and a light chain complementarity-determining region 3 (CDRL3) comprising a sequence of WQGTHFP (SEQ ID NO: 62) (aa 114 to aa 120 of SEQ ID NO: 15)

In one aspect of the disclosed methods, the subject is a mammal such as a pet or animal model or a human patient. The subject can be suffering from an active infection or alternatively, at risk of infection. When administered to an animal, the method is a useful treatment for pets and other non-human animals or mammals, or can serve as a useful animal model to test for combination therapies.

The antibody or antigen-binding fragment thereof for use in the disclosed methods as provided herein can be monospecific or bispecific. In one embodiment, the antibody or antigen-binding fragment thereof is trispecific, or tetraspecific, or pentaspecific. Additionally or alternatively, the antibody is selected from the group of an IgA (such as an IgAQ1 or an IgA2), an IgD, an IgE, an IgG (such as an IgG1, an IgG2, an IgG3, or an IgG4), or an IgM antibody. In one embodiment, the antibody further comprises a constant region selected from the group of: an IgA constant region (such as an IgAQ1 constant region or an IgA2 constant region), an IgD constant region, an IgE constant region, an IgG constant region (such as an IgG1 constant region, an IgG2 constant region, an IgG3 constant region, or an IgG4 constant region) or an IgM constant region. In some embodiments, the constant region of the antibody comprises an amino acid sequence selected from the group of SEQ ID NOs: 23, and 33-40.

In another aspect, the antibodies or antigen-binding fragment thereof can be modified by conventional techniques, that may in one aspect increase the half-life of the antibody, e.g., PEGylation, a PEG mimetic, polysialyation, HESylation or glycosylation.

Methods to make the antibodies and or antigen-binding fragments thereof that bind tip chimer peptides and tail chimer peptides are described in U.S. Pat. No. 11,104,723, which is incorporated herein in its entirety.

In some embodiments, the antigen-binding fragment is selected from the group of Fab, F(ab′)₂, Fab′, scFv, or Fv.

In some embodiments, the method further comprises administering to the subject an effective amount of at least one antibiotic agent (e.g., one, two, three, . . . up to and including all recited antibiotic agents) selected from amikacin, azithromycin, dactinomycin, bleomycin, daunorubicin, doxorubicin, idarubicin, mithramycin, mitomycin, mitoxantrone, plicamycin, methicillin, vancomycin, daptomycin, mupirocin, penicillin, cloxacillin, erythromycin, clarithromycin, roxithromycin, telithromycin, spiramycin, fidaxomicin, rifampin, ethambutol, streptomycin or anthramycin. In one aspect, the effective amount of the antibiotic is administered in an amount that is less that the amount that is administered in the absence of the antibody or the antigen-binding fragment thereof. Non-limiting examples of such amounts are provided herein, e.g., and can range from less than about 20% to less than about 80% of the effective amount when the antibiotic is the sole active agent and ranges in between.

In some embodiments, the NTM species is M. abscessus (Mab), M. avium (Mav), M. intracellulare, or M. chimaera.

In some embodiments, the administration comprises one or more of: inhalation, inhalation by the use of a nebulizer, oral administration, nasal administration, injection and/or topical application.

In some embodiments, the subject is suffering from chronic obstructive pulmonary disease (COPD), primary ciliary dyskinesia, or cystic fibrosis (CF), SARS COV-2, asthma, bacterial pneumonia, respiratory syncytial virus bronchiolitis, influenza virus infection, transfusion-related acute lung injury, mechanical ventilation; sepsis; atherosclerosis; an autoimmune disease selected from systemic lupus erythematosus, rheumatoid arthritis, Type I diabetes mellitus, or small vessel vasculitis; an autoinflammatory disease selected from gout or inflammatory bowel disease, and/or a metabolic disease selected from Type 2 diabetes or obesity. In one aspect, the subject is suffering from cystic fibrosis (CF).

In some embodiments, the effective amount of the antibody or antigen-binding fragment is between 0.2 mg/kg and 30 mg/kg (e.g., 0.2, 0.5, 1, 1.5, 3, 5, 7, 9, 10, 12, 15, 18, 20, 23, 25, 28, 30 mg/kg, or any value therebetween).

In some embodiments, the effective amount of the antibiotic agent is less than 50 mg/kg, e.g., between 0.2 mg/kg and 50 mg/kg (e.g., 0.2, 0.5, 1, 1.5, 3, 5, 7, 9, 10, 12, 15, 20, 25, 30, 35, 40, 45, or 50 mg/kg, or any value therebetween).

Methods for Preventing or Treating a Nontuberculous mycobacterium (NTM) Infection in Combination with an mB Box 97 peptide and/or a DNA-binding agent

In some embodiments, the method further comprises administering to the subject an effective amount of a synthetic or recombinant polypeptide comprising, or consisting essentially of, or yet further consisting of mB Box-97 that consists of the amino acids 90 to 176 from the coding sequence of the native human HMGB1 protein shown as SEQ ID NO: 45 with a cysteine to serine point mutation at amino acid 106, or an equivalent thereof.

As used herein, an equivalent of a synthetic or recombinant mB Box-97 refers to a sequence that is at least about 70%, or alternatively at least about 75%, or at least about 80%, or at least about 85%, or at least about 90%, or at least about 95%, or at least about 98% or at least about 99% identical to the reference recombinant mB Box-97, or the reference synthetic mB Box-97 that in one aspect, retain the mutated amino acid of a cysteine to serine point mutation at amino acid 106. In one aspect, the percent identity is determined using the BLAST alignment program, using default parameters. In particular, preferred programs are BLASTN and BLASTP, using the following default parameters: Genetic code=standard; filter=none; strand=both; cutoff=60; expect=10; Matrix=BLOSUM62; Descriptions=50 sequences; sort by=HIGH SCORE; Databases=non-redundant, GenBank+EMBL+DDBJ+PDB+GenBank CDS translations+SwissProtein+SPupdate+PIR. Details of these programs can be found at the following Internet address: ncbi.nlm.nih.gov/cgi-bin/BLAST.

In some respects, the equivalent of a recombinant or synthetic polypeptide retains the intended function and/or structural characteristics of the mB Box-97polypeptide. In some embodiments, the equivalent of mB-Box-97 includes the recombinant or synthetic mB Box-97 polypeptide that retains the C to S amino acid substitution at amino acid 106, and which further contain independently at least 2, or alternatively at least 3, or alternatively at least 4, or alternatively at least 5, or at least 6, or alternatively at least 7, or alternatively at least 8, or alternatively at least 9 or alternatively at least 10 amino acids at the amino and/or carboxyl terminus of the polypeptide.

In some embodiments, the recombinant or synthetic mB Box-97 HMGB1 polypeptide further comprises or consists essentially of, or yet further consists of one or more linker polypeptides. An example of a peptide linker is one of SEQ ID NOs: 6-8 and 26-30 or PPKGETKKKF (SEQ ID NO: 46) on the amine and/or carboxy terminus.

In some embodiments, the recombinant or synthetic polypeptide consists of SEQ ID NO: 47.

Another aspect of the disclosure, the recombinant or synthetic polypeptide comprising mB Box-97 consists of the amino acids 80 to 176 from the coding sequence of the native human HMGB1 protein shown as SEQ ID NO: 45 with a cysteine to serine point mutation at amino acid 106 or an equivalent thereof. In some embodiments, the synthetic or recombinant polypeptide consists of SEQ ID NO: 48.

In some embodiments, the effective amount of a synthetic or recombinant polypeptide is between 50 nM and 2 μM (e.g., 50 nM, 60 nM, 70 nM, 80 nM, 90 nM, 100 nM, 120 nM, 150 nM, 170 nM, 200 nM, 220 nM, 250 nM, 270 nM, 300 nM, 350 nM, 400 nM, 450 nM, 500 nM, 600 nM, 650 nM, 700 nM, 750 nM, 800 nM, 850 nM, 900 nM, 950 nM, 1000 nM (1 UM), 1050 nM, 1100 nM, 1150 nM, 1200 nM, 1250 nM, 1300 nM, 1350 nM, 1400 nM, 1450 nM, 1500 nM, 1550 nM, 1600 nM, 1650 nM, 1700 nM, 1750 nM, 1800 nM, 1850 nM, 1900 nM, 1950 nM, or 2000 nM (2 μM)).

In some embodiments, the method further comprises administering to the subject an effective amount of a DNA-binding agent, in combination with the antibody or fragment thereof as described herein, or further in combination with the mB Box 97 peptide. Administration of a DNA-binding agent, in combination with the antibody or fragment thereof as described herein, or further in combination with the mB Box 97 peptide as described herein can be done concurrently or sequentially, with the antibody or the antigen-binding fragment thereof, that is being administered prior to or subsequent to the mB Box97 peptide or an equivalent of each thereof, and/or the DNA-binding agent.

In some embodiments, the DNA-binding agent comprises or consists essentially of, or yet further consists of a Histone-like Nucleoid Structuring protein (H-NS), a polyamine, or a polycation. Administration of a DNA-binding agent, in combination with the antibody or fragment thereof as described herein, or further in combination with the mB Box 97 peptide as described herein can be done concurrently or sequentially, with the antibody or the antigent-binding fragment thereof administered prior to or subsequent to the mB Box97 peptide or an equivalent of each thereof, and or the DNA-binding agent.

In some embodiments, the effective amount of a DNA-binding agent is between 50 nM and 2 μM (e.g., 50 nM, 60 nM, 70 nM, 80 nM, 90 nM, 100 nM, 120 nM, 150 nM, 170 nM, 200 nM, 220 nM, 250 nM, 270 nM, 300 nM, 350 nM, 400 nM, 450 nM, 500 nM, 600 nM, 650 nM, 700 nM, 750 nM, 800 nM, 850 nM, 900 nM, 950 nM, 1000 nM (1 μM), 1050 nM, 1100 nM, 1150 nM, 1200 nM, 1250 nM, 1300 nM, 1350 nM, 1400 nM, 1450 nM, 1500 nM, 1550 nM, 1600 nM, 1650 nM, 1700 nM, 1750 nM, 1800 nM, 1850 nM, 1900 nM, 1950 nM, or 2000 nM (2 μM)).

In some embodiments, the DNA-binding agent comprises or consists essentially of, or yet further consists of a Histone-like Nucleoid Structuring protein (H-NS), a polyamine, or a polycation.

In some embodiments, the H-NS is derived from a gram-negative bacterium or a gram-positive bacterium. In some embodiments, the H-NS is derived from a bacterium of a genus Escherichia, Haemophilus, Streptococcus, Mycobacteria, Klebsiella, or Pseudomonas; optionally wherein the H-NS is derived from E. Coli, Nontypeable Haemophilus influenzae (NTHI), S. pneumoniae, K. pneumoniae, Mycobacterium tuberculosis, or Pseudomonas aeruginosa.

In some embodiments, the H-NS comprises or consists essentially of, or yet further consists of an amino acid sequence having at least 60% (e.g., at least 60, 65, 70, 75, 80, 85, 90, 95, 99% or more) identity to an amino acid sequence selected from SEQ ID NOs: 51-56, the equivalent is identical to the reference polypeptide. In one aspect, the percent identity is determined using the BLAST alignment program, using default parameters. In particular, preferred programs are BLASTN and BLASTP, using the following default parameters: Genetic code=standard; filter=none; strand=both; cutoff=60; expect=10; Matrix=BLOSUM62; Descriptions=50 sequences; sort by=HIGH SCORE; Databases=non-redundant, GenBank+EMBL+DDBJ+PDB+GenBank CDS translations+SwissProtein+SPupdate+PIR. Details of these programs can be found at the following Internet address: ncbi.nlm.nih.gov/cgi-bin/BLAST.

In a further aspect, the subject is a mammal, e.g., a mouse, rat, or a human patient. In one aspect of the disclosed methods, the subject is a mammal such as a pet or animal model or a human patient. The subject can be suffering from an active infection or alternatively, at risk of infection. When administered to an animal, the method is a useful treatment for pets and other non-human animals or mammals, or can serve as a useful animal model to test for combination therapies.

### Methods for Sensitizing a Nontuberculous Mycobacterium (NTM) Biofilm to Antibiotics or Disrupting an NTM Biofilm

Another aspect of the disclosure is directed to a method for sensitizing a biofilm to an antibiotic agent or disrupting a biofilm, wherein the biofilm comprises a Nontuberculous mycobacterium (NTM) species, the method comprising, or consisting essentially of, or consisting of contacting the biofilm with an effective amount of an antibody or a fragment thereof, wherein the antibody or antigen-binding fragment thereof recognizes and binds to a tip region of a DNABII peptide, or a tip chimer (for example an IhfA5-mIhfB4_{NTHI} tip chimer as described herein). The contacting can be or.

Also provided is a method for treating a subject infected by a Nontuberculous mycobacterium (NTM) species or suffering from a disease or disorder caused by the NTM species, the method comprising, or consisting essentially of, or consisting of administering to the subject an effective amount of an antibody or an antigen-binding fragment thereof that binds to a tip region of a DNABII peptide and an antibiotic that inhibits the replication or infectivity of the NTM species in the subject, wherein the antibiotic is administered between about 15% to about 75% of the MIC for the antibiotic. Alternatively, the antibiotic is administered at a MIC of between 20% to about 70%, or alternatively at a MIC of between 20% to about 70%, at a MIC of between 25% to about 60%, or at a MIC of between 25% to about 50%, or alternatively at about 25% or about 50% of the MIC of the antibiotic. In one aspect, the antibiotic is azithromycin or amikacin, and the subject is a mammal, e.g., a human patient. In one aspect, the antibiotic is administered to the lungs of the subject by inhalation therapy, e.g., the use of a nebulizer.

In some embodiments of the methods, the antibody or antigen-binding fragment thereof that binds to a tip region of a DNABII peptide or a tip chimer, the antibody or antigen-binding fragment thereof comprises: a heavy chain (HC) immunoglobulin variable domain sequence comprising, or consisting essentially of, or yet further consisting of a sequence of amino acid (aa) 25 to aa 144 of SEQ ID NO: 21 or an equivalent thereof; and a light chain (LC) immunoglobulin variable domain sequence comprising, or consisting essentially of, or yet further consisting of a sequence of aa 21 to aa 132 of SEQ ID NO: 22 or an equivalent thereof, or wherein the antibody or antigen-binding fragment thereof comprises: a heavy chain (HC) immunoglobulin variable domain sequence comprising, or consisting essentially of, or yet further consisting of a sequence of aa 25 to aa 144 of SEQ ID NO: 24 or an equivalent thereof; and a light chain (LC) immunoglobulin variable domain sequence comprising, or consisting essentially of, or yet further consisting of a sequence of aa 21 to aa 132 of SEQ ID NO: 25 or an equivalent thereof.

In some embodiments of the methods, the antibody or antigen-binding fragment thereof that binds to a tip region of a DNABII peptide comprises: a heavy chain complementarity-determining region 1 (CDRH1) comprising, or consisting essentially of, or yet further consisting of a sequence of GFTFRTY (SEQ ID NO: 58) (aa 50 to aa 56 of SEQ ID NO: 9 or 10 or 11 or 24); a heavy chain complementarity-determining region 2 (CDRH2) comprising, or consisting essentially of, or yet further consisting of a sequence of GSDRRH (SEQ ID NO: 59) (aa 76 to aa 81 of SEQ ID NO: 9 or 10 or 11 or 24); a heavy chain complementarity-determining region 3 (CDRH3) comprising, or consisting essentially of, or yet further consisting of a sequence of VGPYDGYYGEFDY (SEQ ID NO: 60) (aa 121 to aa 133 of SEQ ID NO: 9 or 10 or 11 or 24) and a light chain complementarity-determining region 1 (CDRL1) comprising, or consisting essentially of, or yet further consisting of a sequence of QSLLDSDGKTF (SEQ ID NO: 61) (aa 47 to aa 57 of SEQ ID NO: 15 or 16 or 17 or 25); a light chain complementarity-determining region 2 (CDRL2) comprising, or consisting essentially of, or yet further consisting of a sequence of LVS (aa 75 to aa 77 of SEQ ID NO: 15 or 16 or 17 or 25); and a light chain complementarity-determining region 3 (CDRL3) comprising, or consisting essentially of, or yet further consisting of a sequence of WQGTHFP (SEQ ID NO: 62) (aa 114 to aa 120 of SEQ ID NO: 15 or 16 or 17 or 25).

In some embodiments of the methods, the antibody or antigen-binding fragment thereof that binds to a tip region of a DNABII peptide comprises: a heavy chain complementarity-determining region 1 (CDRH1) comprising, or consisting essentially of, or yet further consisting of a sequence of GFTFSRYG (SEQ ID NO: 63) (aa 50 to aa 57 of SEQ ID NO: 12 or 13 or 14); a heavy chain complementarity-determining region 2 (CDRH2) comprising, or consisting essentially of, or yet further consisting of a sequence of ISSGGSYT (SEQ ID NO: 64) (aa 75 to aa 82 of SEQ ID NO: 12 or 13 or 14); a heavy chain complementarity-determining region 3 (CDRH3) comprising, or consisting essentially of, or yet further consisting of a sequence of ERHGGDGYWYFDV (SEQ ID NO: 65) (aa 121 to aa 133 of SEQ ID NO: 12 or 13 or 14); and a light chain complementarity-determining region 1 (CDRL1) comprising, or consisting essentially of, or yet further consisting of a sequence of QSLLDSDGKTF (SEQ ID NO: 61) (aa 47 to aa 57 of SEQ ID NO: 15 or 16 or 17 or 25); a light chain complementarity-determining region 2 (CDRL2) comprising, or consisting essentially of, or yet further consisting of a sequence of LVS (aa 75 to aa 77 of SEQ ID NO: 15 or 16 or 17 or 25); and a light chain complementarity-determining region 3 (CDRL3) comprising, or consisting essentially of, or yet further consisting of a sequence of WQGTHEPYT (SEQ ID NO: 66) (aa 114 to aa 122 of SEQ ID NO: 15 or 16 or 17 or 25).

In some embodiments of the methods, the antibody or antigen-binding fragment thereof that binds to a tip region of a DNABII peptide comprises: a heavy chain (HC) immunoglobulin variable domain comprising the amino acid sequence of amino acids 25 to 144 of SEQ ID NO: 9; and/or a light chain (LC) immunoglobulin variable domain comprising the amino acid sequence of amino acids 21 to 132 of SEQ ID NO: 15, SEQ ID NO: 16, or SEQ ID NO: 17.

In some embodiments of the methods, the antibody or antigen-binding fragment thereof that binds to a tip region of a DNABII peptide or a tip chimer comprises or consists essentially of, or yet further consists of or consists essentially of, or yet further consists of: a heavy chain (HC) immunoglobulin variable domain sequence comprising, or consisting essentially of, or yet further consisting of a sequence selected from the group of aa 25 to aa 144 of SEQ ID NO: 21 or 24, or an equivalent of each thereof, and/or a light chain (LC) immunoglobulin variable domain sequence comprising, or consisting essentially of, or yet further consisting of a sequence selected from the group of aa 21 to aa 132 of SEQ ID NO: 22 or 25 or an equivalent of each thereof. In certain embodiments, the antibody or antigen-binding fragment thereof binds to a DNABII peptide (such as the tip region of the DNABII peptide including but not limited to: a tip region of IHF or HU, a tip region of IHFA or IHFB, and/or the tip-chimeric peptide IhfA5-mIhfB4_{NTHI}). In one embodiment, the antibody or antigen-binding fragment thereof binds to a tip-chimeric peptide IhfA5-mIhfB4_{NTHI} as described herein. In some embodiments of the methods, a tip-chimeric peptide IhfA5-mIhfB4_{NTHI} comprises or consists essentially of, or yet further consists of an amino acid sequence selected from SEQ ID NOs: 26-28.

In some embodiments of the methods, the antibody or antigen-binding fragment thereof that binds to a tip region of a DNABII peptide comprises or consists essentially of, or yet further consists of: any one or any two or three heavy chain (HC) CDR comprising, or consisting essentially of, or yet further consisting of a sequence selected from SEQ ID NO: 9-14, or an equivalent of each thereof, and/or any one or any two or three light chain (LC) CDR comprising, or consisting essentially of, or yet further consisting of a sequence selected from 15-20 or an equivalent of each thereof. In certain embodiments of the methods, the antibody or antigen-binding fragment thereof binds to a DNABII peptide (such as the tip region of the DNABII peptide including but not limited to a tip region of IHF or HU, a tip region of IHFA or IHFB, and/or a tip-chimeric peptide IhfA5-mIhfB4_{NTHI} as described herein). In one embodiment of the methods, the antibody or antigen-binding fragment thereof binds to a tip-chimeric peptide IhfA5-mIhfB4_{NTHI} as described herein. In some embodiments, a tip-chimeric peptide IhfA5-mIhfB4_{NTHI} comprises or consists essentially of, or yet further consists of an amino acid sequence selected from SEQ ID NOs: 26-28.

In some embodiments of the methods, the antibody or antigen-binding fragment thereof that binds to a tip region of a DNABII peptide comprises or consists essentially of, or yet further consists of: all three heavy chain (HC) CDR comprising, or consisting essentially of, or yet further consisting of a sequence selected from SEQ ID NO: 9-11, or an equivalent of each thereof, and/or all three light chain (LC) CDR comprising, or consisting essentially of, or yet further consisting of a sequence selected from 15-17 or an equivalent of each thereof. In certain embodiments of the methods, the antibody or antigen-binding fragment thereof binds to a DNABII peptide (such as the tip region of the DNABII peptide including but not limited to a tip region of IHF or HU, a tip region of IHFA or IHFB, and/or a tip-chimeric peptide IhfA5-mIhfB4_{NTHI}). In one embodiment of the methods, the antibody or antigen-binding fragment thereof binds to a tip-chimeric peptide IhfA5-mIhfB4_{NTHI}. In some embodiments, a tip-chimeric peptide IhfA5-mIhfB4_{NTHI} comprises or consists essentially of, or yet further consists of an amino acid sequence selected from SEQ ID NOs: 3-5.

In some embodiments of the methods, the antibody or antigen-binding fragment thereof that binds to a tip region of a DNABII peptide comprises or consists essentially of, or yet further consists of: all three heavy chain (HC) CDR comprising, or consisting essentially of, or yet further consisting of a sequence selected from SEQ ID NO: 12-14, or an equivalent of each thereof, and/or all three light chain (LC) CDR comprising, or consisting essentially of, or yet further consisting of a sequence selected from 18-20 or an equivalent of each thereof. In certain embodiments, the antibody or antigen-binding fragment thereof binds to a DNABII peptide (such as the tip region of the DNABII peptide including but not limited to a tip region of IHF or HU, a tip region of IHFA or IHFB, and/or the tip-chimeric peptide IhfA5-mIhfB4_{NTHI}). In one embodiment of the methods, the antibody or antigen-binding fragment thereof binds to a tip-chimeric peptide IhfA5-mIhfB4_{NTHI}. In some embodiments of the methods, a tip-chimeric peptide IhfA5-mIhfB4_{NTHI} comprises or consists essentially of, or yet further consists of an amino acid sequence selected from SEQ ID NOs: 26-28.

In some embodiments of the methods, the equivalent to an amino acid sequence comprises a polypeptide having at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% amino acid identity to the amino acid, or wherein an equivalent to the amino acid sequence comprises a polypeptide that is encoded by a polynucleotide that hybridizes under conditions of high stringency to the complement of the polynucleotide encoding the amino acid sequence.

Table 1 referenced above, provides relative biofilm disruption data using humanized monoclonal antibodies designed to target IhfA5-mIhfB4_{NTHI} tip-chimeric peptide. The antibodies disclosed therein are useful in the method of this disclosure, and specific embodiments thereof are provided below.

In some embodiments of the methods, the antibody or antigen-binding fragment thereof that binds to a tip region of a DNABII peptide comprises or consists essentially of, or yet further consists of a heavy chain sequence comprising SEQ ID NO: 21 and a light chain sequence comprising SEQ ID NO: 22.

In some embodiments of the methods, the antibody or antigen-binding fragment thereof that binds to a tip region of a DNABII peptide comprises or consists essentially of, or yet further consists of a heavy chain sequence comprising SEQ ID NO: 9 and a light chain sequence comprising SEQ ID NO: 15.

In some embodiments of the methods, the antibody or antigen-binding fragment thereof that binds to a tip region of a DNABII peptide comprises or consists essentially of, or yet further consists of a heavy chain sequence comprising SEQ ID NO: 9 and a light chain sequence comprising SEQ ID NO: 16.

In some embodiments of the methods, the antibody or antigen-binding fragment thereof that binds to a tip region of a DNABII peptide comprises or consists essentially of, or yet further consists of a heavy chain sequence comprising SEQ ID NO: 9 and a light chain sequence comprising SEQ ID NO: 17.

In some embodiments of the methods, the antibody or antigen-binding fragment thereof that binds to a tip region of a DNABII peptide comprises or consists essentially of, or yet further consists of a heavy chain sequence comprising SEQ ID NO: 10 and a light chain sequence comprising SEQ ID NO: 15.

In some embodiments of the methods, the antibody or antigen-binding fragment thereof that binds to a tip region of a DNABII peptide comprises or consists essentially of, or yet further consists of a heavy chain sequence comprising SEQ ID NO: 10 and a light chain sequence comprising SEQ ID NO: 16.

In some embodiments of the methods, the antibody or antigen-binding fragment thereof that binds to a tip region of a DNABII peptide comprises or consists essentially of, or yet further consists of a heavy chain sequence comprising SEQ ID NO: 10 and a light chain sequence comprising SEQ ID NO: 17.

In some embodiments, the antibody or antigen-binding fragment thereof that binds to a tip region of a DNABII peptide comprises or consists essentially of, or yet further consists of a heavy chain sequence comprising SEQ ID NO: 11 and a light chain sequence comprising SEQ ID NO: 15.

In some embodiments of the methods the antibody or antigen-binding fragment thereof that binds to a tip region of a DNABII peptide comprises or consists essentially of, or yet further consists of a heavy chain sequence comprising SEQ ID NO: 11 and a light chain sequence comprising SEQ ID NO: 16.

In some embodiments, the antibody or antigen-binding fragment thereof that binds to a tip region of a DNABII peptide comprises or consists essentially of, or yet further consists of a heavy chain sequence comprising SEQ ID NO: 11 and a light chain sequence comprising SEQ ID NO: 17.

In some embodiments of the methods, the antibody or antigen-binding fragment thereof that binds to a tip region of a DNABII peptide comprises or consists essentially of, or yet further consists of a heavy chain sequence comprising SEQ ID NO: 12 and a light chain sequence comprising SEQ ID NO: 18.

In some embodiments of the methods, the antibody or antigen-binding fragment thereof that binds to a tip region of a DNABII peptide comprises or consists essentially of, or yet further consists of a heavy chain sequence comprising SEQ ID NO: 12 and a light chain sequence comprising SEQ ID NO: 19.

In some embodiments of the methods, the antibody or antigen-binding fragment thereof that binds to a tip region of a DNABII peptide comprises or consists essentially of, or yet further consists of a heavy chain sequence comprising SEQ ID NO: 12 and a light chain sequence comprising SEQ ID NO: 20.

|0219] In some embodiments of the methods, the antibody or antigen-binding fragment thereof that binds to a tip region of a DNABII peptide comprises or consists essentially of, or yet further consists of a heavy chain sequence comprising SEQ ID NO: 13 and a light chain sequence comprising SEQ ID NO: 18.

In some embodiments of the methods, the antibody or antigen-binding fragment thereof that binds to a tip region of a DNABII peptide comprises or consists essentially of, or yet further consists of a heavy chain sequence comprising SEQ ID NO: 13 and a light chain sequence comprising SEQ ID NO: 19.

In some embodiments of the methods, the antibody or antigen-binding fragment thereof that binds to a tip region of a DNABII peptide comprises or consists essentially of, or yet further consists of a heavy chain sequence comprising SEQ ID NO: 13 and a light chain sequence comprising SEQ ID NO: 20.

In some embodiments of the methods, the antibody or antigen-binding fragment thereof that binds to a tip region of a DNABII peptide comprises or consists essentially of, or yet further consists of a heavy chain sequence comprising SEQ ID NO: 14 and a light chain sequence comprising SEQ ID NO: 18.

In some embodiments of the methods, the antibody or antigen-binding fragment thereof that binds to a tip region of a DNABII peptide comprises or consists essentially of, or yet further consists of a heavy chain sequence comprising SEQ ID NO: 14 and a light chain sequence comprising SEQ ID NO: 19.

In some embodiments of the methods, the antibody or antigen-binding fragment thereof that binds to a tip region of a DNABII peptide comprises or consists essentially of, or yet further consists of a heavy chain sequence comprising SEQ ID NO: 14 and a light chain sequence comprising SEQ ID NO: 20.

Another aspect of the disclosure is directed to a method for sensitizing a biofilm to an antibiotic agent, wherein the biofilm comprises a Nontuberculous mycobacterium (NTM) species, the method comprising, or consisting essentially or, or yet further consisting of contacting the biofilm with an effective amount of an antibody or a fragment thereof that comprises a heavy chain complementarity-determining region 1 (CDRH1) comprising a sequence of GFTFRTY (SEQ ID NO: 58) (aa 50 to aa 56 of SEQ ID NO: 9), a heavy chain complementarity-determining region 2 (CDRH2) comprising a sequence of GSDRRH (SEQ ID NO: 59) (aa 76 to aa 81 of SEQ ID NO: 9), a heavy chain complementarity-determining region 3 (CDRH3) comprising a sequence of VGPYDGYYGEFDY (SEQ ID NO: 60) (aa 121 to aa 133 of SEQ ID NO: 9), a light chain complementarity-determining region 1 (CDRL1) comprising a sequence of QSLLDSDGKTF (SEQ ID NO: 61) (aa 47 to aa 57 of SEQ ID NO: 15), a light chain complementarity-determining region 2 (CDRL2) comprising a sequence of LVS (aa 75 to aa 77 of SEQ ID NO: 15), and a light chain complementarity-determining region 3 (CDRL3) comprising a sequence of WQGTHFP (SEQ ID NO: 62) (aa 114 to aa 120 of SEQ ID NO: 15).

The antibody or antigen-binding fragment for use in the disclosed methods thereof as provided herein may be monospecific or bispecific. In one embodiment, the antibody or antigen-binding fragment thereof is trispecific, or tetraspecific, or pentaspecific. Additionally, or alternatively, the antibody is selected from the group of an IgA (such as an IgAQ1 or an IgA2), an IgD, an IgE, an IgG (such as an IgG1, an IgG2, an IgG3, or an IgG4), or an IgM antibody. In one embodiment, the antibody further comprises a constant region selected from the group of: an IgA constant region (such as an IgAQ1 constant region or an IgA2 constant region), an IgD constant region, an IgE constant region, an IgG constant region (such as an IgG1 constant region, an IgG2 constant region, an IgG3 constant region, or an IgG4 constant region) or an IgM constant region. In some embodiments of the methods, the constant region of the antibody comprises an amino acid sequence selected from the group of SEQ ID NOs: 23, and 33-40.

In another aspect, the antibodies can be modified by conventional techniques, that may in one aspect increase the half-life of the antibody, e.g., PEGylation, a PEG mimetic, polysialyation, HESylation or glycosylation.

Methods to make the antibodies and antigen-binding fragments thereof are known in the art and described in U.S. Pat. No. 11,104,723, which is incorporated herein in its entirety.

In some embodiments of the methods, the antigen-binding fragment is selected from the group of Fab, F(ab′) 2, Fab′, scFv, or Fv.

In some embodiments of the methods, the antibiotic agent comprises amikacin, azithromycin, dactinomycin, bleomycin, daunorubicin, doxorubicin, idarubicin, mithramycin, mitomycin, mitoxantrone, plicamycin, methicillin, vancomycin, daptomycin, mupirocin, penicillin, cloxacillin, erythromycin, clarithromycin, roxithromycin, telithromycin, spiramycin, fidaxomicin, rifampin, ethambutol, streptomycin or anthramycin.

In some embodiments of the methods, the effective amount of an antibody or antigen-binding fragment is between 0.2 mg/kg and 30 mg/kg (e.g., 0.2, 0.5, 1, 1.5, 3, 5, 7, 9, 10, 12, 15, 18, 20, 23, 25, 28, 30 mg/kg, or any value therebetween).

In some embodiments of the methods, the effective amount of an antibiotic agent is less than 50 mg/kg, e.g., between 0.2 mg/kg and 50 mg/kg (e.g., 0.2, 0.5, 1, 1.5, 3, 5, 7, 9, 10, 12, 15, 20, 25, 30, 35, 40, 45, or 50 mg/kg, or any value therebetween).

In some embodiments of the methods, the contacting is or.

In one aspect of the disclosed in vivo methods, the subject is a mammal such as a pet or animal model or a human patient. The subject can be suffering from an active infection or alternatively, at risk of infection. When the method is practiced in vitro, the method is practiced in a cell or tissue culture with animal systems as described herein.

In some embodiments of the methods, a sensitized biofilm can be treated using less antibiotics. For instance, a biofilm that is resistant to antibiotics having an IC50 (half maximal inhibitory concentration) of 100 mg/kg or more would respond to 50 mg/kg antibiotics or less (e.g., 40, 30, 20, 10, 5, 1, 0.1 mg/kg or less antibiotics) after treatment with the claimed antibodies. Thus, the methods can further comprise administration of less that the ICM (minimum inhibitory concentration) of the antibiotic, wherein the amount or effective amount being administered is concurrently with the antibody or antigen-binding fragment thereof or sequentially, wherein the antibody or antigen-binding fragment is administered prior to or alternatively subsequent to the antibody or antigen-binding fragment thereof.

Combination with an mB Box 97 peptide

In some embodiments, the method further comprises contacting the biofilm with an effective amount of a synthetic or recombinant polypeptide comprising, or consisting essentially of, or yet further consisting of mB Box-97 that consists of the amino acids 90 to 176 from the coding sequence of the native human HMGB1 protein shown as SEQ ID NO: 45 with a cysteine to serine point mutation at amino acid 106 or an equivalent thereof.

As used herein, an equivalent of a synthetic or recombinant mB Box-97 refers to a sequence that is at least about 70%, or alternatively at least about 75%, or at least about 80%, or at least about 85%, or at least about 90%, or at least about 95%, or at least about 98% or at least about 99% identical to the reference recombinant mB Box-97, or the reference synthetic mB Box-97 that in one aspect, retain the mutated amino acid of a cysteine to serine point mutation at amino acid 106. In one aspect, the percent identity is determined using the BLAST alignment program, using default parameters. In particular, preferred programs are BLASTN and BLASTP, using the following default parameters: Genetic code=standard; filter=none; strand=both; cutoff=60; expect=10; Matrix=BLOSUM62; Descriptions=50 sequences; sort by=HIGH SCORE; Databases=non-redundant, GenBank+EMBL+DDBJ+PDB+GenBank CDS translations+SwissProtein+SPupdate+PIR. Details of these programs can be found at the following Internet address: ncbi.nlm.nih.gov/cgi-bin/BLAST.

In some respects, the equivalent of a recombinant or synthetic polypeptide retains the intended function and/or structural characteristics of the mB Box-97polypeptide. In some embodiments, the equivalent of mB-Box-97 includes the recombinant or synthetic mB Box-97 polypeptide that retains the C to S amino acid substitution at amino acid 106, and which further contain independently at least 2, or alternatively at least 3, or alternatively at least 4, or alternatively at least 5, or at least 6, or alternatively at least 7, or alternatively at least 8, or alternatively at least 9 or alternatively at least 10 amino acids at the amino and/or carboxyl terminus of the polypeptide.

In some embodiments, the recombinant or synthetic mB Box-97 HMGB1 polypeptide further comprises or consists essentially of, or yet further consists of one or more linker polypeptides. An example of a peptide linker is one of SEQ ID NOs: 6-8 and 26-30 or PPKGETKKKF (SEQ ID NO: 46) on the amine and/or carboxy terminus.

In some embodiments, the recombinant or synthetic polypeptide consists of SEQ ID NO: 47.

In some embodiments, the method further comprises contacting the biofilm with an effective amount of a synthetic or recombinant polypeptide comprising, or consisting essentially of, or yet further consisting of mB Box-97 that consists of the amino acids 80 to 176 from the coding sequence of the native human HMGB1 protein shown as SEQ ID NO: 45 with a cysteine to serine point mutation at amino acid 106 or an equivalent thereof.

In some embodiments, the synthetic or recombinant polypeptide consists of SEQ ID NO: 48.

In some embodiments, the effective amount of a synthetic or recombinant polypeptide is between 50 nM and 2 μM (e.g., 50 nM, 60 nM, 70 nM, 80 nM, 90 nM, 100 nM, 120 nM, 150 nM, 170 nM, 200 nM, 220 nM, 250 nM, 270 nM, 300 nM, 350 nM, 400 nM, 450 nM, 500 nM, 600 nM, 650 nM, 700 nM, 750 nM, 800 nM, 850 nM, 900 nM, 950 nM, 1000 nM (1 μM), 1050 nM, 1100 nM, 1150 nM, 1200 nM, 1250 nM, 1300 nM, 1350 nM, 1400 nM, 1450 nM, 1500 nM, 1550 nM, 1600 nM, 1650 nM, 1700 nM, 1750 nM, 1800 nM, 1850 nM, 1900 nM, 1950 nM, or 2000 nM (2 μM)), wherein the amount or effective amount being administered is concurrently with the antibody or antigen-binding fragment thereof or sequentially, wherein the antibody or antigen-binding fragment is administered prior to or alternatively subsequent to the antibody or antigen-binding fragment thereof.

Combination with a DNA-binding agent

In some embodiments, method further comprises contacting the biofilm with an effective amount of a DNA-binding agent.

In some embodiments, the DNA-binding agent comprises or consists essentially of, or yet further consists of a Histone-like Nucleoid Structuring protein (H-NS), a polyamine, or a polycation.

In some embodiments, the H-NS is derived from a gram-negative bacterium or a gram-positive bacterium. In some embodiments, the H-NS is derived from a bacterium of a genus Escherichia, Haemophilus, Streptococcus, Mycobacteria, Klebsiella, or Pseudomonas; optionally wherein the H-NS is derived from E. Coli, Nontypeable Haemophilus influenzae (NTHI), S. pneumoniae, K. pneumoniae, Mycobacterium tuberculosis, or Pseudomonas aeruginosa.

|0247] In some embodiments, the H-NS comprises or consists essentially of, or yet further consists of an amino acid sequence having at least 60% (e.g., at least 60, 65, 70, 75, 80, 85, 90, 95, 99% or more) identity to an amino acid sequence selected from SEQ ID NOs: 51-56. In one aspect, the percent identity is determined using the BLAST alignment program, using default parameters. In particular, preferred programs are BLASTN and BLASTP, using the following default parameters: Genetic code=standard; filter=none; strand=both; cutoff=60; expect=10; Matrix=BLOSUM62; Descriptions=50 sequences; sort by=HIGH SCORE; Databases=non-redundant, GenBank+EMBL+DDBJ+PDB+GenBank CDS translations+SwissProtein+SPupdate+PIR. Details of these programs can be found at the following Internet address: ncbi.nlm.nih.gov/cgi-bin/BLAST.

In some embodiments, the effective amount of a DNA-binding agent is between 50 nM and 2 μM (e.g., 50 nM, 60 nM, 70 nM, 80 nM, 90 nM, 100 nM, 120 nM, 150 nM, 170 nM, 200 nM, 220 nM, 250 nM, 270 nM, 300 nM, 350 nM, 400 nM, 450 nM, 500 nM, 600 nM, 650 nM, 700 nM, 750 nM, 800 nM, 850 nM, 900 nM, 950 nM, 1000 nM (1 μM), 1050 nM, 1100 nM, 1150 nM, 1200 nM, 1250 nM, 1300 nM, 1350 nM, 1400 nM, 1450 nM, 1500 nM, 1550 nM, 1600 nM, 1650 nM, 1700 nM, 1750 nM, 1800 nM, 1850 nM, 1900 nM, 1950 nM, or 2000 nM (2 μM)), wherein the amount or effective amount being administered is concurrently with the antibody or antigen-binding fragment thereof or sequentially, wherein the antibody or antigen-binding fragment is administered prior to or alternatively subsequent to the antibody or antigen-binding fragment thereof. Administration of a DNA-binding agent, in combination with the antibody or fragment thereof as described herein, or further in combination with the mB Box 95 peptide as described herein can be done concurrently or sequentially.

In a further aspect, the subject to be treated is a mammal, e.g., a mouse, rat, or a human patient.

### Compositions

Compositions are further provided. The compositions comprise a carrier and an antibody, and/or an antigen-binding fragment disclosed herein alone or in combination with an additional agent such as an antibiotic as described herein, and/or an mB Box-97 peptide as described herein, and/or a DNA-binding agent as described herein. The carriers can be one or more of a solid support or a pharmaceutically acceptable carrier. The compositions can further comprise an adjuvant or other components suitable for administrations as vaccines. In one aspect, the compositions are formulated with one or more pharmaceutically acceptable excipients, diluents, carriers and/or adjuvants. In addition, embodiments of the compositions of the present disclosure include an antibody of the disclosure, formulated with one or more pharmaceutically acceptable substances.

For oral preparations, any one or more of an antibody or fragment thereof as described herein can be used alone or in pharmaceutical formulations disclosed herein comprising, or consisting essentially of, the compound in combination with appropriate additives to make tablets, powders, granules or capsules, for example, with conventional additives, such as lactose, mannitol, corn starch or potato starch; with binders, such as crystalline cellulose, cellulose derivatives, acacia, corn starch or gelatins; with disintegrators, such as corn starch, potato starch or sodium carboxymethylcellulose; with lubricants, such as talc or magnesium stearate; and if desired, with diluents, buffering agents, moistening agents, preservatives and flavoring agents. Pharmaceutically compatible binding agents, and/or adjuvant materials can be included as part of the composition. The tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate or Sterotes; a glidant such as colloidal silicon dioxide; a sweetening agent such as sucrose or saccharin; or a flavoring agent such as peppermint, methyl salicylate, or orange flavoring.

Pharmaceutical formulations and unit dose forms suitable for oral administration are particularly useful in the treatment of chronic conditions, infections, and therapies in which the patient self-administers the drug. In one aspect, the formulation is specific for pediatric administration.

The disclosure provides pharmaceutical formulations in which the antibody disclosed herein, alone or in combination with an additional agent such as an antibiotic as described herein, and/or an mB Box peptide as described herein, and/or a DNA-binding agent as described herein, can be formulated into preparations for injection in accordance with the disclosure by dissolving, suspending or emulsifying them in an aqueous or nonaqueous solvent, such as vegetable or other similar oils, synthetic aliphatic acid glycerides, esters of higher aliphatic acids or propylene glycol; and if desired, with conventional additives such as solubilizers, isotonic agents, suspending agents, emulsifying agents, stabilizers and preservatives or other antimicrobial agents. A non-limiting example of such is a antimicrobial agent such as other vaccine components such as surface antigens, e.g., an OMP P5, OMP 26, OMP P2, or Type IV Pilin protein (see Jurcisek and Bakaletz (2007) J. of Bacteriology 189 (10): 3868-3875 and Murphy, T F, Bakaletz, L O and Smeesters, P R (2009) The Pediatric Infectious Disease Journal, 28: S121-S126) and antibacterial agents. For intravenous administration, suitable carriers include physiological bacteriostatic water, Cremophor EL™ (BASF, Parsippany, N.J.), or phosphate buffered saline (PBS). In all cases, a composition for parenteral administration must be sterile and should be fluid to the extent that easy syringability exists.

Aerosol formulations provided by the disclosure can be administered via inhalation and can be propellant or non-propellant based. For example, embodiments of the pharmaceutical formulations disclosed herein comprise a compound disclosed herein formulated into pressurized acceptable propellants such as dichlorodifluoromethane, propane, nitrogen and the like. For administration by inhalation, the compounds can be delivered in the form of an aerosol spray from a pressurized container or dispenser which contains a suitable propellant, e.g., a gas such as carbon dioxide, or a nebulizer. A non-limiting example of a non-propellant is a pump spray that is ejected from a closed container by means of mechanical force (i.e., pushing down a piston with one's finger or by compression of the container, such as by a compressive force applied to the container wall or an elastic force exerted by the wall itself, e.g., by an elastic bladder).

Suppositories disclosed herein can be prepared by mixing a compound disclosed herein with any of a variety of bases such as emulsifying bases or water-soluble bases. Embodiments of this pharmaceutical formulation of a compound disclosed herein can be administered rectally via a suppository. The suppository can include vehicles such as cocoa butter, carbowaxes and polyethylene glycols, which melt at body temperature, yet are solidified at room temperature.

Unit dosage forms for oral or rectal administration, such as syrups, elixirs, and suspensions, may be provided wherein each dosage unit, for example, teaspoonful, tablespoonful, tablet or suppository, contains a predetermined amount of the composition containing one or more compounds disclosed herein. Similarly, unit dosage forms for injection or intravenous administration may comprise a compound disclosed herein in a composition as a solution in sterile water, normal saline or another pharmaceutically acceptable carrier.

Embodiments of the pharmaceutical formulations disclosed herein include those in which an antibody or fragment thereof as disclosed herein is formulated in an injectable composition. Injectable pharmaceutical formulations disclosed herein are prepared as liquid solutions or suspensions; or as solid forms suitable for solution in, or suspension in, liquid vehicles prior to injection. The preparation may also be emulsified or the active ingredient encapsulated in liposome vehicles in accordance with other embodiments of the pharmaceutical formulations disclosed herein.

In an embodiment, an antibody disclosed herein is formulated for delivery by a continuous delivery system. The term “continuous delivery system” is used interchangeably herein with “controlled delivery system” and encompasses continuous (e.g., controlled) delivery devices (e.g., pumps) in combination with catheters, injection devices, and the like, a wide variety of which are known in the art.

Mechanical or electromechanical infusion pumps can also be suitable for use with the present disclosure. Examples of such devices include those described in, for example, U.S. Pat. Nos. 4,692,147; 4,360,019; 4,487,603; 4,360,019; 4,725,852; 5,820,589; 5,643,207; 6,198,966; and the like. In general, delivery of a compound disclosed herein can be accomplished using any of a variety of refillable, pump systems. Pumps provide consistent, controlled release over time. In some embodiments of the methods, a compound disclosed herein is in a liquid formulation in a drug-impermeable reservoir, and is delivered in a continuous fashion to the individual.

In one embodiment, the drug delivery system is an at least partially implantable device. The implantable device can be implanted at any suitable implantation site using methods and devices well known in the art. An implantation site is a site within the body of a subject at which a drug delivery device is introduced and positioned. Implantation sites include, but are not necessarily limited to, a subdermal, subcutaneous, intramuscular, or other suitable site within a subject's body. Subcutaneous implantation sites are used in some embodiments because of convenience in implantation and removal of the drug delivery device.

Drug release devices suitable for use in the disclosure may be based on any of a variety of modes of operation, polymers such as for example poly (glycolide-co-lactide) (PGLA) that is commercially available from a number of vendors, e.g., BioDegmer and Sigma-Aldrich. For example, the drug release device can be based upon a diffusive system, a convective system, or an erodible system (e.g., an erosion-based system). For example, the drug release device can be an electrochemical pump, osmotic pump, an electroosmotic pump, a vapor pressure pump, or osmotic bursting matrix, e.g., where the drug is incorporated into a polymer (e.g., PGLA) and the polymer provides for release of drug formulation concomitant with degradation of a drug-impregnated polymeric material (e.g., a biodegradable, drug-impregnated polymeric material). In other embodiments of the methods, the drug release device is based upon an electrodiffusion system, an electrolytic pump, an effervescent pump, a piezoelectric pump, a hydrolytic system, etc.

Drug release devices based upon a mechanical or electromechanical infusion pump can also be suitable for use with the present disclosure. Examples of such devices include those described in, for example, U.S. Pat. Nos. 4,692,147; 4,360,019; 4,487,603; 4,360,019; 4,725,852; and the like. In general, a subject treatment method can be accomplished using any of a variety of refillable, non-exchangeable pump systems. Pumps and other convective systems may be utilized due to their generally more consistent, controlled release over time. Osmotic pumps are used in some embodiments due to their combined advantages of more consistent controlled release and relatively small size (see, e.g., PCT International Application Publication No. WO 97/27840 and U.S. Pat. Nos. 5,985,305 and 5,728,396). Exemplary osmotically-driven devices suitable for use in the disclosure include, but are not necessarily limited to, those described in U.S. Pat. Nos. 3,760,984; 3,845,770; 3,916,899; 3,923,426; 3,987,790; 3,995,631; 3,916,899; 4,016,880; 4,036,228; 4,111,202; 4,111,203; 4,203,440; 4,203,442; 4,210,139; 4,327,725; 4,627,850; 4,865,845; 5,057,318; 5,059,423; 5,112,614; 5,137,727; 5,234,692; 5,234,693; 5,728,396; and the like. A further exemplary device that can be adapted for the present disclosure is the Synchromed infusion pump (Medtronic).

In some embodiments of the methods, the drug delivery device is an implantable device. The drug delivery device can be implanted at any suitable implantation site using methods and devices well known in the art. As noted herein, an implantation site is a site within the body of a subject at which a drug delivery device is introduced and positioned. Implantation sites include, but are not necessarily limited to a subdermal, subcutaneous, intramuscular, or other suitable site within a subject's body.

Suitable excipient vehicles for a compound disclosed herein are, for example, water, saline, dextrose, glycerol, ethanol, or the like, and combinations thereof. In addition, if desired, the vehicle may contain minor amounts of auxiliary substances such as wetting or emulsifying agents or pH buffering agents. Methods of preparing such dosage forms are known, or will be apparent upon consideration of this disclosure, to those skilled in the art. See, e.g., Remington's Pharmaceutical Sciences, Mack Publishing Company, Easton, Pa., 17th edition, 1985. The composition or formulation to be administered will, in any event, contain a quantity of the compound adequate to achieve the desired state in the subject being treated.

Compositions of the present disclosure include those that comprise a sustained-release or controlled release matrix. In addition, embodiments of the present disclosure can be used in conjunction with other treatments that use sustained-release formulations. As used herein, a sustained-release matrix is a matrix made of materials, usually polymers, which are degradable by enzymatic or acid-based hydrolysis or by dissolution. Once inserted into the body, the matrix is acted upon by enzymes and body fluids. A sustained-release matrix desirably is chosen from biocompatible materials such as liposomes, polylactides (polylactic acid), polyglycolide (polymer of glycolic acid), polylactide co-glycolide (copolymers of lactic acid and glycolic acid), polyanhydrides, poly(ortho) esters, polypeptides, hyaluronic acid, collagen, chondroitin sulfate, carboxcylic acids, fatty acids, phospholipids, polysaccharides, nucleic acids, polyamino acids, amino acids such as phenylatanine, tyrosine, isoleucine, polynucleotides, polyvinyl propylene, polyvinylpyrrolidone and silicone. Illustrative biodegradable matrices include a polylactide matrix, a polyglycolide matrix, and a polylactide co-glycolide (co-polymers of lactic acid and glycolic acid) matrix.

In another embodiment, the polypeptide, antibody or fragment thereof (as well as combination compositions) is delivered in a controlled release system. For example, a compound disclosed herein may be administered using intravenous infusion, an implantable osmotic pump, a transdermal patch, liposomes, or other modes of administration. In one embodiment, a pump may be used (Sefton (1987) CRC Crit. Ref. Biomed. Eng. 14:201; Buchwald et al. (1980) Surgery 88:507; Saudek et al. (1989) N. Engl. J. Med. 321:574). In another embodiment, polymeric materials are used. In yet another embodiment a controlled release system is placed in proximity of the therapeutic target, i.e., the liver, thus requiring only a fraction of the systemic dose. In yet another embodiment, a controlled release system is placed in proximity of the therapeutic target, thus requiring only a fraction of the systemic. Other controlled release systems are discussed in the review by Langer (1990) Science 249:1527-1533.

In another embodiment, the compositions of the present disclosure (as well as combination compositions separately or together) include those formed by impregnation of an inhibiting agent described herein into absorptive materials, such as sutures, bandages, and gauze, or coated onto the surface of solid phase materials, such as surgical staples, zippers and catheters to deliver the compositions. Other delivery systems of this type will be readily apparent to those skilled in the art in view of the instant disclosure.

In various embodiments of the methods, these methods disclosed herein span almost any available method and route suitable for drug delivery, including and ex vivo methods, as well as systemic and localized routes of administration.

### Combination Therapy

The compositions and related methods of the present disclosure may be used in combination with the administration of other therapies. These include, but are not limited to, the administration of DNase enzymes, antibiotics, antimicrobials, anti-infectives, anti-fungals, anti-parasitics, anti-virals, or other antibodies. In one aspect, the antibiotic is selected from amikacin and azithromycin, that in one aspect, are administered at about 50% or alternatively at about 25% of their MIC and in one aspect is converted to use in animals, e.g., human patients.

In some embodiments, the methods and compositions include a deoxyribonuclease (DNase) enzyme that acts synergistically with the anti-DNABII antibody. A DNase is any enzyme that catalyzes the cleavage of phosphodiester linkages in the DNA backbone. Three non-limiting examples of DNase enzymes that are known to target not only cruciform structures, but also a variety of secondary structure of DNA include DNAse I, T4 EndoVII, T7 Endo I, RuvABC, and RusA. In certain embodiments, the effective amount of anti-DNABII antibody needed to destabilize the biofilm is reduced when combined with a DNase. When administered, the DNase can be added directly to the assay or in a suitable buffer known to stabilize the enzyme. The effective Unit dose of DNase and the assay conditions may vary, and can be optimized according to procedures known in the art.

In other embodiments, the methods and compositions can be combined with antibiotics and/or antimicrobials. Antimicrobials are substances that kill or inhibit the growth of microorganisms such as bacteria, fungi, or protozoans. Although biofilms are generally resistant to the actions of antibiotics, compositions and methods described herein can be used to sensitize the infection involving a biofilm to traditional therapeutic methods for treating infections. In other embodiments, the use of antibiotics or antimicrobials in combination with methods and compositions described herein allow for the reduction of the effective amount of the antimicrobial and/or biofilm reducing agent. Some non-limiting examples of antimicrobials and antibiotics useful in combination with methods of the current disclosure include amoxicillin, amoxicillin-clavulanate, cefdinir, azithromycin, and sulfamethoxazole-trimethoprim. The therapeutically effective dose of the antimicrobial and/or antibiotic in combination with the biofilm reducing agent can be readily determined by traditional methods. In some embodiments the dose of the antimicrobial agent in combination with the biofilm reducing agent is the average effective dose which has been shown to be effective in other bacterial infections, for example, bacterial infections wherein the etiology of the infection does not include a biofilm. In other embodiments, the dose is 0.1, 0.15, 0.2, 0.25, 0.30, 0.35, 0.40, 0.45, 0.50, 0.55, 0.60, 0.65, 0.70, 0.75, 0.8, 0.85, 0.9, 0.95, 1.1, 1.2, 1.3, 1.4, 1.5, 1.6, 1.7, 1.8, 1.9, 2.0, 2.5, 3.0 or 5 times the average effective dose. The antibiotic or antimicrobial can be added prior to, concurrent with, or subsequent to the addition of the anti-DNABII antibody.

In other embodiments, the methods and compositions can be combined with antibodies that treat the bacterial infection. One example of an antibody useful in combination with the methods and compositions described herein is an antibody directed against an unrelated outer membrane protein (i.e., OMP P5). Treatment with this antibody alone does not debulk a biofilm. Combined therapy with this antibody and a biofilm reducing agent results in a greater effect than that which could be achieved by either reagent used alone at the same concentration. Other antibodies that may produce a synergistic effect when combined with a biofilm reducing agent or methods to reduce a biofilm include anti-rsPilA anti-OMP26, anti-OMP P2, and anti-whole OMP preparations.

The compositions and methods described herein can be used to sensitize the bacterial infection involving a biofilm to common therapeutic modalities effective in treating bacterial infections without a biofilm but are otherwise ineffective in treating bacterial infections involving a biofilm. In other embodiments, the compositions and methods described herein can be used in combination with therapeutic modalities that are effective in treating bacterial infections involving a biofilm, but the combination of such additional therapy and biofilm reducing agent or method produces a synergistic effect such that the effective dose of either the biofilm reducing agent or the additional therapeutic agent can be reduced. In other instances, the combination of such additional therapy and biofilm reducing agent or method produces a synergistic effect such that the treatment is enhanced. An enhancement of treatment can be evidenced by a shorter amount of time required to treat the infection.

The additional therapeutic treatment can be added prior to, concurrent with, or subsequent to methods or compositions used to reduce the biofilm and can be contained within the same formation/composition or as a separate formulation/composition.

### Kits

Kits containing one or more of the antibodies or fragments thereof or the agents, mB Box-97 peptides, DNA-binding agents, compositions and instructions necessary to perform the and methods as described herein also are disclosed. Accordingly, the disclosure provides kits for performing these methods which may include an antibody, antibody fragment, polypeptide, polynucleotide, vector or host cell, as well as instructions for carrying out the methods disclosed herein such as collecting tissue and/or performing the screen, and/or analyzing the results, and/or administration of an effective amount of an antibody, antibody fragment, polypeptide, polynucleotide, vector or host cell, as defined herein. These can be used alone or in combination with other suitable antimicrobial agents.

For example, a kit can comprise, or alternatively consist essentially of, or yet further consist of any one or more of agents identified above, e.g., antibody, antibody fragment, polypeptide, polynucleotide, vector or host cell, and instructions for use. The kit can further comprise one or more of an adjuvant, an antigenic peptide or an antimicrobial. Examples of carriers include a liquid carrier, a pharmaceutically acceptable carrier, a solid phase carrier, a pharmaceutically acceptable carrier, a pharmaceutically acceptable polymer, a liposome, a micelle, an implant, a stent, a paste, a gel, a dental implant, or a medical implant.

### EXAMPLES

The following examples are intended to illustrate, but not limit the scope of the disclosure.

### Example 1: Materials and Methods

Antibodies. HuTipMab is an IgG isotype and has been described (Kurbatfinski et al., 2022). Human IgG (HuIgG) without preservative was used as the negative isotype control (Thermo Fisher Scientific, Inc., Waltham, MA). HuTipMab antibody is claimed in U.S. Pat. No. 11,104,723 and comprises a heavy chain complementarity-determining region 1 (CDRH1) comprising a sequence of GFTFRTY (SEQ ID NO: 58) (aa 50 to aa 56 of SEQ ID NO: 9); a heavy chain complementarity-determining region 2 (CDRH2) comprising a sequence of GSDRRH (SEQ ID NO: 59) (aa 76 to aa 81 of SEQ ID NO: 9); a heavy chain complementarity-determining region 3 (CDRH3) comprising a sequence of VGPYDGYYGEFDY (SEQ ID NO: 60) (aa 121 to aa 133 of SEQ ID NO: 9); a light chain complementarity-determining region 1 (CDRL1) comprising a sequence of QSLLDSDGKTF (SEQ ID NO: 61) (aa 47 to aa 57 of SEQ ID NO: 15); a light chain complementarity-determining region 2 (CDRL2) comprising a sequence of LVS (aa 75 to aa 77 of SEQ ID NO: 15); and a light chain complementarity-determining region 3 (CDRL3) comprising a sequence of WQGTHFP (SEQ ID NO: 62) (aa 114 to aa 120 of SEQ ID NO: 15).

Antibiotics. Amikacin sulfate salt and azithromycin dihydrate were purchased from Thermo Fisher Scientific, Inc. (Waltham, MA) and stored per manufacturer's instructions. Amikacin was suspended and diluted in Middlebrook 7H9 broth (BD Difco™, Franklin Lakes, NJ) with 0.2% glycerol and 10% albumin-dextrose-catalase (ADC, BD BBL™, Franklin Lakes, NJ) immediately prior to use. Azithromycin was suspended in dimethyl sulfoxide (Fisher Scientific International, Inc., Hampton, NH) then further diluted 1:1000 in 7H9 with 0.2% glycerol and 10% ADC immediately prior to use.

Bacterial stains and sources. M. abscessus 19977 (smooth morphotype) was originally isolated from an individual with a knee infection. M. avium 25291 was originally isolated from the infected liver of a chicken. Both isolates were procured from the American Type Culture Collection. M. abscessus clinical isolates 1, 2 and 3 (smooth morphotypes) were recovered from the sputum of people with cystic fibrosis (PwCF) s.

Isolation and Purification of Recombinant HupB. M. tuberculosis HupB was PCR amplified using the following oligonucleotides 5′-GCGTGCATATGAACAAAGCAGAGCT CATTGACGT-3′ (SEQ ID NO: 41) and 5′-CGTGGCTCTTCCGCACGCTTTGCGACCCC GCCGAG-3′ (SEQ ID NO: 42). Recombinant HupB was generated via previously described protocol (Novotny et al., 2016, EBioMedicine 10, 33-44), concentrated via centrifugal filter (3000 MWCO) and dialyzed against storage buffer (50 mmol/L Tris pH=7.4, 600 mmol/L KCl, 1 mmol/L EDTA, 10% glycerol) then stored at-80° C. until used. Approximately 200 ng of recombinant HupB was separated by SDS-PAGE using a 4-20% gradient gel at 5.6 V/cm for 1 hr. Expected molecular mass of HupB=28 kDa. Relative purity of HupB was determined by silver stain (Pierce™ Silver Stain Kit, Thermo Fisher Scientific, Inc., Waltham, MA)

Recognition of HupB by HuTipMab by ELISA. Purified recombinant HupB, tip-chimer peptide (a chimeric peptide which mimics protective epitopes of a DNABII protein and used to generate HuTipMab, positive control), and tail-chimer peptide (Novotny et al., 2016, EBioMedicine 10, 33-44) (a chimeric peptide that mimics non-protective epitopes of a DNABII protein, negative control) were suspended in phosphate buffered saline (PBS) (pH=7.4). One ug of each was added to wells of a Falcon® 96-well plate in duplicate and incubated for 1 hr at 37° C. Fluid was removed and wells were washed twice with PBS containing 1:2000 v/v Tween™-20 (PBS-T). Wells were blocked with 3% dry milk in PBS-T for 1 hr at 37° C. then washed twice with PBS-T. One set of the samples received 0.1 ug HuTipMab/well, whereas the other set received PBS-T alone (background control) after which all were incubated at 37° C. for 1 hr. Wells were washed 3 times with PBS-T, followed by the addition of goat anti-human IgG conjugated to horseradish peroxidase (1:5000 dilution) (Novus Biologicals LLC, Centennial, CO) in PBS-T incubated at 37° C. for 1 hr. Wells were washed 3 times with PBS-T and color was developed over 15 min at room temperature by addition of 1-Step™ Ultra TMB (Pierce™). Plates were read at 650 nm by FLUOstar Omega plate reader (BMG Labtech, Cary, NC) followed by visualization by Fluorchem M gel reader (ProteinSimple, Inc., Santa Clara, CA) with trans-UV light and 593 nm filter. Assays were repeated 3 times on separate days.

Biofilm formation by M. abscessus and M. avium. Stocks of M. abscessus or M. avium were maintained frozen in 7H9 broth containing 10% oleic acid-albumin-dextrose-catalase (OADC, Hardy Diagnostics, Santa Maria, CA), 30% glycerol and 0.05% Tween™ 80 at 2×108 CFU/mL and stored at −80° C. Stocks were gently thawed on ice, and bacteria pelleted by centrifugation at 21,100×g for 5 min at room temperature. Supernatants were discarded, pellet resuspended, and centrifuged again. Following centrifugation, bacteria were suspended to a final volume of 1 mL in 7H9 containing 10% OADC and 0.05% Tween™-80. Stocks were diluted ten-fold in 7H9/OADC/Tween™-80 to yield a final concentration of 2×10⁷ CFU/mL, and 200 μL was used to inoculate each well of an 8-well chambered coverglass slide (Cellvis, Mountainview, CA). Biofilms of M. abscessus or M. avium were allowed to form at 37° C. and 5% CO₂ in a humidified atmosphere until the biofilms grew to a height ˜30 μm as determined by confocal laser scanning microscopy (CLSM) and assessed by COMSTAT2. These incubation times were determined to be 72 hrs for M. abscessus versus 2 wks for slower growing M. avium. Biofilms of M. abscessus clinical isolates 1, 2 and 3 were also grown for an additional 24 hrs (96 hrs total) to evaluate whether HuTipMab could disrupt these even more mature biofilms.

Disruption of M. abscessus and M. avium biofilms by HuTipMab. Medium was aspirated from M. abscessus or M. avium biofilms, then they were gently washed twice with 200 μL equilibrated 1× Dulbecco's phosphate-buffered saline (DPBS) without calcium or magnesium (Corning, Corning, NY). Biofilms were then incubated with either 7H9 alone, 5 ug HuIgG, or with 5, 7.5 or 10 ug HuTipMab at 37° C. with 5% CO₂ in a humidified atmosphere for 30 min. To assay time-dependent disruption, additional wells were incubated with 5 ug HuTipMab for 60 min. After incubation, biofilms were gently washed once with 200 μL equilibrated DPBS.

Residual biofilm was stained with FM 1-43FX (Invitrogen) by incubation for 15 min statically in the dark. Stain was removed and biofilms were gently washed twice with DPBS then fixed for ≥3 hr (1.6% paraformaldehyde, 2.5% glutaraldehyde and 4% acetic acid in 0.1 M phosphate buffer). Fixative was removed and replaced with DPBS then biofilms were visualized and imaged with a ZEISS CLSM800 microscope. Images were analyzed by COMSTAT2 to calculate relative biomass values (μm3/μm2). Values represent the mean of 3 biological replicates. Percent disruption was calculated as [mean biomass of wells treated with HuIgG−mean biomass of wells treated with HuTipMab]/[mean biomass of wells treated with HuIgG])×100.

Antibiotic mediated killing of newly released M. abscessus or M. avium. M. abscessus or M. avium biofilms were incubated with medium alone (for recovery of those bacteria growing/residing planktonically in the fluids above the biofilm) or 5 ug HuTipMab (to generate NRel) both with and without antibiotics. The concentration of each antibiotic used was pre-determined to limit killing of planktonic bacteria to ˜25% to facilitate detection of any enhanced relative killing of NRel. For assay of relative killing of clinical M. abscessus isolates, Applicant elected to test isolates 1 and 3 as these showed greater antibiotic tolerance, requiring greater concentrations of amikacin and azithromycin to reach ˜25% killing than did the lab passaged isolates and thereby represented a more clinically relevant situation.

To determine relative susceptibility of planktonic NTM to amikacin, biofilms were prepared and gently washed twice with DPBS as described above, then treated with 200 μL of either: 7H9 (growth control); 7H9+0.5 μg amikacin/mL for M. abscessus 19977, +12 μg amikacin/mL for M. avium, +0.7 μg amikacin/mL for M. abscessus clinical isolate 1 or +lug amikacin/mL for M. abscessus clinical isolate 3. To determine relative sensitivity of NTM NRel, biofilms were treated with 200 μL 7H9+5 μg HuTipMab alone (NRel growth control) or 7H9+5 μg HuTipMab+respective amikacin concentrations used above. Treated biofilms were incubated statically at 37° C. in 5% CO₂ and humidity for 2 hr, after which 150 μL was collected from each well and dispensed into 1.5 mL Eppendorf microcentrifuge tubes, then pulse-vortexed with two sterile 3 mm glass beads (Fisher Scientific International, Inc., Hampton, NH) to disrupt aggregates. Suspensions were gently sonicated for 2 min in a waterbath sonicator (Ultrasonic Bath 2.8 L, Fisher Scientific) to further disrupt any aggregates. After sonication, samples were diluted and plated on Middlebrook 7H10 agar and incubated at 37° C. with 5% CO₂ in a humidified atmosphere to assess relative CFU.

To determine azithromycin-mediated killing, the same protocol as above was used with one adjustment. To avoid acidification of medium due to bacterial growth in wells to be treated with azithromycin which is unstable at lower pH (Johnson et al., 1999), these cultures were incubated for 2 hr at 37° C. without 5% CO₂. Wells were treated with 200 μL of either: 7H9 (growth control); 7H9+5 μg azithromycin/mL for M. abscessus 19977, +16 μg azithromycin/mL for M. avium, +10 μg azithromycin/mL for M. abscessus clinical isolate 1, or +8 μg azithromycin/mL for M. abscessus clinical isolate 3. 7H9+5 μg HuTipMab (NRel growth control) or 7H9+5 μg HuTipMab+respective azithromycin concentrations used above. Percent killing was then calculated as [growth control CFU/mL-NRel or planktonic CFU/mL]/[growth control CFU/mL].

Statistical analysis. Results are expressed as mean±SD of 3 biological replicates with 3 technical replicates each. Comparisons between groups were made with unpaired t-tests. All statistical analyses were performed with Graphpad (Prism) software V9.

### Example 2: Results

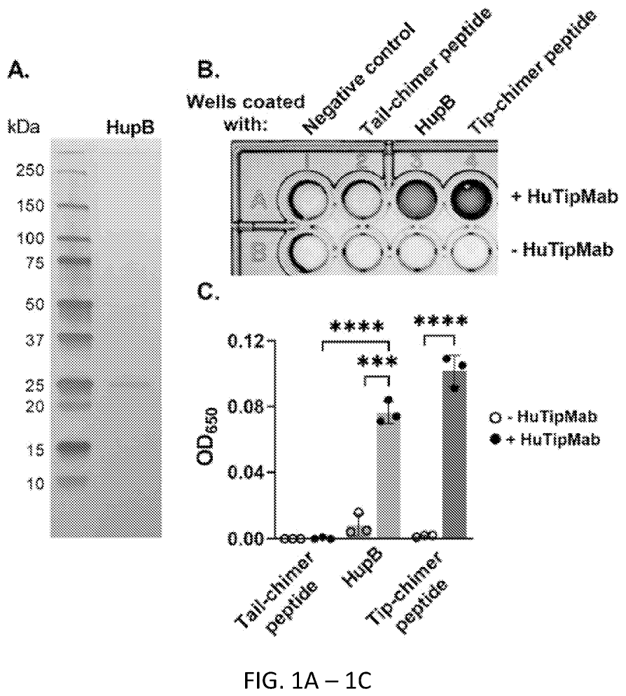

Validation of HuTipMab specificity Assessment. Purity of isolated recombinant HupB was confirmed via silver stain (FIG. 1A). Recognition of HupB by HuTipMab was shown via ELISA wherein HuTipMab recognized both HupB and the tip-chimer peptide; no color developed in the absence of HuTipMab (P<0.001-0.0001) (FIG. 1B & FIG. 1C). HuTipMab did not recognize the tail-chimer peptide.

Assessment of relative HuTipMab-induced NTM biofilm disruption by CLSM.

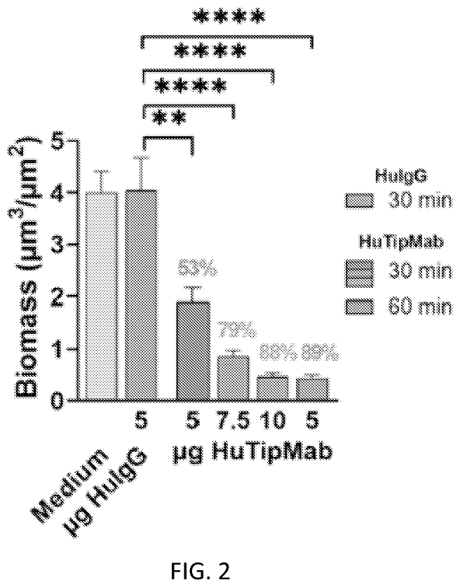

Applicant next determined HuTipMab's biofilm disruption capabilities by incubation of 72 hr M. abscessus 19977 biofilms or 2 wk M. avium biofilms with increasing concentrations of, or an increased incubation period with, HuTipMab. After incubation, newly released bacteria (“NRel”) were removed and the residual biofilms analyzed by Confocal Laser Scanning Microscope (CLSM). Image analysis revealed that HuTipMab disrupted M. abscessus 19977 biofilms significantly more than biofilms incubated with medium alone or with HuIgG (P<0.01-0.0001) (FIG. 2). Additionally, M. abscessus 19977 biofilms were disrupted in a dose- and time-dependent manner as 30 min incubation with 5, 7.5 or 10 μg HuTipMab resulted in 53%, 79% or 88% disruption respectively, whereas disruption was 89% when incubated with 5 μg HuTipMab for 60 min.

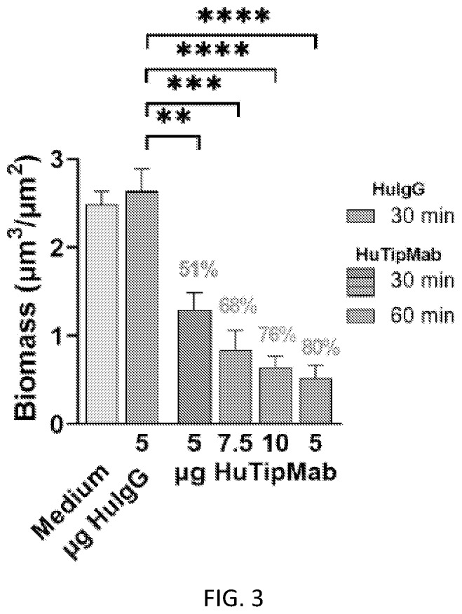

Similarly, M. avium biofilms were significantly disrupted by incubation with HuTipMab (P<0.01-0.0001) (FIG. 3). M. avium biofilms were also disrupted in a dose- and time-dependent manner wherein disruption by HuTipMab at 5, 7.5 or 10 μg for 30 min was 51%, 68% or 76%, respectively whereas when incubated with 5 μg HuTipMab for 60 min, disruption was 80%.

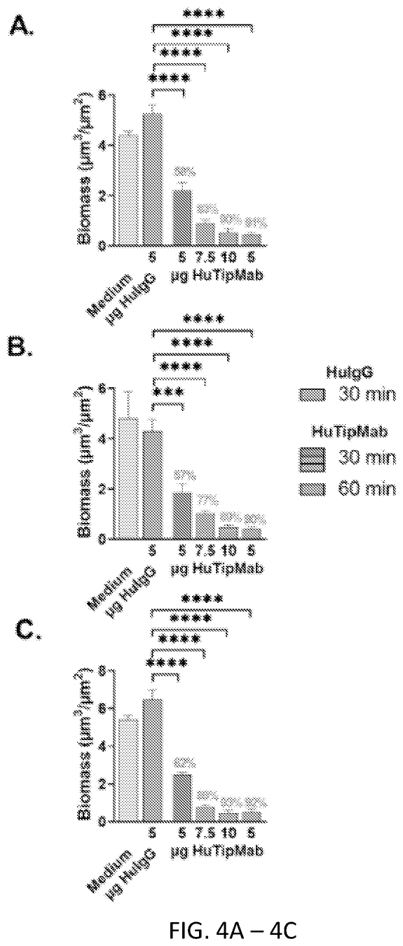

Significant dose- and time-dependent disruption of 72 hr biofilms formed by all 3 clinical isolates of M. abscessus was also evident (P<0.001-0.0001) (FIGS. 4A-4C). Disruption by HuTipMab at 5, 7.5 or 10 μg for 30 min was 57%-62%, 77%-88% or 89%-93%, respectively whereas when incubated with 5 μg HuTipMab for 60 min, disruption was 90%-92%. The more mature 96 hr biofilms formed by these 3 clinical isolates were similarly significantly disrupted by HuTipMab with relative mean percent disruption of 57%-59%, 76%-89% or 89%-94% when incubated with 5, 7.5 or 10 μg for 30 min, respectively and 90%-93% when incubated with 5 μg HuTipMab for 60 min (P<0.05-0.0001) (data not shown).

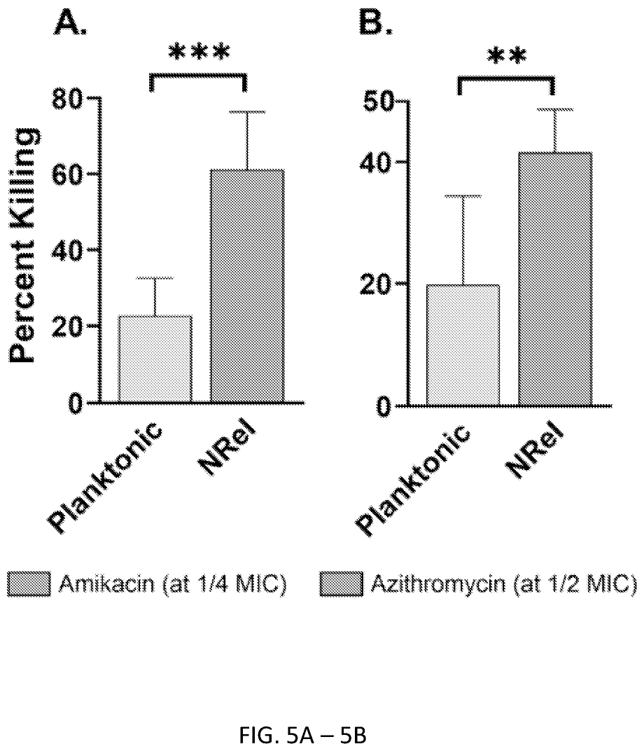

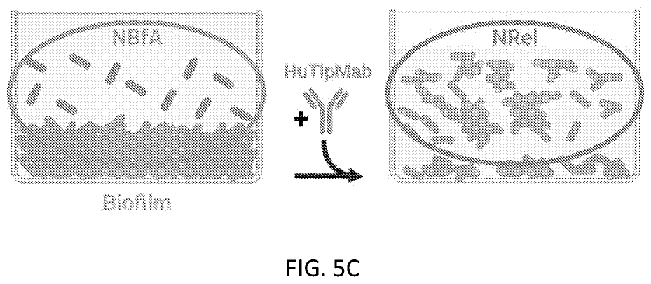

Enhanced killing of NTM NRel by antibiotics commonly used to treat NTM infections. To determine whether NTM NRel demonstrated heightened antibiotic susceptibility, Applicant assessed relative killing by amikacin and azithromycin. Killing of planktonic M. abscessus 19977 by amikacin or azithromycin was limited to 23% and 20%, respectively whereas M. abscessus 19977 NRel were significantly more sensitive to both antibiotics with killing at 61% and 42%, respectively (FIG. 5A & FIG. 5B) (P<0.001 or 0.01, respectively). Notably, this enhanced killing occurred when amikacin and azithromycin were used at ¼ and ½ the reported minimum inhibitory concentrations (MIC), respectively.

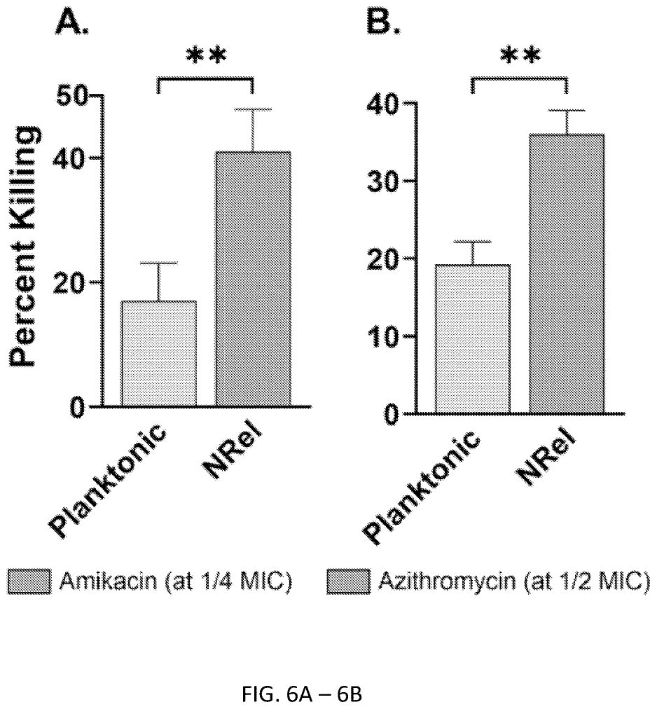

Similarly, M. avium NRel were significantly more susceptible to antibiotic killing than their isogenic planktonic counterpart with percent killing of planktonic M. avium limited to 17% and 19% by amikacin and azithromycin, respectively, whereas that for M. avium NRel was 41% and 36%, respectively (P<0.01) (FIG. 6A & FIG. 6B). This significantly enhanced susceptibility of M. avium NRel to killing by amikacin and azithromycin was observed when used at ¼ and ½ of their respective MICs.

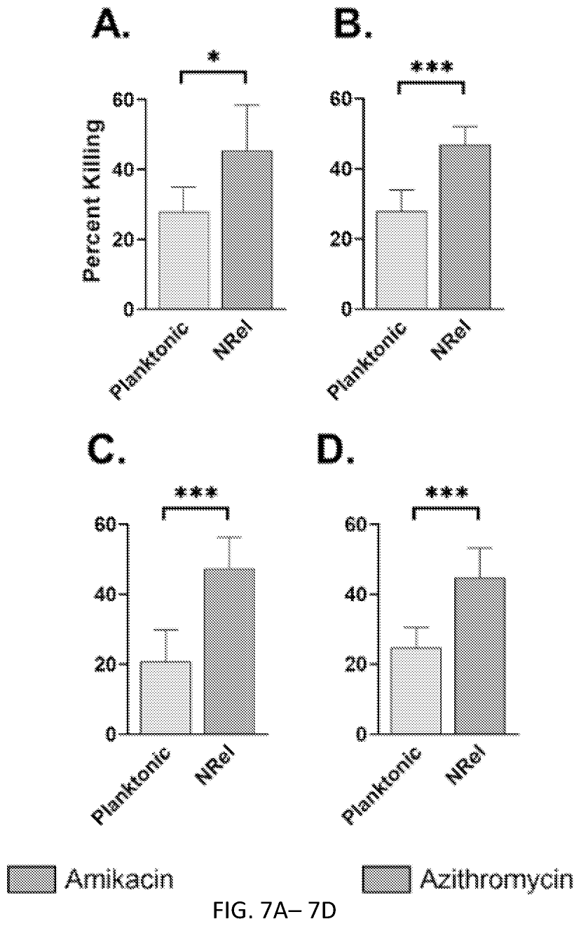

NRel from disrupted 72 hr biofilms formed by M. abscessus clinical isolates 1 or 3 were also significantly more susceptible to killing by amikacin and azithromycin than their isogenic planktonic counterparts (P<0.05-0.001) (FIGS. 7A-7D). Percent killing of planktonic M. abscessus was limited to 28% and 28% for clinical isolate 1 and 21% and 25% for clinical isolate 3 by amikacin and azithromycin, respectively, whereas killing of NRel was 45% and 47% for clinical isolate 1 or 47% and 45% by amikacin and azithromycin, respectively. Despite the greater concentrations of antibiotics required to achieve similar levels of killing as that for either planktonically grown clinical isolates or the lab-passaged M. abscessus 19977, as could be expected, note that greater sensitivity to killing of these anti-DNABII inducted NRel was achieved at the same concentration that killed only ˜25% of the corresponding planktonic populations.

Antibiotic therapy for PwCF who are culture positive for NTM involves a prolonged regimen with multiple antibiotics, the potential for multiple sequelae, and an unacceptably high rate of clinical failure. While modulator therapy both reduces symptoms and frequency of NTM-positive cultures, it remains to be determined if this latter outcome is due to reduced ability to collect sputum or a true decline in NTM prevalence. Additionally, modulator treatment remains inaccessible to a majority of PwCF due to both cost and availability that is currently limited to the US, Europe, Australia, and New Zealand. New strategies to enhance the effectiveness of existing antibiotic therapies are thus warranted, particularly given that there is now rather limited investment in new antibiotic discovery. Novel approaches including those that target the biofilm matrix to release resident bacteria for elimination by either antibiotics or host immune effectors are a high priority.

### Example 3

This experiment provides a porcine model for pre-clinical testing of antibodies and antigen-binding fragments thereof alone or in combination with the disclosed additional active agents to treat cystic fibrosis. See Stoltz et al. (2010) Science Translational Medicine 2 (29): 29-31. Cystic fibrosis is an autosomal recessive disease due to mutations in a gene that encodes the CF transmembrane conductance regulator (called CFTR) anion channel. In this model, pigs which have been specifically bred to carry a defect in the genes called “CFTR” and called CF pigs spontaneously develop hallmark features of CF lung disease that includes infection of the lower airway by multiple bacterial species. The pigs can receive the antibodies or antigen-binding fragments thereof alone or in combination with an antibiotic, in one aspect, administered to the lungs of these animals by nebulization to assess the amelioration of the signs of disease and associated pathologies.

### Example 4

Applicants also provide a pre-clinical model for tuberculosis (TB). See Ordway et al. (2010) Anti. Agents and Chemotherapy 54:1820. The microorganism Mycobacterium tuberculosis is responsible for a growing global epidemic. Current figures suggest that there are approximately 8 million new cases of TB and about 2.7 million deaths due to TB annually. In addition to the role of this microbe as a co-infection of individuals with HIV (of the ^{˜}45 million infected with HIV, estimates are that ^{˜}1/3 are also co-infected with M. tuberculosis), its particularly troublesome that isolates have become highly resistant to multiple drugs and no new drug for TB has been introduced in over a quarter of a century. In this animal model, SPF guinea pigs are maintained in a barrier colony and infected via aerosolized spray to deliver ^{˜}20 cfu of M. tuberculosis strain Erdman K01 bacilli into their lungs. Animals are sacrificed with determination of bacterial load and recovery of tissues for histopathological assessment on days 25, 50, 75, 100, 125 and 150 days post-challenge. Unlike mice which do not develop classic signs of TB, guinea pigs challenged in this manner develop well-organized granulomas with central necrosis, a hallmark of human disease. Further, like humans, guinea pigs develop severe pyogranulomatous and necrotizing lymphadenitis of the draining lymph nodes as part of the primary lesion complex. Use of this model provides a pre-clinical screen to confirm and identify therapeutic as well as preventative strategies for reduction and/or elimination of the resulting M. tuberculosis biofilms which have been observed to form in the lungs of these animals subsequent to challenge and are believed to contribute to both the pathogenesis and chronicity of the disease.

### Experimental Discussion

Applicant developed such a strategy using an epitope-targeted monoclonal antibody against the DNABII proteins. Although this approach has been shown to effectively disrupt biofilms formed by Gammaproteobacteria (Brockson et al., 2014, Mol Microbiol 93, 1246-1258; Freire et al., 2017, Mol Oral Microbiol 32, 74-88; Goodman et al., 2011, Mucosal Immunol 4, 625-637; Kurbatfinski et al., 2022, Antimicrob Agents Chemother., 66, e0187721; Kurbatfinski et al., 2023, Front Microbiol 14, 1202215), it was not clear if HuTipMab treatment would also be effective against NTM, which belong to the class Actinomycetia and are distinguished by a thick, mycolic acid-rich cell wall.

Comparative genomics showed that NTM can express a DNABII homologue and in silico analysis revealed that mycobacterial HUs have two domains. The N-terminal 106 amino acid (AA) domain has high similarity to other DNABII proteins (all are ˜90-105 AAs in length), whereas the 108 amino acid C-terminal domain has a eukaryotic H1 histone-like motif. Further, 104 out of 106 AAs within the translated sequence of the N-terminal DNABII-like domain are identical between the HU proteins expressed by M. tuberculosis and M. avium, whereas 99 out of 106 perfect matches for HU expressed by M. abscessus. All three DNABII homologues expressed by these mycobacterial species share at least 37 consecutive identical AAs, which comprise the tip region against which HuTipMab is targeted and which likely accounts for its ability to recognize HupB of M. tuberculosis by ELISA. Both M. abscessus and M. avium form biofilms that incorporate eDNA into the EPS matrix, however it was unknown whether these biofilms also incorporated DNABII proteins within the eDNA-rich biofilm matrix nor was it known whether HuTipMab, generated against specific protective domains of a traditional DNABII protein would both recognize the unique mycobacterial DNABII homologue and actively disrupt NTM biofilms. Applicant shows here that HuTipMab did indeed effectively disrupt biofilms formed by both M. abscessus and M. avium in a dose- and time-dependent manner which indicated that HuTipMab retained recognition and biofilm-disruptive capabilities against NTM, inclusive of biofilms formed by M. abscessus isolates recovered from PwCF, which likely better represent M. abscessus found in the disease site than lab-passaged M. abscessus 19977.

Once disrupted, NRel of M. abscessus 19977, M. avium, as well as that of M. abscessus clinical isolates 1 and 3, displayed increased susceptibility to two highly clinically relevant antibiotics. NTM NRel were significantly more sensitive to amikacin and azithromycin compared with their isogenic planktonic counterparts, two antibiotics which are ineffective when NTM are within biofilms (Clary et al., 2018, Antimicrob Agents Chemother., 62). Notably, enhanced antibiotic susceptibility of NRel occurred at fractions of the planktonic MIC for M. abscessus 19977 and M. avium, and similarly, NRel of both clinical isolates were more susceptible to antibiotic-mediated killing than their planktonic counterparts at the same concentration. This outcome is likely to have been aided by the fact that amikacin and azithromycin have greater access to their targets after release of NTM from their protective biofilms.

Applicant's data support a combinatorial therapeutic strategy for those infected with NTM, where biofilms contribute significantly to decline in lung function and poor quality of life. In one aspect, a method is provide for nebulizing HuTipMab into the lungs of these individuals to disrupt biofilm aggregates and release NTM from the antibiotic-tolerant biofilm into the NRel state such that co-delivered antibiotics could rapidly kill the induced NTM NRel. This strategy would empower existing antibiotics, improve clinical outcomes and also decrease the length of antibiotic treatment for PwCF, as well as the growing population of people without CF but who nonetheless have a recalcitrant NTM infections.

### EQUIVALENTS

It is to be understood that while the disclosure has been described in conjunction with the above embodiments, that the foregoing description and examples are intended to illustrate and not limit the scope of the disclosure. Other aspects, advantages and modifications within the scope of the disclosure will be apparent to those skilled in the art to which the disclosure pertains.

Unless otherwise defined, all technical and scientific terms used herein have the same meaning as commonly understood by one of ordinary skill in the art to which this disclosure belongs. All nucleotide sequences provided herein are presented in the 5′ to 3′ direction.

The embodiments illustratively described herein may suitably be practiced in the absence of any element or elements, limitation or limitations, not specifically disclosed herein. Thus, for example, the terms “comprising,” “including,” containing,” etc. shall be read expansively and without limitation. Additionally, the terms and expressions employed herein have been used as terms of description and not of limitation, and there is no intention in the use of such terms and expressions of excluding any equivalents of the features shown and described or portions thereof, but it is recognized that various modifications are possible within the scope of the disclosure.

Thus, it should be understood that although the present disclosure has been specifically disclosed by specific embodiments and optional features, modification, improvement and variation of the embodiments therein herein disclosed may be resorted to by those skilled in the art, and that such modifications, improvements and variations are considered to be within the scope of this disclosure. The materials, methods, and examples provided here are representative of particular embodiments, are exemplary, and are not intended as limitations on the scope of the disclosure.

The scoped of the disclosure has been described broadly and generically herein. Each of the narrower species and subgeneric groupings falling within the generic disclosure also form part of the disclosure. This includes the generic description with a proviso or negative limitation removing any subject matter from the genus, regardless of whether or not the excised material is specifically recited herein.

In addition, where features or aspects of the disclosure are described in terms of Markush groups, those skilled in the art will recognize that embodiments of the disclosure may also thereby be described in terms of any individual member or subgroup of members of the Markush group.

All publications, patent applications, patents, and other references mentioned herein are expressly incorporated by reference in their entirety, to the same extent as if each were incorporated by reference individually. In case of conflict, the present specification, including definitions, will control.

### Non-Limiting Embodiments of the Disclosure

Embodiment 1. A method for preventing or treating an infection caused by a Nontuberculous mycobacterium (NTM) species in a subject comprising administering to the subject an effective amount of an antibody or an antigen-binding fragment thereof that binds to a tip region of a DNABII peptide.

Embodiment 2. A method for sensitizing a biofilm to an antibiotic agent or a method for disrupting a biofilm, wherein the biofilm comprises a Nontuberculous mycobacterium (NTM) species, the method comprising contacting the biofilm with an antibody or an antigen-binding fragment thereof that binds to a tip region of a DNABII peptide.

Embodiment 3. The method of embodiment 1 or embodiment 2, wherein the antibody or the antigen-binding fragment thereof comprises one or more of:
a. a heavy chain (HC) immunoglobulin variable domain sequence comprising a sequence of amino acid (aa) 25 to aa 144 of SEQ ID NO: 21 or an equivalent thereof, and a light chain (LC) immunoglobulin variable domain sequence comprising a sequence of aa 21 to aa 132 of SEQ ID NO: 22 or an equivalent thereof;
b. a heavy chain (HC) immunoglobulin variable domain sequence comprising a sequence of aa 25 to aa 144 of SEQ ID NO: 24 or an equivalent thereof, and a light chain (LC) immunoglobulin variable domain sequence comprising a sequence of aa 21 to aa 132 of SEQ ID NO: 25 or an equivalent thereof;
c. a heavy chain complementarity-determining region 1 (CDRH1) comprising a sequence of GFTFRTY (SEQ ID NO: 58) (aa 50 to aa 56 of SEQ ID NO: 9 or 10 or 11 or 24), a heavy chain complementarity-determining region 2 (CDRH2) comprising a sequence of GSDRRH (SEQ ID NO: 59) (aa 76 to aa 81 of SEQ ID NO: 9 or 10 or 11 or 24), a heavy chain complementarity-determining region 3 (CDRH3) comprising a sequence of VGPYDGYYGEFDY (SEQ ID NO: 60) (aa 121 to aa 133 of SEQ ID NO: 9 or 10 or 11 or 24), a light chain complementarity-determining region 1 (CDRL1) comprising a sequence of QSLLDSDGKTF (SEQ ID NO: 61) (aa 47 to aa 57 of SEQ ID NO: 15 or 16 or 17 or 25), a light chain complementarity-determining region 2 (CDRL2) comprising a sequence of LVS (aa 75 to aa 77 of SEQ ID NO: 15 or 16 or 17 or 25), and a light chain complementarity-determining region 3 (CDRL3) comprising a sequence of WQGTHFP (SEQ ID NO: 62) (aa 114 to aa 120 of SEQ ID NO: 15 or 16 or 17 or 25);
d. a heavy chain complementarity-determining region 1 (CDRH1) comprising, or consisting essentially of, or yet further consisting of a sequence of GFTFSRYG (SEQ ID NO: 63) (aa 50 to aa 57 of SEQ ID NO: 12 or 13 or 14), a heavy chain complementarity-determining region 2 (CDRH2) comprising, or consisting essentially of, or yet further consisting of a sequence of ISSGGSYT (SEQ ID NO: 64) (aa 75 to aa 82 of SEQ ID NO: 12 or 13 or 14), a heavy chain complementarity-determining region 3 (CDRH3) comprising, or consisting essentially of, or yet further consisting of a sequence of ERHGGDGYWYFDV (SEQ ID NO: 65) (aa 121 to aa 133 of SEQ ID NO: 12 or 13 or 14), a light chain complementarity-determining region 1 (CDRL1) comprising, or consisting essentially of, or yet further consisting of a sequence of QSLLDSDGKTF (SEQ ID NO: 61) (aa 47 to aa 57 of SEQ ID NO: 15 or 16 or 17 or 25), a light chain complementarity-determining region 2 (CDRL2) comprising, or consisting essentially of, or yet further consisting of a sequence of LVS (aa 75 to aa 77 of SEQ ID NO: 15 or 16 or 17 or 25), and a light chain complementarity-determining region 3 (CDRL3) comprising, or consisting essentially of, or yet further consisting of a sequence of WQGTHFPYT (SEQ ID NO: 66) (aa 114 to aa 122 of SEQ ID NO: 15 or 16 or 17 or 25);
and/or
e. a heavy chain (HC) immunoglobulin variable domain comprising the amino acid sequence of amino acids 25 to 144 of SEQ ID NO: 9; and a light chain (LC) immunoglobulin variable domain comprising the amino acid sequence of amino acids 21 to 132 of SEQ ID NO: 15, SEQ ID NO: 16, or SEQ ID NO: 17.

Embodiment 4. The method of any one of embodiments 1-3, wherein the antibody or the antigen-binding fragment thereof further comprises a constant region selected from the group of: an IgA constant region, an IgD constant region, an IgE constant region, an IgG constant region or an IgM constant region.

Embodiment 5. The method of embodiment 4, wherein the constant region is an IgG1 constant region.

Embodiment 6. The method of any one of embodiments 1-5, wherein the antigen-binding fragment is selected from the group of Fab, F(ab′) 2, Fab′, scFv, or Fv.

Embodiment 7. The method of any one of embodiments 3-6, wherein the equivalent to an amino acid sequence comprises a polypeptide having at least 80% amino acid identity to the amino acid, or wherein an equivalent to the amino acid sequence comprises a polypeptide that is encoded by a polynucleotide that hybridizes under conditions of high stringency to the complement of the polynucleotide encoding the amino acid sequence.

Embodiment 8. The method of any one of embodiments 1-7, wherein the antibody or the antigen-binding fragment thereof further comprises a modification.

Embodiment 9. The method of embodiment 8, wherein the modification is selected from the group of PEGylation, a PEG mimetic, polysialyation, HESylation or glycosylation.

Embodiment 10. The method any one of embodiments 2-9, wherein the antibiotic agent comprises amikacin, azithromycin, dactinomycin, bleomycin, daunorubicin, doxorubicin, idarubicin, mithramycin, mitomycin, mitoxantrone, plicamycin, methicillin, vancomycin, daptomycin, mupirocin, penicillin, cloxacillin, erythromycin, clarithromycin, roxithromycin, telithromycin, spiramycin, fidaxomicin, rifampin, ethambutol, streptomycin or anthramycin, that optionally is administered in an amount that from about 25% to about 50% of the MIC of the antibiotic. In a further embodiment the antibody or antigen-binding fragment thereof and the antibiotic are administered concurrently or subsequently to each other, e.g., the antibiotic subsequent to, or alternatively prior to the antibody or the antigen-binding fragment thereof.

Embodiment 11. The method any one of embodiments 2-10, further comprising contacting the biofilm with an effective amount of a synthetic or recombinant polypeptide comprising, or consisting essentially of, or yet further consisting of mB Box-97 that consists of the amino acids 90 to 176 from the coding sequence of the native human HMGB1 protein shown as SEQ ID NO: 45 with a cysteine to serine point mutation at amino acid 106 or an equivalent thereof. The contacting can be concurrently or sequentially.

Embodiment 12. The method any one of embodiments 2-10, further comprising contacting the biofilm with an effective amount of a synthetic or recombinant polypeptide comprising, or consisting essentially of, or yet further consisting of mB Box-97 that consists of the amino acids 80 to 176 from the coding sequence of the native human HMGB1 protein shown as SEQ ID NO: 45 with a cysteine to serine point mutation at amino acid 106 or an equivalent thereof.

Embodiment 13. The method of embodiment 11 or embodiment 12, wherein the synthetic or recombinant polypeptide consists of SEQ ID NO: 48.

Embodiment 14. The method any one of embodiments 2-13, further comprising contacting the biofilm with an effective amount of a DNA-binding agent.

Embodiment 15. The method of embodiment 14, wherein the DNA-binding agent comprises a Histone-like Nucleoid Structuring protein (H-NS), a polyamine, or a polycation.

Embodiment 16. The method of embodiment 15, wherein the H-NS comprises an amino acid sequence having at least 60% (e.g., at least 60, 65, 70, 75, 80, 85, 90, 95, 99% or more) identity to an amino acid sequence selected from SEQ ID NOs: 51-56.

Embodiment 17. The method any one of embodiments 1 and 3-10, further comprising administering to the subject an effective amount of an at least one antibiotic agent selected from amikacin, azithromycin, dactinomycin, bleomycin, daunorubicin, doxorubicin, idarubicin, mithramycin, mitomycin, mitoxantrone, plicamycin, methicillin, vancomycin, daptomycin, mupirocin, penicillin, cloxacillin, erythromycin, clarithromycin, roxithromycin, telithromycin, spiramycin, fidaxomicin, rifampin, ethambutol, streptomycin or anthramycin, that optionally is administered in an amount that from about 25% to about 50% of the MIC of the antibiotic. In a further embodiment the antibody or antigen-binding fragment thereof and the antibiotic are administered concurrently or subsequently to each other, e.g., the antibiotic subsequent to, or alternatively prior to the antibody or the antigen-binding fragment thereof.

Embodiment 18. The method any one of embodiments 1, 3-10 and 17, further comprising administering to the subject an effective amount of a synthetic or recombinant polypeptide comprising, or consisting essentially of, or yet further consisting of mB Box-97 that consists of the amino acids 90 to 176 from the coding sequence of the native human HMGB1 protein shown as SEQ ID NO: 45 with a cysteine to serine point mutation at amino acid 106 or an equivalent thereof.

Embodiment 19. The method any one of embodiments 1, 3-10 and 17, further comprising administering to the subject an effective amount of a synthetic or recombinant polypeptide comprising, or consisting essentially of, or yet further consisting of mB Box-97 that consists of the amino acids 80 to 176 from the coding sequence of the native human HMGB1 protein shown as SEQ ID NO: 45 with a cysteine to serine point mutation at amino acid 106 or an equivalent thereof.

Embodiment 20. The method of embodiment 18 or embodiment 19, wherein the synthetic or recombinant polypeptide consists of SEQ ID NO: 48.

Embodiment 21. The method any one of embodiments 1, 3-10 and 17-20, further comprising contacting the biofilm with an effective amount of a DNA-binding agent.

Embodiment 22. The method of embodiment 21, wherein the DNA-binding agent comprises a Histone-like Nucleoid Structuring protein (H-NS), a polyamine, or a polycation.

Embodiment 23. The method of embodiment 22, wherein the H-NS comprises an amino acid sequence having at least 60% (e.g., at least 60, 65, 70, 75, 80, 85, 90, 95, 99% or more) identity to an amino acid sequence selected from SEQ ID NOs: 51-56.

Embodiment 24. The method of any one of embodiments 1-23, wherein the NTM species is M. abscessus (Mab), M. avium (Mav), M. intracellulare, or M. chimaera.

Embodiment 25. The method of any of embodiments 1, 3-10 and 17, wherein the administration comprises one or more of: inhalation, oral administration, nasal administration, injection and/or topical application.

Embodiment 26. The method of any one of embodiments 1, and 3-10, and 15-25, wherein the subject is suffering from chronic obstructive pulmonary disease (COPD), primary ciliary dyskinesia, or cystic fibrosis (CF).

Embodiment 27. The method any one of embodiments 2-16 and 24, wherein the contacting is or.

| SEQUENCE LISTING | |
|---|---|
| SEQ ID NO: 1, human, IhfA, A tip fragment | |
| NFELRDKSSRPGRNPKTGDVV | |
| SEQ ID NO: 2, human IhfB, B tip fragment | |
| SLHHRQPRLGRNPKTGDSVNL | |
| SEQ ID NO: 3 (tip-chimeric peptide IhfA5-mIhfB4NTHI) | |
| RPGRNPX₁TGDVVPVSARRVV-X-FSLHHRQPRLGRNPX₁TGDSV | |
| wherein “X” is an optional amino acid linker sequence, | |
| optionally comprising, or consisting essentially of, | |
| or yet further consisting of between 1 to 20 amino acids | |
| wherein “X₁” is any amino acid or alternatively “X₁” is | |
| selected from the amino acids Q, R, K, S, or T. | |
| SEQ ID NO: 4 (tip-chimeric peptide IhfA5-mIhfB4NTHI) | |
| RPGRNPKTGDVVPVSARRVV-X-FSLHHRQPRLGRNPKTGDSV | |
| wherein “X” is an optional amino acid linker sequence | |
| optionally comprising between 1 to 20 amino acids. | |
| SEQ ID NO: 5 (tip-chimeric peptide IhfA5-mIhfB4NTHI) | |
| RPGRNPKTGDVVPVSARRVVGPSLFSLHHRQPRLGRNPKTGDSV | |
| SEQ ID NO. 6: Non-limiting exemplary linker: | |
| GGSGGS | |
| SEQ ID NO. 7: Non-limiting exemplary linker: | |
| GPSLKL. | |
| Non-limiting exemplary linker: | |
| GGG. | |
| SEQ ID NO: 9 (H10210 (1F8.F1 Humanized HC1)) | |
| Bold font indicates an exemplified variable region while the | |
| bold, italic and underlined font indicates exemplified CDRs. | |
| MDPKGSLSWRILLFLSLAFELSYGEVKLVESGGGLVQPGGSLRLSCAASGFTFRTY | |
| AMSWVRQAPGKGLEWVATIGSDRRHTYYPDSVKGRFTISRDNAKNTLYLQMNS | |
| LRAEDTAVYYCVGPYDGYYGEFDYWGQGTLVTVSSASTKGPSVFPLAPSSKSTSGG | |
| TAALGCLVKDYFPEPVTVSWNSGALTSGVHTFPAVLQSSGLYSLSSVVTVPSSSLGT | |
| QTYICNVNHKPSNTKVDKKVEPKSCDKTHTCPPCPAPELLGGPSVFLFPPKPKDTLMI | |
| SRTPEVTCVVVDVSHEDPEVKFNWYVDGVEVHNAKTKPREEQYNSTYRVVSVLTV | |
| LHQDWLNGKEYKCKVSNKALPAPIEKTISKAKGQPREPQVYTLPPSREEMTKNQVSL | |
| TCLVKGFYPSDIAVEWESNGQPENNYKTTPPVLDSDGSFFLYSKLTVDKSRWQQGN | |
| VFSCSVMHEALHNHYTQKSLSLSPG** | |
| SEQ ID NO: 10 (H10211 (1F8.F1 Humanized HC2)) | |
| Bold font indicates an exemplified variable region while the | |
| bold, italic and underlined font indicates exemplified CDRs. | |
| MDPKGSLSWRILLFLSLAFELSYGEVQLVESGGGLVQPGGSLRLSCAASGFTFRTY | |
| AMSWVRQAPGKGLEWVATIGSDRRHTYYPDSVKGRFTISRDNSKNTLYLQMNS | |
| LRAEDTAVYYCVGPYDGYYGEFDYWGQGTLVTVSSASTKGPSVFPLAPSSKSTSGG | |
| TAALGCLVKDYFPEPVTVSWNSGALTSGVHTFPAVLQSSGLYSLSSVVTVPSSSLGT | |
| QTYICNVNHKPSNTKVDKKVEPKSCDKTHTCPPCPAPELLGGPSVFLFPPKPKDTLMI | |
| SRTPEVTCVVVDVSHEDPEVKFNWYVDGVEVHNAKTKPREEQYNSTYRVVSVLTV | |
| LHQDWLNGKEYKCKVSNKALPAPIEKTISKAKGQPREPQVYTLPPSREEMTKNQVSL | |
| TCLVKGFYPSDIAVEWESNGQPENNYKTTPPVLDSDGSFFLYSKLTVDKSRWQQGN | |
| VFSCSVMHEALHNHYTQKSLSLSPG** | |
| SEQ ID NO: 11 (H10212 (1F8.F1 Humanized HC3)) | |
| Bold font indicates an exemplified variable region while the | |
| bold, italic and underlined font indicates exemplified CDRs. | |
| MDPKGSLSWRILLFLSLAFELSYGEVKLVQSGAEVKKPGASVKVSCKASGFTFRTY | |
| AMSWVRQAPGQRLEWVATIGSDRRHTYYPDKFQGRVTITRDNAKNTLYMELSS | |
| LRSEDTAVYYCVGPYDGYYGEFDYWGQGTLVTVSSASTKGPSVFPLAPSSKSTSGG | |
| TAALGCLVKDYFPEPVTVSWNSGALTSGVHTFPAVLQSSGLYSLSSVVTVPSSSLGT | |
| QTYICNVNHKPSNTKVDKKVEPKSCDKTHTCPPCPAPELLGGPSVFLFPPKPKDTLMI | |
| SRTPEVTCVVVDVSHEDPEVKFNWYVDGVEVHNAKTKPREEQYNSTYRVVSVLTV | |
| LHQDWLNGKEYKCKVSNKALPAPIEKTISKAKGQPREPQVYTLPPSREEMTKNQVSL | |
| TCLVKGFYPSDIAVEWESNGQPENNYKTTPPVLDSDGSFFLYSKLTVDKSRWQQGN | |
| VFSCSVMHEALHNHYTQKSLSLSPG** | |
| SEQ ID NO: 12 (H10213 (11E7.C7 Humanized HC1)) | |
| Bold font indicates an exemplified variable region while the | |
| bold, italic and underlined font indicates exemplified CDRs. | |
| MDPKGSLSWRILLFLSLAFELSYGEVQLVESGGGLVKPGGSLRLSCAASGFTFSRY | |
| GMSWVRQAPGKGLEWVATISSGGSYTYYTDSVKGRFTISRDNAKNSLYLQMNSL | |
| RAEDTAVYYCERHGGDGYWYFDVWGQGTMVTVSSASTKGPSVFPLAPSSKSTSG | |
| GTAALGCLVKDYFPEPVTVSWNSGALTSGVHTFPAVLQSSGLYSLSSVVTVPSSSLG | |
| TQTYICNVNHKPSNTKVDKKVEPKSCDKTHTCPPCPAPELLGGPSVFLFPPKPKDTL | |
| MISRTPEVTCVVVDVSHEDPEVKFNWYVDGVEVHNAKTKPREEQYNSTYRVVSVLT | |
| VLHQDWLNGKEYKCKVSNKALPAPIEKTISKAKGQPREPQVYTLPPSREEMTKNQV | |
| SLTCLVKGFYPSDIAVEWESNGQPENNYKTTPPVLDSDGSFFLYSKLTVDKSRWQQG | |
| NVFSCSVMHEALHNHYTQKSLSLSPG** | |
| SEQ ID NO: 13 (H10214 (11E7.C7 Humanized HC2)) | |
| Bold font indicates an exemplified variable region while the | |
| bold, italic and underlined font indicates exemplified CDRs. | |
| MDPKGSLSWRILLFLSLAFELSYGEVQLVESGGGLVKPGGSLRLSCAASGFTFSRY | |
| GMSWVRQAPGKGLEWVSTISSGGSYTYYTDSVKGRFTISRDNAKNSLYLQMNSL | |
| RAEDTAVYYCERHGGDGYWYFDVWGQGTMVTVSSASTKGPSVFPLAPSSKSTSG | |
| GTAALGCLVKDYFPEPVTVSWNSGALTSGVHTFPAVLQSSGLYSLSSVVTVPSSSLG | |
| TQTYICNVNHKPSNTKVDKKVEPKSCDKTHTCPPCPAPELLGGPSVFLFPPKPKDTL | |
| MISRTPEVTCVVVDVSHEDPEVKFNWYVDGVEVHNAKTKPREEQYNSTYRVVSVLT | |
| VLHQDWLNGKEYKCKVSNKALPAPIEKTISKAKGQPREPQVYTLPPSREEMTKNQV | |
| SLTCLVKGFYPSDIAVEWESNGQPENNYKTTPPVLDSDGSFFLYSKLTVDKSRWQQG | |
| NVFSCSVMHEALHNHYTQKSLSLSPG** | |
| SEQ ID NO: 14 (H10215 (11E7.C7 Humanized HC3)) | |
| Bold font indicates an exemplified variable region while the | |
| bold, italic and underlined font indicates exemplified CDRs. | |
| MDPKGSLSWRILLFLSLAFELSYGEVQLVESGGGLVQPGRSLRLSCTASGFTFSRY | |
| GMSWVRQAPGKGLEWVATISSGGSYTYYTDSVKGRFTISRDNAKNILYLQMNSL | |
| KTEDTAVYYCERHGGDGYWYFDVWGQGTMVTVSSASTKGPSVFPLAPSSKSTSG | |
| GTAALGCLVKDYFPEPVTVSWNSGALTSGVHTFPAVLQSSGLYSLSSVVTVPSSSLG | |
| TQTYICNVNHKPSNTKVDKKVEPKSCDKTHTCPPCPAPELLGGPSVFLFPPKPKDTL | |
| MISRTPEVTCVVVDVSHEDPEVKFNWYVDGVEVHNAKTKPREEQYNSTYRVVSVLT | |
| VLHQDWLNGKEYKCKVSNKALPAPIEKTISKAKGQPREPQVYTLPPSREEMTKNQV | |
| SLTCLVKGFYPSDIAVEWESNGQPENNYKTTPPVLDSDGSFFLYSKLTVDKSRWQQG | |
| NVFSCSVMHEALHNHYTQKSLSLSPG** | |
| SEQ ID NO: 15 (L10210 (1F8.F1 Humanized LC1)) | |
| Bold font indicates an exemplified variable region while the | |
| bold, italic and underlined font indicates exemplified CDRs. | |
| METDTLLLWVLLLWVPGSTGDVVMTQSPLSLPVTLGQPASISCRSSQSLLDSDGKT | |
| FLNWLQQRPGQSPRRLIYLVSKLDSGVPDRFSGSGSGTDFTLKISRVEAEDVGVY | |
| YCWQGTHFPYTFGQGTKLEIKRTVAAPSVFIFPPSDEQLKSGTASVVCLLNNFYPRE | |
| AKVQWKVDNALQSGNSQESVTEQDSKDSTYSLSSTLTLSKADYEKHKVYACEVTH | |
| QGLSSPVTKSFNRGEC* | |
| SEQ ID NO: 16 (L10211 (1F8.F1 Humanized LC2)) | |
| Bold font indicates an exemplified variable region while the | |
| bold, italic and underlined font indicates exemplified CDRs. | |
| METDTLLLWVLLLWVPGSTGDVVMTQSPLSLPVTLGQPASISCRSSQSLLDSDGKT | |
| FLNWLQQRPGQSPRRLIYLVSKRDSGVPDRFSGSGSGTDFTLKISRVEAEDVGVY | |
| YCWQGTHFPYTFGQGTKLEIKRTVAAPSVFIFPPSDEQLKSGTASVVCLLNNFYPRE | |
| AKVQWKVDNALQSGNSQESVTEQDSKDSTYSLSSTLTLSKADYEKHKVYACEVTH | |
| QGLSSPVTKSFNRGEC* | |
| SEQ ID NO: 17 (L10212 (1F8.F1 Humanized LC3)) | |
| Bold font indicates an exemplified variable region while the | |
| bold, italic and underlined font indicates exemplified CDRs. | |
| METDTLLLWVLLLWVPGSTGDVVMTQSPDSLAVSLGERATINCKSSQSLLDSDGKTF | |
| LNWLQKKPGQPPKRLIYLVSKLDSGVPDRFSGS | |
| GSGTDFTLTISSLQAEDVAVYYCWQGTHFPYTFGQGTKLEIK | |
| RTVAAPSVFIFPPSDEQLKSGTASVVCLLNNFYPREAKVQWKVDNALQSGNSQESVTE | |
| QDSKDSTYSLSSTLTLSKADYEKHKVYACEVTHQGLSSPVTKSFNRGEC* | |
| SEQ ID NO: 18 (L10213 (11E7.C7 Humanized LC1)) | |
| Bold font indicates an exemplified variable region while the | |
| bold, italic and underlined font indicates exemplified CDRs. | |
| METDTLLLWVLLLWVPGSTGDIQMTQSPSSLSASVGDRVTITCRASQDISNYLNW | |
| YQQKPGKAVKLLIYYTSRLHSGVPSRFSGSGSGTDYTLTISSLQPEDFATYFCQQ | |
| GNPLRTFGGGTKVEIKRTVAAPSVFIFPPSDEQLKSGTASVVCLLNNFYPREAKVQW | |
| KVDNALQSGNSQESVTEQDSKDSTYSLSSTLTLSKADYEKHKVYACEVTHQGLSSPV | |
| TKSFNRGEC* | |
| SEQ ID NO: 19 (L10214 (11E7.C7 Humanized LC2)) | |
| Bold font indicates an exemplified variable region while the | |
| bold, italic and underlined font indicates exemplified CDRs. | |
| METDTLLLWVLLLWVPGSTGDIQMTQSPSSLSASVGDRVTITCRASQDISNYLNW | |
| YQQKPGKAVKLLIYYTSRLHSGVPSRFSGSGSGTDYTLTISSLQPEDFATYYCQQ | |
| GNPLRTFGGGTKVEIKRTVAAPSVFIFPPSDEQLKSGTASVVCLLNNFYPREAKVQW | |
| KVDNALQSGNSQESVTEQDSKDSTYSLSSTLTLSKADYEKHKVYACEVTHQGLSSPV | |
| TKSFNRGEC* | |
| SEQ ID NO: 20 (L10215 (11E7.C7 Humanized LC3)) | |
| Bold font indicates an exemplified variable region while the | |
| bold, italic and underlined font indicates exemplified CDRs. | |
| METDTLLLWVLLLWVPGSTGDIVMTQSPATLSLSPGERATLSCRASQDISNYLNW | |
| YQQKPGQAVRLLIYYTSRLHSGIPARFSGSGSGTDYTLTISSLEPEDFAVYFCQQG | |
| NPLRTFGGGTKVEIKRTVAAPSVFIFPPSDEQLKSGTASVVCLLNNFYPREAKVQWK | |
| VDNALQSGNSQESVTEQDSKDSTYSLSSTLTLSKADYEKHKVYACEVTHQGLSSPVT | |
| KSFNRGEC* | |
| SEQ ID NO: 21 (Heavy Chain Consensus Sequence) | |
| MDPKGSLSWR ILLFLSLAFE LSYGEVqLVe SGgglvXPGg SlrlSCaASG | 50 |
| FTFXXYXMSW VRQAPGkgLE WVaTIXSXXX XTYYXDsvkG RfTIsRDNaK | 100 |
| NtLYlqmnSL raEDTAVYYC XXXXXXXYXX FDXWGQGTXV TVSSASTKGP | 150 |
| SVFPLAPSSK STSGGTAALG CLVKDYFPEP VTVSWNSGAL TSGVHTFPAV | 200 |
| LQSSGLYSLS SVVTVPSSSL GTQTYICNVN HKPSNTKVDK KVEPKSCDKT | 250 |
| HTCPPCPAPE LLGGPSVFLF PPKPKDTLMI SRTPEVTCVV VDVSHEDPEV | 300 |
| KFNWYVDGVE VHNAKTKPRE EQYNSTYRVV SVLTVLHQDW LNGKEYKCKV | 350 |
| SNKALPAPIE KTISKAKGQP REPQVYTLPP SREEMTKNQV SLTCLVKGFY | 400 |
| PSDIAVEWES NGQPENNYKT TPPVLDSDGS FFLYSKLTVD KSRWQQGNVF | 450 |
| SCSVMHEALH NHYTQKSLSL SPG** | 475 |
| Wherein X and a small letter can be substituted with any | |
| amino acid or alternatively, with an amino acid selected | |
| from SEQ ID NOs: 9-14, in the corresponding position. In | |
| one embodiment, X may also indicate absence of an amino | |
| acid residue. | |
| SEQ ID NO: 22 (Light Chain Consensus Sequence) | |
| METDTLLLWV LLLWVPGSTG DXvMTQSPXs LsvslGXrat isCrXSQXXX | 50 |
| XXXXXXXLNW XQQkPGqaXX XLIYXXSXIX SGvPdRFSGS GSGTDXTLtI | 100 |
| SslXXEDXav YyCXQGXXXX XTFGXGTKXE IKRTVAAPSV FIFPPSDEQL | 150 |
| KSGTASVVCL LNNFYPREAK VQWKVDNALQ SGNSQESVTE QDSKDSTYSL | 200 |
| SSTLTLSKAD YEKHKVYACE VTHQGLSSPV TKSFNRGEC* | 240 |
| Wherein X and a small letter can be substituted with any | |
| amino acid or alternatively, with an amino acid selected | |
| from SEQ ID NOs: 15-20, in the corresponding position. In | |
| one embodiment, X may also indicate absence of an amino | |
| acid residue. | |
| SEQ ID NO: 23 Human IgG1 constant region, Uniprot: P01857 | |
| ASTKGPSVFPLAPSSKSTSGGTAALGCLVKDYFPEPVTVSWNSGALTSGVHTFPAVL | |
| QSSGLYSLSSVVTVPSSSLGTQTYICNVNHKPSNTKVDKKVEPKSCDKTHTCPPCPAP | |
| ELLGGPSVFLFPPKPKDTLMISRTPEVTCVVVDVSHEDPEVKFNWYVDGVEVHNAK | |
| TKPREEQYNSTYRVVSVLTVLHQDWLNGKEYKCKVSNKALPAPIEKTISKAKGQPR | |
| EPQVYTLPPSRDELTKNQVSLTCLVKGFYPSDIAVEWESNGQPENNYKTTPPVLDSD | |
| GSFFLYSKLTVDKSRWQQGNVFSCSVMHEALHNHYTQKSLSLSPGK | |
| SEQ ID NO: 24 (Tip Heavy Chain Consensus Sequence) | |
| MDPKGSLSWR ILLFLSLAFE LSYGEVkLVe SGgglvqPGg SlrlSCaASG | 50 |
| FTFRTYAMSW VRQAPGkgLE WVATIGSDRR HTYYPDsvkG RfTIsRDNaK | 100 |
| NTLYlqmnSL RaEDTAVYYC VGPYDGYYGE FDYWGQGTLV TVSSASTKGP | 150 |
| SVFPLAPSSK STSGGTAALG CLVKDYFPEP VTVSWNSGAL TSGVHTFPAV | 200 |
| LQSSGLYSLS SVVTVPSSSL GTQTYICNVN HKPSNTKVDK KVEPKSCDKT | 250 |
| HTCPPCPAPE LLGGPSVFLF PPKPKDTLMI SRTPEVTCVV VDVSHEDPEV | 300 |
| KFNWYVDGVE VHNAKTKPRE EQYNSTYRVV SVLTVLHQDW LNGKEYKCKV | 350 |
| SNKALPAPIE KTISKAKGQP REPQVYTLPP SREEMTKNQV SLTCLVKGFY | 400 |
| PSDIAVEWES NGQPENNYKT TPPVLDSDGS FFLYSKLTVD KSRWQQGNVF | 450 |
| SCSVMHEALH NHYTQKSLSL SPG** | 475 |
| Wherein a small letter can be substituted with an amino | |
| acid selected from SEQ ID NOs: 9-11 in the corresponding | |
| position. | |
| SEQ ID NO: 25 (Tip Light Chain Consensus Sequence) | |
| METDTLLLWV LLLWVPGSTG DVVMTQSPIS LpVtLGqpAs IsCrSSQSLL | 50 |
| DSDGKTFLNW LQQrPGQsPr RLIYLVSKlD SGVPDRFSGS GSGTDFTLkI | 100 |
| SrveAEDVgV YYCWQGTHFP YTFGQGTKLE IKRTVAAPSV FIFPPSDEQL | 150 |
| KSGTASVVCL LNNFYPREAK VQWKVDNALQ SGNSQESVTE QDSKDSTYSL | 200 |
| SSTLTLSKAD YEKHKVYACE VTHQGLSSPV TKSFNRGEC** | 240 |

Wherein a small letter can be substituted with an amino
acid selected from SEQ ID NOs: 15-17 in the corresponding
position.
SEQ ID NO. 26: Non-limiting exemplary linker:
GPSL.
Non-limiting exemplary linker:
GPS.
SEQ ID NO. 28: Non-limiting exemplary linker:
PSLK.
SEQ ID NO. 29: Non-limiting exemplary linker:
GPSLK.
SEQ ID NO. 30: Non-limiting exemplary linker:
SLKL.
SEQ ID NO: 31 Mus musculus Wild-type HMGB1 protein
MGKGDPKKPRRKMSSYAFFVQTCREEHKKKHPDASVNFSEFSKKCSERWKTM
SAKEKGKFEDMAKADKARYEREMKTYIPPKGETKKKFKDPNAPKRPPSAFFLFCSE
YRPKIKGEHPGLSIGDVAKKLGEMWNNTAADDKQPYEKKAEKLKEKYEKDIAAYRAKGKP
DAAKKGVVKAEKSKKKKEEEEG EEDEEDEEEE EDEEDEDEEE DDDDE
SEQ ID NO: 32 Homo sapiens Wild-type HMGB1 protein
MGKGDPKKPRGKMSSYAFFVQTCREEHKKKHPDASVNFSEFSKKCSERWKTM
SAKEKGKFEDMAKADKARYEREMKTYIPPKGETKKKFKDPNAPKRPPS
AFFLFCSEYR
PKIKGEHPGLSIGDVAKKLGEMWNNTAADDKQPYEKKAAKLKEKYEKDIAAYRAKGKPD
AAKKGVVKAEKSKKKKEEEED EEDEEDEEEE EDEEDEDEEE DDDDE
(Human, reproduced from GenBank Accession No. CAE48262.1)
HMGB1 is a small protein of 215 amino acid protein (of
approx. 30 Kda) composed of 3 domains: two positively charged
domains the A and B box each one comprising of 80 amino acids
and a negatively charged carboxyl terminus the acidic C tail
which consists of approximately 30 consecutive aspartate and
glutamate residues. Bolded amino acids (amino acids 1-70)
depict the A Box domain. The italicized amino acids (about
amino acids 88-164) depict the B Box domain. The underlined
amino acids (amino acids 186-215) depict the C-tail domain.
SEQ ID NO: 33 Human IgD constant region, Uniprot: P01880
APTKAPDVFPIISGCRHPKDNSPVVLACLITGYHPTSVTVTWYMGTQSQPQRTFPEIQ
RRDSYYMTSSQLSTPLQQWRQGEYKCVVQHTASKSKKEIFRWPESPKAQASSVPTA
QPQAEGSLAKATTAPATTRNTGRGGEEKKKEKEKEEQEERETKTPECPSHTQPLGVY
LLTPAVQDLWLRDKATFTCFVVGSDLKDAHLTWEVAGKVPTGGVEEGLLERHSNG
SQSQHSRLTLPRSLWNAGTSVTCTLNHPSLPPQRLMALREPAAQAPVKLSLNLLASS
DPPEAASWLLCEVSGFSPPNILLMWLEDQREVNTSGFAPARPPPQPGSTTFWAWSVL
RVPAPPSPQPATYTCVVSHEDSRTLLNASRSLEVSYVTDHGPMK
SEQ ID NO: 34 Human IgG2 constant region, Uniprot: P01859
ASTKGPSVFPLAPCSRSTSESTAALGCLVKDYFPEPVTVSWNSGALTSGVHTFPAVLQ
SSGLYSLSSVVTVPSSNFGTQTYTCNVDHKPSNTKVDKTVERKCCVECPPCPAPPVA
GPSVFLFPPKPKDTLMISRTPEVTCVVVDVSHEDPEVQFNWYVDGVEVHNAKTKPR
EEQFNSTFRVVSVLTVVHQDWLNGKEYKCKVSNKGLPAPIEKTISKTKGQPREPQVY
TLPPSREEMTKNQVSLTCLVKGFYPSDISVEWESNGQPENNYKTTPPMLDSDGSFFL
YSKLTVDKSRWQQGNVFSCSVMHEALHNHYTQKSLSLSPGK
SEQ ID NO: 35 Human IgG3 constant region, Uniprot: P01860
ASTKGPSVFPLAPCSRSTSGGTAALGCLVKDYFPEPVTVSWNSGALTSGVHTFPAVL
QSSGLYSLSSVVTVPSSSLGTQTYTCNVNHKPSNTKVDKRVELKTPLGDTTHTCPRC
PEPKSCDTPPPCPRCPEPKSCDTPPPCPRCPEPKSCDTPPPCPRCPAPELLGGPSVFLFPP
KPKDTLMISRTPEVTCVVVDVSHEDPEVQFKWYVDGVEVHNAKTKPREEQYNSTFR
VVSVLTVLHQDWLNGKEYKCKVSNKALPAPIEKTISKTKGQPREPQVYTLPPSREEM
TKNQVSLTCLVKGFYPSDIAVEWESSGQPENNYNTTPPMLDSDGSFFLYSKLTVDKS
RWQQGNIFSCSVMHEALHNRFTQKSLSLSPGK
SEQ ID NO: 36 Human IgM constant region, Uniprot: P01871
GSASAPTLFPLVSCENSPSDTSSVAVGCLAQDFLPDSITLSWKYKNNSDISSTRGFPSV
LRGGKYAATSQVLLPSKDVMQGTDEHVVCKVQHPNGNKEKNVPLPVIAELPPKVSV
FVPPRDGFFGNPRKSKLICQATGFSPRQIQVSWLREGKQVGSGVTTDQVQAEAKESG
PTTYKVTSTLTIKESDWLGQSMFTCRVDHRGLTFQQNASSMCVPDQDTAIRVFAIPPS
FASIFLTKSTKLTCLVTDLTTYDSVTISWTRQNGEAVKTHTNISESHPNATFSAVGEAS
ICEDDWNSGERFTCTVTHTDLPSPLKQTISRPKGVALHRPDVYLLPPAREQLNLRESA
TITCLVTGFSPADVFVQWMQRGQPLSPEKYVTSAPMPEPQAPGRYFAHSILTVSEEE
WNTGETYTCVAHEALPNRVTERTVDKSTGKPTLYNVSLVMSDTAGTCY
SEQ ID NO: 37 Human IgG4 constant region, Uniprot: P01861
ASTKGPSVFPLAPCSRSTSESTAALGCLVKDYFPEPVTVSWNSGALTSGVHTFPAVLQ
SSGLYSLSSVVTVPSSSLGTKTYTCNVDHKPSNTKVDKRVESKYGPPCPSCPAPEFLG
GPSVFLFPPKPKDTLMISRTPEVTCVVVDVSQEDPEVQFNWYVDGVEVHNAKTKPR
EEQFNSTYRVVSVLTVLHQDWLNGKEYKCKVSNKGLPSSIEKTISKAKGQPREPQVY
TLPPSQEEMTKNQVSLTCLVKGFYPSDIAVEWESNGQPENNYKTTPPVLDSDGSFFL
YSRLTVDKSRWQEGNVFSCSVMHEALHNHYTQKSLSLSLGK
SEQ ID NO: 38 Human IgA1 constant region, Uniprot: P01876
ASPTSPKVFPLSLCSTQPDGNVVIACLVQGFFPQEPLSVTWSESGQGVTARNFPPSQD
ASGDLYTTSSQLTLPATQCLAGKSVTCHVKHYTNPSQDVTVPCPVPSTPPTPSPSTPP
TPSPSCCHPRLSLHRPALEDLLLGSEANLTCTLTGLRDASGVTFTWTPSSGKSAVQGP
PERDLCGCYSVSSVLPGCAEPWNHGKTFTCTAAYPESKTPLTATLSKSGNTFRPEVH
LLPPPSEELALNELVTLTCLARGFSPKDVLVRWLQGSQELPREKYLTWASRQEPSQG
TTTFAVTSILRVAAEDWKKGDTFSCMVGHEALPLAFTQKTIDRLAGKPTHVNVSVV
MAEVDGTCY
SEQ ID NO: 39 Human IgA2 constant region, Uniprot: P01877
ASPTSPKVFPLSLDSTPQDGNVVVACLVQGFFPQEPLSVTWSESGQNVTARNFPPSQD
ASGDLYTTSSQLTLPATQCPDGKSVTCHVKHYTNPSQDVTVPCPVPPPPPCCHPRLSL
HRPALEDLLLGSEANLTCTLTGLRDASGATFTWTPSSGKSAVQGPPERDLCGCYSVS
SVLPGCAQPWNHGETFTCTAAHPELKTPLTANITKSGNTFRPEVHLLPPPSEELALNE
LVTLTCLARGFSPKDVLVRWLQGSQELPREKYLTWASRQEPSQGTTTFAVTSILRVA
AEDWKKGDTFSCMVGHEALPLAFTQKTIDRMAGKPTHVNVSVVMAEVDGTCY
SEQ ID NO: 40 Human Ig kappa constant region, Uniprot: P01834
TVAAPSVFIFPPSDEQLKSGTASVVCLLNNFYPREAKVQWKVDNALQSGNSQESVTE
QDSKDSTYSLSSTLTLSKADYEKHKVYACEVTHQGLSSPVTKSFNRGEC
SEQ ID NO: 41: DNA, Artificial Sequence (M. tuberculosis
HupB primer 1)
GCGTGCATATGAACAAAGCAGAGCT CATTGACGT
SEQ ID NO: 42: DNA, Artificial Sequence (M. tuberculosis
HupB primer 2)
CGTGGCTCTTCCGCACGCTTTGCGACCCC GCCGAG
SEQ ID NO: 43 (tail-chimeric peptide IhfA3-IhfB2_{NTHI})
FLEEIRLSLESGQDVKLSGF-X-TLSAKEIENMVKDILEFISQ
SEQ ID NO: 44: Mus musculus Wild-type HMGB1 protein
MGKGDPKKPRRKMSSYAFFVQTCREEHKKKHPDASVNFSEFSKKCSERWKTM
SAKEKGKFEDMAKADKARYEREMKTYIPPKGETKKKFKDPNAPKRPPSAFFLFCSE
YRPKIKGEHPGLSIGDVAKKLGEMWNNTAADDKQPYEKKAEKLKEKYEKDIAAYRAKGKP
DAAKKGVVKAEKSKKKKEEEEG EEDEEDEEEE EDEEDEDEEE DDDDE
SEQ ID NO: 45 Homo sapiens Wild-type HMGB1 protein
MGKGDPKKPRGKMSSYAFFVQTCREEHKKKHPDASVNFSEFSKKCSERWKTM
SAKEKGKFEDMAKADKARYEREMKTYIPPKGETKKKFKDPNAPKRPPS
AFFLFCSEYR
PKIKGEHPGLSIGDVAKKLGEMWNNTAADDKQPYEKKAAKLKEKYEKDIAAYRAKGKPD
AAKKGVVKAEKSKKKKEEEED EEDEEDEEEE EDEEDEDEEE DDDDE
(Human, reproduced from GenBank Accession No. CAB48262.1)
HMGB1 is a small protein of 215 amino acid protein (of
approx. 30 Kda) composed of 3 domains: two positively
charged domains the A and B box each one comprising of 80
amino acids and a negatively charged carboxyl terminus the
acidic C tail which consists of approximately 30 consecutive
aspartate and glutamate residues. Bolded amino acids (amino
acids 1-70) depict the A Box domain. The italicized amino
acids (about amino acids 88-164) depict the B Box domain.
The underlined amino acids (amino acids 186-215) depict the
C-tail domain.
SEQ ID NO: 46, Artificial sequence, Linker sequence
PPKGETKKKF
SEQ ID NO: 47, human, mB Box-97 peptide
KDPNAPKRPPSAFFLFSSEYRPKIKGEHPGLSIGDVAKKLGEMWNNTAADDKQPYEK
KAEKLKEKYEKDIAAYRAKGKPDAAKKGVV
SEQ ID NO: 48, human, mB Box-97 peptide with C terminal
linker sequence
PPKGETKKKFKDPNAPKRPPSAFFLFSSEYRPKIKGEHPGLSIGDVAKKLGEMWNNT
AADDKQPYEKKAEKLKEKYEKDIAAYRAKGKPDAAKKGVV
SEQ ID NO: 49, human, IhfA, A tip fragment
NFELRDKSSRPGRNPKTGDVV
SEQ ID NO: 50, human IhfB, B tip fragment
SLHHRQPRLGRNPKTGDSVNL
SEQ ID NO: 51, E. Coli, H-NS protein
MSEALKILNNIRTLRAQARECTLETLEEMLEKLEVVVNERREEESAAAAEVEERTRK
LQQYREMLIADGIDPNELLNSLAAVKSGTKAKRAQRPAKYSYVDENGETKTWTGQ
GRTPAVIKKAMDEQGKSLDDFLIKQ
SEQ ID NO: 52, NTHI, H-NS protein
MNELVRGLTNLRSLRAAVRELTLEQAENALEKLQTAIEEKRANEAELIKAETERKER
LAKYKELMEKEGITPEELHKIFGTKTVSIQAKRAPRPAKYAFIDENGEHKTWTGQGR
TPRPIQNALNKGKSLSDFEI
SEQ ID NO: 53, Mycobacterium tuberculosis, H-NS
MPDPQDRPDSEPSDASTPPAKKLPAKKAAKKAPARKTPAKKAPAKKTPAKGAKSAP
PKPAEAPVSLQQRIETNGQLAAAAKDAAAQAKSTVEGANDALARNASVPAPSHSPV
PLIVAVTLSLLALLLIRQLRRR
SEQ ID NO: 54, Streptococcus pneumoniae H-NS protein
MNELVRSLTNLRSLRAAVRELTLEQAENALEKLQTAIEEKRANEAELIKAETERKER
LAKYKELMEKEGITPEELHEIFGTKTVSIRAKRPPRPAKYAFIDENGEHKTWTGQGRT
PRPIQNALNKGKSLSDFEI
SEQ ID NO: 55, Klebsiella pneumoniae H-NS protein
MSEALKILNNIRTLRAQARECTLETLEEMLEKLEVVVNERREEENAAAAEIEERTRKL
QQYREMLIADGIDPNELLSTMAAVKAGTKTKRAARPAKYSYVDENGETKTWTGQG
RTPAVIKKAMDEQGKSLDDFLI
SEQ ID NO: 56, Pseudomonas aeruginosa H-NS protein
MSLINEYRNTEQTIKELQARLASLQQDGRMKAELEFDTKLRALMSEYNKSLRDVIIL
LDPQAQNRSSKTPPTSGRRERQLKRYLNPNTSEVVETKGGNHKILKEWKTQFGADV
VESWLQS
SEQ ID NO: 57, (E. coli HupB, Genbank accession No.:
AP_001090.1, Last accessed Mar. 21, 2011)
MNKSQLIDKIAAGADISKAAAGRALDAIIASVTESLKEGDDVALVGFGTFAVKERAA
RTGRNPQTGKEITIAAAKVPSFRAGKALKDAVN

1. A method for preventing or treating an infection caused by a Nontuberculous mycobacterium (NTM) species in a subject comprising administering to the subject an effective amount of an antibody or an antigen-binding fragment thereof that binds to a tip region of a DNABII peptide.

2. A method for sensitizing a biofilm to an antibiotic agent or disrupting a biofilm, wherein the biofilm comprises a Nontuberculous mycobacterium (NTM) species, the method comprising contacting the biofilm with an antibody or an antigen-binding fragment thereof that binds to a tip region of a DNABII peptide.

3. The method of claim 1, wherein the antibody or the antigen-binding fragment thereof comprises one or more of:
a.
(i) a heavy chain (HC) immunoglobulin variable domain sequence comprising a sequence of amino acid (aa) 25 to aa 144 of SEQ ID NO: 21 or an equivalent thereof, and
(ii) a light chain (LC) immunoglobulin variable domain sequence comprising a sequence of aa 21 to aa 132 of SEQ ID NO: 22 or an equivalent thereof;
b.
(i) a heavy chain (HC) immunoglobulin variable domain sequence comprising a sequence of aa 25 to aa 144 of SEQ ID NO: 24 or an equivalent thereof, and
(ii) a light chain (LC) immunoglobulin variable domain sequence comprising a sequence of aa 21 to aa 132 of SEQ ID NO: 25 or an equivalent thereof;
c.
(i) a heavy chain complementarity-determining region 1 (CDRH1) comprising a sequence of GFTFRTY (SEQ ID NO: 58) (aa 50 to aa 56 of SEQ ID NO: 9 or 10 or 11 or 24),
(ii) a heavy chain complementarity-determining region 2 (CDRH2) comprising a sequence of GSDRRH (SEQ ID NO: 59) (aa 76 to aa 81 of SEQ ID NO: 9 or 10 or 11 or 24),
(iii) a heavy chain complementarity-determining region 3 (CDRH3) comprising a sequence of VGPYDGYYGEFDY (SEQ ID NO: 60) (aa 121 to aa 133 of SEQ ID NO: 9 or 10 or 11 or 24),
(iv) a light chain complementarity-determining region 1 (CDRL1) comprising a sequence of QSLLDSDGKTF (SEQ ID NO: 61) (aa 47 to aa 57 of SEQ ID NO: 15 or 16 or 17 or 25),
(v) a light chain complementarity-determining region 2 (CDRL2) comprising a sequence of LVS (aa 75 to aa 77 of SEQ ID NO: 15 or 16 or 17 or 25), and
(vi) a light chain complementarity-determining region 3 (CDRL3) comprising a sequence of WQGTHFP (SEQ ID NO: 62) (aa 114 to aa 120 of SEQ ID NO: 15 or 16 or 17 or 25);
d.
(i) a heavy chain complementarity-determining region 1 (CDRH1) comprising, or consisting essentially of, or yet further consisting of a sequence of GFTFSRYG (SEQ ID NO: 63) (aa 50 to aa 57 of SEQ ID NO: 12 or 13 or 14),
(ii) a heavy chain complementarity-determining region 2 (CDRH2) comprising, or consisting essentially of, or yet further consisting of a sequence of ISSGGSYT (SEQ ID NO: 64) (aa 75 to aa 82 of SEQ ID NO: 12 or 13 or 14),
(iii) a heavy chain complementarity-determining region 3 (CDRH3) comprising, or consisting essentially of, or yet further consisting of a sequence of ERHGGDGYWYFDV (SEQ ID NO: 65) (aa 121 to aa 133 of SEQ ID NO: 12 or 13 or 14),
(iv) a light chain complementarity-determining region 1 (CDRL1) comprising, or consisting essentially of, or yet further consisting of a sequence of QSLLDSDGKTF (SEQ ID NO: 61) (aa 47 to aa 57 of SEQ ID NO: 15 or 16 or 17 or 25),
(v) a light chain complementarity-determining region 2 (CDRL2) comprising, or consisting essentially of, or yet further consisting of a sequence of LVS (aa 75 to aa 77 of SEQ ID NO: 15 or 16 or 17 or 25), and
(vi) a light chain complementarity-determining region 3 (CDRL3) comprising, or consisting essentially of, or yet further consisting of a sequence of WQGTHFPYT (SEQ ID NO: 66) (aa 114 to aa 122 of SEQ ID NO: 15 or 16 or 17 or 25);
and/or
e.
a heavy chain (HC) immunoglobulin variable domain comprising the amino acid sequence of amino acids 25 to 144 of SEQ ID NO: 9; and
a light chain (LC) immunoglobulin variable domain comprising the amino acid sequence of amino acids 21 to 132 of SEQ ID NO: 15, SEQ ID NO: 16, or SEQ ID NO: 17.

4. The method of claim 1, wherein the antibody or the antigen-binding fragment thereof further comprises a constant region selected from the group of: an IgA constant region, an IgD constant region, an IgE constant region, an IgG constant region or an IgM constant region.

5. The method of claim 4, wherein the constant region is an IgG1 constant region.

6. The method of claim 1, wherein the antigen-binding fragment is selected from the group of Fab, F(ab′)₂, Fab′, scFv, or Fv.

7. The method of claim 3, wherein the equivalent to an amino acid sequence comprises a polypeptide having at least 80% amino acid identity to the amino acid, or wherein an equivalent to the amino acid sequence comprises a polypeptide that is encoded by a polynucleotide that hybridizes under conditions of high stringency to the complement of the polynucleotide encoding the amino acid sequence.

8. The method of claim 1, wherein the antibody or the antigen-binding fragment thereof further comprises a modification, optionally selected from the group of PEGylation, a PEG mimetic, polysialyation, HESylation or glycosylation.

9. (canceled)

10. The method of claim 2, wherein the antibiotic agent comprises amikacin, azithromycin, dactinomycin, bleomycin, daunorubicin, doxorubicin, idarubicin, mithramycin, mitomycin, mitoxantrone, plicamycin, methicillin, vancomycin, daptomycin, mupirocin, penicillin, cloxacillin, erythromycin, clarithromycin, roxithromycin, telithromycin, spiramycin, fidaxomicin, rifampin, ethambutol, streptomycin or anthramycin.

11. The method of claim 2, further comprising contacting the biofilm with an effective amount of a synthetic or recombinant polypeptide comprising, or consisting essentially of, or yet further consisting of mB Box-97 that consists of the amino acids 90 to 176 or the amino acids 80-176 from the coding sequence of the native human HMGB1 protein shown as SEQ ID NO: 45 with a cysteine to serine point mutation at amino acid 106 or an equivalent thereof.

12. (canceled)

13. The method of claim 11, wherein the synthetic or recombinant polypeptide consists of SEQ ID NO: 48.

14. The method of claim 2, further comprising contacting the biofilm with an effective amount of a DNA-binding agent, optionally selected from a Histone-like Nucleoid Structuring protein (H-NS), a polyamine, or a polycation.

15. (canceled)

16. The method of claim 14, wherein the H-NS comprises an amino acid sequence having at least 60% (e.g., at least 60, 65, 70, 75, 80, 85, 90, 95, 99% or more) identity to an amino acid sequence selected from SEQ ID NOs: 51-56.

17. The method of claim 1, further comprising administering to the subject an effective amount of an at least one antibiotic agent selected from amikacin, azithromycin, dactinomycin, bleomycin, daunorubicin, doxorubicin, idarubicin, mithramycin, mitomycin, mitoxantrone, plicamycin, methicillin, vancomycin, daptomycin, mupirocin, penicillin, cloxacillin, erythromycin, clarithromycin, roxithromycin, telithromycin, spiramycin, fidaxomicin or anthramycin.

18. The method of claim 1, further comprising administering to the subject an effective amount of a synthetic or recombinant polypeptide comprising, or consisting essentially of, or yet further consisting of mB Box-97 that consists of the amino acids 90 to 176 or the amino acids 80-176 from the coding sequence of the native human HMGB1 protein shown as SEQ ID NO: 45 with a cysteine to serine point mutation at amino acid 106 or an equivalent thereof.

19. (canceled)

20. The method of claim 18, wherein the synthetic or recombinant polypeptide consists of SEQ ID NO: 48.

21. The method of claim 1, further comprising contacting the biofilm with an effective amount of a DNA-binding agent selected from a Histone-like Nucleoid Structuring protein (H-NS), a polyamine, or a polycation.

22. (canceled)

23. The method of claim 21, wherein the H-NS comprises an amino acid sequence having at least 60% (e.g., at least 60, 65, 70, 75, 80, 85, 90, 95, 99% or more) identity to an amino acid sequence selected from SEQ ID NOs: 51-56.

24. The method of claim 1, wherein the NTM species is M. abscessus (Mab), M. avium (Mav), M. intracellulare, or M. chimaera.

25. (canceled)

26. The method of claim 1, wherein the subject is suffering from chronic obstructive pulmonary disease (COPD), primary ciliary dyskinesia, or cystic fibrosis (CF).

27. (canceled)
